(19) United States
(12) Patent Application Publication
(10) Pub. No.: US 2011/0212475 A1
(43) Pub. Date: Sep. 1, 2011

(54) Fluorescently Or Spin-Labeled Kinases For Rapid Screening And Identification Of Novel Kinase Inhibitor Scaffolds

(76) Inventors: Daniel Rauh, Worms (DE); Jeffrey Raymond Simard, Salem, MA (US); Matthäus Getlik, Paderborn (DE)

(21) Appl. No.: 13/011,099

(22) Filed: Jan. 21, 2011

Related U.S. Application Data
(63) Continuation-in-part of application No. PCT/EP2009/005364, filed on Jul. 23, 2009.
(60) Provisional application No. 61/083,335, filed on Jul. 24, 2008.

(30) Foreign Application Priority Data
Jul. 24, 2008 (EP) 08013340.8
Nov. 21, 2008 (EP) 08020341.7
Apr. 17, 2009 (EP) 09005493.3

(51) Int. Cl. C12Q 1/48; C12N 9/96

(52) U.S. Cl. 435/15; 435/188

(57) ABSTRACT

The present invention relates to a kinase labeled at an amino acid having a free thiol or amino group, wherein said amino acid is naturally present or introduced in the activation loop of said kinase, with (a) a thiol- or amino-reactive fluorophore sensitive to polarity changes in its environment; or (b) a thiol-reactive spin label, an isotope or an isotope-enriched thiol- or amino-reactive label, such that said fluorophore, spin label, isotope or isotope-enriched label does not inhibit the catalytic activity and does not interfere with the stability of the kinase. The invention furthermore relates to a method of screening for kinase inhibitor, a method of determining the kinetics of ligand binding and/or of dissociation of a kinase inhibitor and a method of generating mutated kinases suitable for the screening of kinase inhibitors using the kinase of the present invention.

### RELATED APPLICATIONS AND INCORPORATION BY REFERENCE

This application is a continuation-in-part application of international patent application Serial No. PCT/EP2009/005364 filed 23 Jul. 2009, which published as PCT Publication No. WO 2010/009886 on 28 Jan. 2010, which claims benefit of European patent application Serial Nos. 08013340.8, 08020341.7 and 09005493.3, filed 24 Jul. 2008, 21 Nov. 2008 and 17 Apr. 2009 and U.S. provisional patent application Ser. No. 61/083,335 filed 24 Jul. 2008.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to a kinase labeled at an amino acid having a free thiol or amino group, wherein said amino acid is naturally present or introduced in the activation loop of said kinase, with (a) a thiol- or amino-reactive fluorophore sensitive to polarity changes in its environment; or (b) a thiol-reactive spin label, an isotope or an isotope-enriched thiol- or amino-reactive label, such that said fluorophore, spin label, isotope or isotope-enriched label does not inhibit the catalytic activity and does not interfere with the stability of the kinase. The invention furthermore relates to a method of screening for kinase inhibitors, a method of determining the kinetics of ligand binding and/or dissociation of a kinase inhibitor and a method of generating mutated kinases suitable for screening of kinase inhibitors using the labeled kinase of the present invention.

### BACKGROUND OF THE INVENTION

Protein kinases play a critical role in regulating many aspects of cellular function. For this reason, they are widely considered to be among the most attractive targets for therapeutic drug development. To date, strategies for inhibiting kinases have been based primarily on compounds designed to bind directly at the natural substrate (i.e. ATP) binding site. These are known as ATP-competitive inhibitors, also termed Type I inhibitors. Recently, inhibitors which bind exclusively to sites adjacent to the ATP-binding pocket and thereby induce an inactivating conformational change in the protein have been found. These compounds are known as allosteric, or Type III, inhibitors. Allosteric inhibitors which bind to this allosteric site but also extending into the ATP-binding pocket of a kinase are also known and termed Type II inhibitors. Examples for the latter are imatinib (Gleevec), sorafenib (Nexavar) and BIRB-796.

Aberrantly regulated kinases play causative roles in many diseases, and the most common strategy for regulating unwanted kinase activity is the use of ATP competitive (Type I) inhibitors. However, the development of drugs which bind to a single specific kinase has been hampered by the high sequence and structural homology in the ATP binding pocket of all kinases, resulting in the low specificity of such inhibitors. A less-conserved hydrophobic pocket adjacent to the ATP binding site was first identified in p38α MAP kinase (Pargellis et al., 2002) and MEK kinases (Ohren et al., 2004) and found to be an allosteric binding site. Inhibition at this site has since been found in several other kinases including Aurora, EGFR, Src, Abl. Kinases are typically in the active conformation (DFG-in) with the activation loop open and extended, allowing ATP and other molecules to bind. Alternatively, the adjacent allosteric site is only available in the inactive conformation (DFG-out) in which the activation loop shifts conformation and interferes with both ATP binding to the ATP-pocket and substrate binding to the allosteric binding site. Various tight binding inhibitors have recently been developed for p38α which either bind in the allosteric pocket exclusively (Type III) or extend from this pocket into the ATP binding site (Type II). The availability of structural information for the inactive state of these kinases has intensified the search for new drug scaffolds which bind to this site with high affinity and increased specificity. Methods for discriminating between compounds which bind in each site are currently limited (Annis et al., 2004; Vogtherr et al., 2006). Furthermore, rapid and feasible high-throughput screening methods for the identification of Type I, II and III inhibitors are not yet available.

The attachment of fluorophores to proteins is a well-established approach used to detect conformational changes in protein structure in response to ligand binding. In addition to the commercially-available probe acrylodan-labeled fatty acid binding protein (ADIFAB; Molecular Probes), which measures the concentration of unbound fatty acids in buffer (Richieri et al., 1999), this approach has been applied to various other proteins including acetylcholine binding protein (Hibbs et al., 2004), interleukin-1β (Yem et al., 1992) and various sugar and amino acid binding proteins (de Lorimier et al., 2002).

Citation or identification of any document in this application is not an admission that such document is available as prior art to the present invention.

### SUMMARY OF THE INVENTION

It would be desirable to have versatile means and methods for screening for specific kinase inhibitors. The solution to this technical problem is achieved by providing the embodiments characterized in the claims.

Accordingly, the present invention relates to a kinase labeled at an amino acid having a free thiol or amino group, wherein said amino acid is naturally present or introduced in the activation loop of said kinase, with (a) a thiol- or amino-reactive fluorophore sensitive to polarity changes in its environment; or (b) a thiol-reactive spin label, an isotope or an isotope-enriched thiol- or amino-reactive label, such that said fluorophore, spin label, isotope or isotope-enriched label does not inhibit the catalytic activity and does not interfere with the stability of the kinase.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1

p38α has been crystallized in its active (DFG-in) and inactive state (DFG-out) (A). The pyrazolo-urea compound BIRB-796 is a Type II inhibitor which extends between the ATP and allosteric binding sites of p38α. Attachment of acrylodan (modeled here as a tryptophan) to a selected Cys (B) mutation in the activation loop should detect conformational changes that result from the binding of Type II and III inhibitors (C). Upon binding, BIRB-796 alters the conformation of the activation loop (red) (D).

FIG. 2

The binding of allosteric inhibitors results in a large decrease in acrylodan emission at 468 nm and a characteristic red-shift to 514 nm in the ligand-bound (inactive) state.

FIG. 3

Real-time and endpoint fluorescence measurements using ac-p38α labeled on the activation loop. Acrylodan emission at 468 nm decreases in a dose-dependent manner upon binding of BIRB-796 (A and C). Fluorescence traces follow first-order decay kinetics and can be plotted to determine kₒₙ and k_{off} for BIRB-796 (B and D). Endpoint equilibrium measurements can also be made to obtain the Kd of binding. Raw fluorescence data (R=514 nm/468 nm) were plotted to show the saturation of the inactive state (E and G). The Kd was determined by using a logarithmic scale plotted against R and fractional occupancy (F and H).

FIG. 4

Titration of sorafenib (inhibitor for b-Raf and p38α), lapatinib (inhibitor for EGFR and HER2) (inhibitor for Abl, c-Kit) and imatinib with ac-p38α. Fluorescent-tagged p38α was incubated with various inhibitor concentrations overnight prior to making endpoint measurements (left). Imatinib and lapatinib did not bind to ac-p38α (as expected) in the concentration range examined, while sorafenib bound tightly with a K_{d} ˜56 nM. The structures of each inhibitor are also shown (right).

FIG. 5

Dissociation of BIRB-796 and 1, RL8 (MG001) from ac-p38α and direct measurements of k_{off}. BIRB-796 or 1, RL8 were mixed with ac-p38α (0.1 μM) in a 1:1 ratio (for a cross-reference index of all compounds used in the present invention see table 7 below). After sufficient incubation time, 1 μM unlabeled p38α was added to a rapidly stirring cuvette to induce the dissociation of inhibitor. Acrylodan fluorescence was monitored at 468 nm. Under these conditions, the k_{off} of BIRB-796 (left) and 1, RL8 (right) were measured to be 4.54×10⁻⁵ s⁻¹ and 1.08×10⁻² s⁻¹, respectively.

FIG. 6

Binding of BIRB-796 and 1, RL8 to ac-p38α and direct measurement of kₒₙ. BIRB-796 or 1, RL8 was mixed with ac-p38α (0.1 μM) in various ratios (1-4:1 inhibitor:protein). Acrylodan fluorescence was monitored at 468 nm following the addition of inhibitor to a rapidly stirred cuvette. Under these conditions, the k_{obs} of BIRB-796 (left) and 1, RL8 (right) were measured at each dose and used to determine kₒₙ values of 4.46×10³ M⁻¹s⁻¹ and 9.27×10³ M⁻¹ s⁻¹, respectively.

FIG. 7

Binding of BIRB-796, staurosporine and SB203580 to ac-p38α. The high affinity ATP-competitive inhibitor of p38α, SB203580, binds with a K_{d} ˜15 nM while staurosporine is not detected (left). Each inhibitor was incubated with 50 nM ac-p38α overnight to obtain the data for binding curves. For real-time kinetic measurements, a single dose of each inhibitor (10 μM) was added to ac-p38α (0.1 μM). ATP-competitive inhibitors produce an instantaneous change in fluorescence (right). Weaker binding ATP-competitive inhibitors (K_{d}>20 nM) induce smaller changes (smaller magnitude) or no change at all (not shown).

FIG. 8

Binding of BIRB-796 to ac-p38α in different HTS formats. BIRB-796 was incubated with acp38α overnight at 4° C. In 96-well plates, 1 nM-2 μM inhibitor was used while 10 nM-20 μM inhibitor was used in 384-well plates. Under these conditions, the Kd of BIRB-796 was ˜27 nM in a 96-well format (left) and ˜76 nM in a 384-well format (right).

FIG. 9

Binding experiment of BIRB-796 and ac-p38α to determine time-dependent inhibition. The protein ligand mixture was incubated for 24 hours at 4° C. with fluorescence measurements taken at various time intervals Plotted binding curves reveal the expected time-dependence of BIRB-796 inhibition.

FIG. 10

Core structure of compounds in the used DFG-out library. The proposed binding mode of these compounds is also shown (left) and orientated with BIRB-796 for comparison (right). Regions extending into the ATP site and allosteric site are variable.

FIG. 11

Characterization of a DFG-out compound library hit. The structure of 85-C8 is shown (a) with the conserved moiety shared by all hits highlighted (red=. Using the cuvette method, 100 nM ac-p38alpha was incubated overnight with 10-100 μM of 85-C8 and emission spectra were collected (B) and binding curves were generated (C). Real-time fluorescence measurements were also performed by monitoring emission of acrylodan at 468 nm and adding a single dose of 85-C8 (5-30 μM) (D). Addition of these compounds resulted in a mixed fluorescence response with an initial rapid fluorescence change followed by slow first-order decay. Both the fast (E) and slow (F) phases are dose-dependent.

FIG. 12

Using the ac-p38α assay to predict the binding mode of a DFG-out compound library hit. The binding mode of structurally similar compounds (A), dasatinib and INH-29, is predominantly ATP-competitive as a result of H-bonding to the hinge region of the kinase. Alignment of 87-F9, 2 (orange) with dasatinib (magenta) reveals strong conservation of several H-bond donors or acceptors in the drug scaffold which interact with the hinge region of the kinase (B). The long extension of the library hit structure may allow it to enter the allosteric pocket (C). Dasatinib lacks this feature and only produces an instantaneous fluorescence change when added to ac-p38α, indicative of totally ATP-competitive binding. The binding modes of dasatinib INH-29 are adapted from Andersen et al. (6). (D) Affinity of compound library hits to p38α, (E) Crystal structure of hit 87H9 bound to p38α, (F) Screening scheme applied for a compound library of 35,000 compounds, (G) Monitoring of ratiometric values of primary HTS screen, (H) IC50 determinations and SAR studies on HTS 14 and HTS 15 as well as derivatives HTS 14a-e and HTS 15a-c, (I) Kinetic measurements of HTS 12 and HTS 13, (J) Binding mode of ligand HTS 12 to acrylodan-labelled p38α.

FIG. 13
A. Amino acid sequence alignment of the activation loops of several kinases. Residues are coloured according to their similar properties; hydrophobic/non-charged (red), polar/acidic (blue), polar/basic (pink), polar/uncharged (green). The length of the loop appears at the end of each sequence. Specific motifs are boxed in, labeled and described in the text. The labeling position is marked (*) according to the position chosen for p38α. The first half of the activation loop closest to the DFG motif is the shortest in length in p38α. Structural information for other kinases which have longer activation loops reveals that the residue directly following the DFG motif (#) aligns well with the site chosen for ac-p38α.
B. Sequence alignment of several kinase activation loops guides labeling of the DFG+1 and DFG+2 positions (bold text) Alignments were performed using Clustal W (Larkin et al., 2007). Regions that are highly conserved or crucial to kinase structural stability or enzymatic activity are shown (boxed regions). The observed differences in this alignment when compared to panel A are due to the use of different kinases in this alignment. Boxed regions in both panels were arbitrarily placed around the corresponding functional regions of the activation loop as described.

FIG. 14

Structural alignment of kinases with p38α for determining the fluorophore attachment site. cAbl kinase was aligned with active (A) and inactive (B) p38α to reveal the conformations adopted by the lengthy activation loop and suggests that the first position after the DFG motif is best for labeling in such kinases. This residue is positioned most similarly to the labeled site in p38α, which is one residue farther away from the DFG motif. In EGFR, the positioning in the DFG-out conformation was used to select a unique position which is much farther from the DFG motif (C). After formation of the unique inactive state of EGFR, this residue is positioned most similar to the labeled site of inactive p38α.

FIG. 15

Co-crystal structure of ac-p38α in complex with Type II inhibitor sorafenib. Electron density maps of sorafenib (pink) and acrylodan (white) are contoured at 1σ. Possible hydrogen bonding interactions are highlighted by dotted lines (a). Structural alignment of the ac-p38α-sorafenib complex with the b-Raf-sorafenib complex.

FIG. 16

a to c: HPLC and mass spectrometric analysis of the acrylodan-labeled chicken cSrc kinase domain. The predicted fragment mass of the desired labelled peptide (839 Da) was determined using the software program Peptide Cutter (www.expasy.org/tools/peptidecutter). The amino acid sequence of the desired fragment is N′-VADFGCAR-C′ and has an expected mass of 839 Da (or 1064 Da when acrylodan is conjugated to the Cys). (a) Singly charged ion ([M+H]⁺) mass chromatogram of m/z 839.0, the unlabeled peptide fragment. A high intensity peak appears after 25.51 min containing this expected fragment and this peak was only observed for the unlabeled cSrc kinase domain. (b) Singly charged ion ([M+H]⁺) mass chromatogram of m/z 1064.0, the labeled peptide fragment. A high intensity peak appears after 40.90 min containing this expected labelled fragment and this peak was only observed for the labelled cSrc kinase domain. Together, both results suggest 100% labelling of the desired Cys residue since the expected masses were only observed in the expected kinases (labelled or unlabeled). (c) MS/MS spectrum of the doubly charged ion of the labelled cSrc fragment (m/z=532.9) with labelled b and y series (Roepsdorff nomenclature). Peaks for the complete y series are labelled, confirming the complete peptide sequence. In the b series, only b6 is missing (b1 is too small to be detected).

d to f: HPLC and mass spectrometric analysis of acrylodan-labeled and unlabeled human p38α. The predicted fragment mass of the desired labeled peptide (1161 Da) was determined using the software program Peptide Cutter (www.expasy.org/tools/peptidecutter). The amino acid sequence of the desired fragment is N′-ILDFGLCR-C′ and has an expected mass of 936 Da (or 11161 Da when acrylodan is conjugated to the Cys). ESI-MS of the labeled p38α (41422 Da) reveals a mass shift of 225 Da relative to the unlabeled kinase upon labeling of the protein with a single acrylodan molecule (d). Unlabeled kinase is still present in low abundance (41197 Da). Singly charged ion ([M+H]⁺) mass chromatogram of m/z 1062.0, the labeled peptide fragment (e). A high intensity peak appears after 47.36 min containing this expected labeled fragment and this peak was only observed for labeled p38α kinase. Together, results in Panels d & e demonstrate nearly complete 1:1 labeling of the desired Cys residue. MS/MS spectrum of the doubly charged ion of the labeled p38α fragment (m/z=581.9) with labeled b and y series (Roepsdorff nomenclature) (f). Peaks for the complete b series are labeled (b₁ and b₈ are too small and large, respectively, to be detected), confirming the complete peptide sequence. In the y series, only y₁ is missing (y₈ is too large to be detected). See Methods for further details.

FIG. 17

Fluorescence characterization of acrylodan-labeled chicken cSrc. (a) A 3 mL suspension of 100 nM labeled cSrc was placed into a standard fluorescence spectrometer and excited at 386 nm to record the emission spectrum in the absence of ligand (solid line), 11c, RL46 (shown as 3c in the figure) (solid squares) or dasatinib (open squares). Intensities at λ475 nm and λ505 nm were chosen for quantitating changes in fluorescence intensity associated with inhibitor binding with λ445 nm serving as an internal fluorescence reference allowing for ratiometric fluorescence readouts. Inhibitors of the Type I scaffold (dasatinib) give a different fluorescence response than those of Type II scaffolds 11c. Type III binders produced similar spectra to Type II binders (not shown). (b) Binding curves for dasatinib determined using the fluorescence emission ratios (R) of λ445/λ475 nm (left) and λ475/λ505 nm (right). (c) Binding curves for 11c determined using the fluorescence emission ratios (R) of λ445/λ475 nm (left) and λ475/λ505 nm (right). Since λ475 and λ505 respond similarly to DFG-out binders, the result is flat line.

FIG. 18

Initial Type III screening hits and binding mode prediction. (a) Compound screens were performed at 10 and 50 μM concentrations as described in the Methods section. Acrylodan was excited at 386 nm. Following measurements of 384-well plates at λ445, λ475 and λ505, ratiometric values (λ445/λ475) were calculated and compared to the responses obtained from saturating concentrations of a known DFG-out binder of cSrc, imatinib. R values are shown for (3-7) (listed as 1a-e, respectively, in the figure). Only (3, RL57) and (6, RL35) were detected at 10 μM while (3-6) were detected at 50 μM with the following decreasing affinity ranking: (3, RL57/6, RL35)>(5, RL38)>(4, RL37). None of these compounds reach 50% of the maximal fluorescence change of imatinib at 50 μM. The fluorescence ratio did not respond to (7, RL19) at any concentration, since it was designed to be too bulky to bind to the allosteric pocket of cSrc (and also p38α—see FIG. 19). (b) Inhibitor types can be discriminated by acrylodan-labeled cSrc using R=λ475/λ505. The mean value of R=λ475/λ505 for labeled cSrc in the absence of ligand (apo cSrc) is 1.013 (line). The same values were calculated for all Type I, II and III inhibitors studied at the maximum elicited fluorescence response and are plotted as mean values±standard deviation for each inhibitor type.

FIG. 19

Screening of a focused library of pyrazoloureas reveals binding to cSrc, drug resistant cSrc-T338M and p38α. (a) Structures of pyrazoloureas (3-7) (listed as 1a-e, respectively, in the figure) and 4-aminoquinazolines (8, RL55; 9, RL56; 10, RL6) (listed as 2a-c, respectively, in the figure) are shown. (b) IC₅₀ values for inhibited enzyme activity (in μM) for a panel of inhibitors against wild type (cSrc) and drug resistant cSrc (cSrc-T338M). K_{d} values (in μM) for the same panel of pyrazoloureas in cSrc and p38α measured with the described fluorescence-based binding assay. Pyrazoloureas (3-6) are potent inhibitors of p38α. Inhibitor (3, RL57) demonstrates balanced inhibition of wild type and drug resistant cSrc-T338M. Bulky naphthyl derivative (5, RL38) weakly inhibits cSrc but fails to inhibit cSrc-T338M most likely due to a steric clash with the larger gatekeeper residue. The sterically demanding (7, RL19) does not fit into the allosteric site of cSrc or p38α and serves as a negative binding control. Quinazolines (8-10) are weak Type I inhibitors of cSrc that bind to the hinge region of the kinase and were also detected with the fluorescent assay in cSrc (but very weakly sensed in fluorescent p38α). The large bromo-phenyl moiety clashes with the gatekeeper side chain in drug resistant cSrc-T338M and results in significant drop in affinity (Michalczyk et al., 2008). A similar clash also occurs with dasatinib, but not with staurosporine which binds away from the gatekeeper. [Note: ‘*’ denotes compounds for which K_{d} values were not measurable (nm) due to high interference by intrinsic compound fluorescence. ‘**’ denotes Type I compounds that either do not bind at 10 μM (nb) or are weakly sensed (10-fold higher K_{d} than previously reported) by acrylodan-labeled p38α. Fluorescent p38α exhibits an insensitivity to Type I binders, unlike fluorescent cSrc, while both fluorescent kinases serve as excellent sensors for DFG-out binders.]

FIG. 20

Structure-based design of potent Type II hybrid inhibitors of cSrc kinase. (a) cSrc in complex with the Type III inhibitor (4, RL37). Electron density maps (2 Fo−Fc) of cSrc (grey) and (4, RL37) (red) are contoured at 1σ. Hydrogen bonding interactions of the inhibitor with the DFG motif (orange) and helix C (blue) are highlighted by dotted lines. The hinge region (pink) of the kinase domain (represented by M341) is not contacted by the inhibitor. (b) The co-crystal structures of cSrc in complex with the Type III inhibitor (3, RL57) (grey) aligned with the structure of cSrc in complex with an ATP-competitive 4-aminoquinazoline (green) (PDB entry 2QLQ16) provided the rationale for structure-based drug design. The quinazoline core binds to the hinge region of the kinase while the pyrazolourea exclusively binds to the allosteric site of the kinase. The plane of the phenyl moieties of both inhibitors align adjacent to the gatekeeper residue, Thr338 in chicken cSrc. (c) Rationally designed Type II inhibitors based on the binding modes of Type 14-aminoquinazolines and Type III pyrazoloureas bound to cSrc. According to the notion of fragment-based drug discovery (Shuker et al., 1996; Nienaber et al., 2000), combining the two weak binders chemically should result in significantly higher binding affinities.

FIG. 21

Focused library of rationally designed Type II inhibitors. (a) Structures of 1,4-linked 11a-c (shown as 3a-c in the figure) and 1,3-linked 11d-e (shown as 3d-e in the figure) quinazoline-pyrazolourea hybrid compounds are shown. (b) IC₅₀ and K_{d} values (in μM) for a panel of inhibitors against cSrc wild type, drug resistant cSrc-T338M and p38α. 1,4-substituted hybrids show best balance of potency and selectivity for cSrc wild type and cSrc-T338M. The R1 substituents in position 6 of the quinazoline core are important determinants for potency in cSrc and render a clear SAR with 11c, RL46 being the most potent hybrid compound for both cSrc wild type and drug resistant cSrc-T338M. 1,3-fusion of the inhibitor cores of 11d-e directs selectivity towards p38α and significantly decreases affinity to drug resistant cSrc-T338M.

FIG. 22

Chemical synthesis of 1,3- and 1,4-substituted hybrid compounds. i) Formamidine acetate, 2-methoxyethanol, 132° C.; ii) 4-hydroxy-6-nitroquinazoline (9), SOCl2, cat. DMF, reflux; iii) pyrazolourea-phenylenediamine (5), DIPEA, DCM, rt; iv) Ammonium formate, Pd/C, EtOH, reflux; v) propionyl chloride, DIPEA, THF, 0° C. Note: the compounds shown as 3a-c in the figure are compounds 11a-c as used throughout this application, while compounds shown as 3d-e are compounds 11d-e as used throughout this application.

FIG. 23

1,4-substituted hybrid compound 11b, RL45 (shown as 3b in the figure) in complex with wild type chicken cSrc and drug resistant cSrc-T338M shows different binding modes. Stereodiagrams of the experimental electron densities (ligand red, protein grey) of cSrc-RL45 (a) and cSrc-T338M-RL45 (b) at 2.6 Å resolutions are shown (2 Fo−Fc map contoured at 1σ). Hydrogen bonding interactions of the inhibitors with helix C (blue), the DFG-motif (orange) and the hinge region (pink) are shown by red dotted lines. The kinase domain is in the inactive conformation and the pyrazolourea moiety resides in the allosteric site flanked by helix C and the DFG-motif. N1 of the quinazoline makes a direct hydrogen bond to the main chain amide of M341, which is a common interaction formed between anilino-quinazolines and the hinge region of several other protein kinase domains. In both complexes, the central phenyl moiety which links the quinazoline scaffold with the pyrazolourea fragment interacts with the side chain of F405 (DFG motif) in a favorable edge-to-face orientation. (c) van der Waals radii of the inhibitor (mesh), the gatekeeper residues T338/M338 (pink spheres) and the side chain of F403 (orange spheres) explain conformational changes of the central phenyl moiety of the inhibitor to bypass steric clashes with the side chain of M338, allowing (11b, RL45) to bind to drug resistant cSrc-T338M. (d) A larger side chain at the gatekeeper position results in a 90° flip of the central phenyl moiety of the inhibitor. Likewise, the side chain of F405 is rotated by 90° to keep the electrostatically favorable edge-to-face orientation of both π-electron systems conserved (Hunter et al., 1991).

FIG. 24

1,3-substituted hybrid docked to drug resistant cSrc-T338M. Compound 11e, RL62 (shown as 3e in the figure) was docked manually into the structure of wild type cSrc-RL45 (a) and drug resistant cSrc-T338M-RL45 (b) complexes. Care was taken to conserve the essential hydrogen bonding interaction of the quinazoline N1 with the backbone of the hinge region and occupation of the allosteric site by the pyrazolourea moiety. The inhibitor adopts a binding mode well tolerated by a small gatekeeper residue (T338). (c) In drug resistant cSrc-T338M the central 1,4-substituted phenyl element of 11a-c (shown as 3a-c in the figure) can freely rotate to adopt to a larger gatekeeper residue. (d) Free rotation of this crucial element in 1,3-substituted hybrid compounds is not favoured and would result either in loss of the backbone hydrogen bond or displacement of the pyrazolourea from the allosteric site. Decreased inhibitor flexibility helps to explain why binding of 11d, RL61 (shown as 3d in the figure) and 11e to drug resistant cSrc-T338M is significantly compromised.

FIG. 25

Reduction of cell-to-cell-contacts and cell proliferation in PC3 and DU145 cells by (11c, RL46). (a) PC3 and DU145 cells were treated for five hours with (11c, RL46) (1, 2, 5 and 10 μM), dasatinib (100 nM), or vehicle (DMSO). Cells were lysed and blotted for indicated proteins. pSrc and pFAK levels are markedly reduced in response to treatment with (11c, RL46) and dasatanib (left panel). Total expression of FAK was unchanged while cSrc expression was increased in both cell lines. (b). Cell-to-cell contacts visualized by light microscopy at 10× magnification. PC3 and DU145 cells show markedly reduced cell-to-cell-contacts and fewer intact cells after 24 hours treatment with (11c, RL46) (10 μM) or dasatinib (100 nM).

FIG. 26

Heat map of kinase selectivity for hybrid compound (11b, RL45). (11b, RL45) (5 μM) was screened against a panel of 64 kinases (Ambit Biosciences) and their binding strengths scored accordingly (color scale shown above; strong binding green, no binding red). The results show a clear preference of tyrosine kinases (TK) for (11b, RL45). TKs with large hydrophobic gatekeepers can accommodate the inhibitor. Most serine-threonine kinases (STK) in the screen did not bind (11b, RL45), possibly due to incompatible binding site geometries for the 1,4-substituted hybrid. Sequence alignment analysis of the gatekeeper residues reveals that several of these non-CMGC STKs also contain large hydrophobic gatekeepers, which are less prominent in CMGC family STKs, which showed selectivity for (11b, RL45). The software GenePattern (Reich et al., 2006) was used to cluster kinases according to their inhibition profile. Data was not normalized; the clustering is hierarchical and based on Euclidian distances. Kinase domain sequences were aligned with ClustalW (Larkin et al., 2007) to produce the cladogram on the top part of the figure (drawn with FigTree, by Andrew Rambaut). Each gatekeeper is color-coded to the average of the Ambit scores obtained for 11b, RL45 by kinases possessing this gatekeeper. Serine/threonine kinase types are highlighted in gray.

FIG. 27

The binding and dissociation kinetics of (11a, RL44) in acrylodan-labeled cSrc. Acrylodan-labeled cSrc (50 nM) was placed into cuvettes with a rapidly stirring mini stir bar while monitoring acrylodan fluorescence (left panel). A single dose of (11a, RL44) (300 nM) was added to the sample using the injection port above the sample and triggered a fluorescence decrease which was well fitted to a first order decay function (k_{obs} of binding=0.087 s⁻¹; t_{1/2}=7.95 sec). The sample was allowed to reach equilibrium before adding a 10-fold excess of unlabeled cSrc to extract the bound (11a, RL44) from the labeled cSrc. Addition of excess unlabeled kinase resulted in fluorescence increase to its initial level which was also well fitted with a first order decay function (k_{obs} of dissociation=0.045 s⁻¹; t_{1/2}=15.32 sec). A single dose of (11a, RL44) (300 nM) added to acrylodan-labeled p38α under similar conditions results in slower binding of the ligand by comparison (right panel). The results for cSrc support published observations in which DFG-out binders have slower off rates than on rates, thereby contributing to their higher affinities to the kinase. The slower on rates of (11a, RL44) in p38α might be attributed to the more flexible helix C in tyrosine kinases such as cSrc, thus making the allosteric pocket more accessible to the ligand. The reversible nature of the fluorescence response also demonstrates that the assay responds to the reversible equilibrium between the DFG-in and DFG-out conformations which is triggered by inhibitor binding and dissociation.

FIG. 28

Detection of the binding of imidazole derivatives SB203580 and SKF86002 by ac-p38α. The high affinity ATP-competitive inhibitor of p38α, SKF86002, binds with a K_{d} ˜78 nM (open squares) according to changes in the fluorescence emission of the inhibitor itself upon binding (left axis) while the ratiometric fluorescence of acrylodan (right axis) reports a K_{d} of 721 nM (closed squares) (A). SB203580 shares similar structural moieties (red) with SKF86002 and produced a binding curve with a K_{d} of 15 nM when added to ac-p38α (B). Applicants solved the crystal structure of SB203580 to 2.3 Å and electron density maps of SB203580 (red) and p38α (gray) are contoured to 1σ. Possible hydrogen bonding interactions are highlighted by dotted lines. The pyridine ring of the inhibitor forms an essential hydrogen bond to the hinge region (Met109) of the kinase. The proximal phenyl substituent is perfectly sandwiched between the side chain of Phe169 of the DFG-motif (orange) and the side chain of Tyr35 of the P-loop (green) respectively. The methylsulfinyl substituent phenyl is electron rich and forms energetically favorable π-π interactions with the side chains of Tyr35 and Phe169 and most likely stabilizes the DFG-motif in the “out” conformation. In addition, water molecule W1 bridges a hydrogen bond between N3 of the imidazole of the inhibitor and the backbone carbonyl (red) of Leu167 and is likely to contribute to stabilization of the DFG-out conformation (C). See Example 9 “Detection of Potent ATP-competitive inhibitors/Identifying False Hits in Screens” for further discussion.

FIG. 29

Real-time and endpoint fluorescence measurements using IAEDANS-p38α labeled on the activation loop. IAEDANS emission at 463 nm decreases upon binding of BIRB-796 while ratiometric fluorescence data (R=511 nm/463 nm) increase upon ligand binding to the DFG-out conformation (A). Endpoint equilibrium measurements of ratiometric data following a 360 min incubation were used to reliably obtain the K_{d} of binding of 19.9 nM for BIRB-796 (B). The K_{d} was determined by plotting fluorescence data against a logarithmic scale of inhibitor concentration. Fluorescence traces can also be measured in real-time at a single wavelength (463 nm) to determine various kinetic rate constants (C). The fluorescence decay resulting from the addition of 100 nM BIRB-796 to an equimolar amount of IAEDANS-p38α at time=0 sec was fit (gray lines) to a first-order decay function to determine k_{obs} for binding. Extraction of BIRB-796 from IAEDANS-p38α using a 10-fold excess of unlabeled p38α allowed direct determination of k_{off} which was also fit (gray lines) to a first-order function (D). The data presented above are representative of a typical set of experiments carried out for BIRB-796 using IAEDANS-p38α. The ratio of k_{obs} for dissociation and binding was used to calculate the equilibrium constant (K_{eq}) for BIRB-796 under these experimental conditions (K_{eq}=k_{off}/k_{obs}, on).

FIG. 30

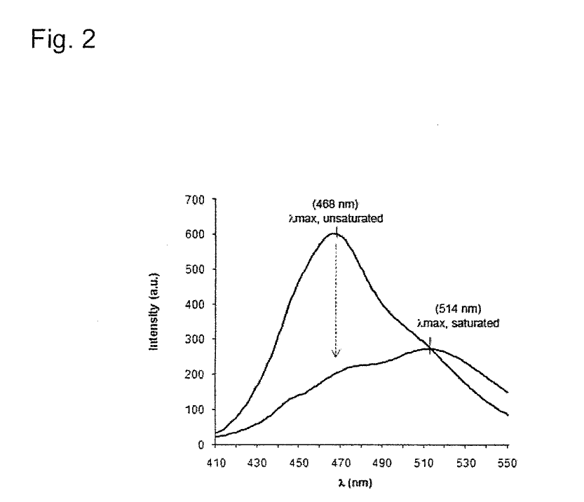

Real-time fluorescence measurements using different p38α-fluorophore conjugates labeled on the activation loop. NBD emission at 535 (A), fluorescein emission at 520 nm (B), pyrene emission at 535 nm (C) and Atto680 emission at 697 nm (D) all decrease upon binding of BIRB-796 (left panels). Since ratiometric fluorescence measurements were not possible with these fluorophores, stable endpoint equilibrium measurements were not ideal for determining the K_{d} of binding. Fluorescence traces could be measured in real-time at the reported single wavelengths to determine various kinetic rate constants. The fluorescence decay resulting from the addition of 100 nM BIRB-796 to an equimolar amount of each fluorescent-labeled p38α conjugate at time=0 sec was fit (gray lines) to a first-order decay function to determine k_{obs} for binding (center panels). Extraction of BIRB-796 from each fluorescent-labeled p38α conjugate using a 10-fold excess of unlabeled p38α allowed direct determination of k_{off} which was also fit (gray lines) to a first-order function. The data presented above are representative of a typical set of experiments carried out for BIRB-796 using each fluorescent-labeled p38α conjugate. The ratio of k_{obs} for dissociation and binding was used to calculate the equilibrium constant (K_{eq}) for BIRB-796 under these conditions (K_{eq}=k_{off}/_{kobs,on}) reported in FIG. 2.

FIG. 31

Measured K_{d} values and SAR of a focused library of pyrazoloureas. The synthesis and characterization of all pyrazolourea derivatives are described in the Supporting Information. All compounds were titrated against ac-p38α (50 nM) over a concentration range of 1 nM-20 μM to generate binding curves using the ratiometric fluorescence change (R=514 nm/468 nm) observed upon binding to the DFG-out conformation of p38α. K_{d} values for each compound were then directly obtained from the binding curves. All reported values represent the mean±s.d. from at least three independent titrations.

FIG. 32

Crystal structure of the Type II hybrid kinase inhibitors 11b, 11e and 11l bound to p38α. Experimental electron densities (ligand red; protein grey) of a 1,4-para quinazoline-pyrazolourea hybrid Type II inhibitor 11b, RL45 (A) and the 1,3-meta quinazoline-pyrazolourea hybrid Type II inhibitors 11e, RL62 (B) and 11l, RL48 (C) at 2.0 Å, 2.3 Å and 2.1 Å resolutions, respectively, are shown (2 Fo−Fc map contoured at 1σ). Hydrogen bonding interactions of the inhibitors with helix C (blue), the DFG-motif (orange) and the hinge region (pink) are shown by red dotted lines. The kinase domain is in the inactive conformation and the pyrazolourea moiety resides in the allosteric site flanked by helix C and the DFG-motif. N1 of the quinazoline makes a direct hydrogen bond to the main chain amide of M109 (hinge region), an interaction commonly formed between anilino-quinazolines and the hinge region of several other protein kinase domains (Blair et al., 2007; Michalczyk et al., 2008). N3 of the quinazoline forms a hydrogen bond with the side chain of the gatekeeper T106. In the p38α-RL45 complex, N2 of the pyrazole forms a water (W1) mediated hydrogen bond to the side chain of D168 (DFG-motif). The central phenyl moiety which links the quinazoline and pyrazolourea scaffolds interacts with the side chain of F169 (DFG-motif) while in the p38α-RL62 and p38α-RL48 complex the secondary amine at the 4-position and the primary amine at the 6-position of the quinazoline core each form a water (W2 and W3) mediated hydrogen bond to the backbone of the DFG-motif. The DFG-motif is pulled closer to the 10-membered ring of the quinazoline and allows the formation of electrostatically favorable edge-to-face interactions (Hunter and Singh, 1991) of both π-electron systems (quinazoline and F169 side chain). The water-mediated hydrogen bonds to the DFG-motif as well as the π-π-interactions in p38α-RL61 and p38α-RL48 most likely stabilize the DFG-out conformation and attribute to the tighter binding of meta-substituted quinazoline-pyrazoloureas. In the p38α-RL45 and p38α-RL48 complexes, the primary amine in the 6-position of the quinazoline is within hydrogen bonding distance to the backbone of V30. The meta-toloyl moiety attached to N1 of the pyrazole flips by 180° in the p38α-RL45 complex when compared to p38α-RL62 complex and reveals a distinct flexibility of the ligand in the vicinity of the allosteric pocket. Measured K_{d} values of a focused library of Type II hybrid inhibitors of the pyrazolourea and quinazoline/quinoline scaffolds (D). All compounds were titrated against ac-p38α (50 nM) over a concentration range of 1 nM-20 μM to generate binding curves for determination of the Kd for each compound. All reported values represent the results from at least three independent titrations. See Example 27 “SAR of Additional Type II Hybrid Inhibitors” for further discussion.

### DETAILED DESCRIPTION OF THE INVENTION

The term “kinase” is well-known in the art and refers to a type of enzyme that transfers phosphate groups from high-energy donor molecules, such as ATP, to specific target molecules such as proteins. Kinases are classified under the enzyme commission (EC) number 2.7. According to the specificity, protein kinases can be subdivided into serine/threonine kinases (EC 2.7.11, e.g. p38α), tyrosine kinases (EC 2.7.10, e.g. the EGFR kinase domain), histidine kinases (EC 2.7.13), aspartic acid/glutamic acid kinases and mixed kinases (EC 2.7.12) which have more than one specificity (e.g. MEK being specific for serine/threonine and tyrosine).

The modified kinases of the present invention are labeled at an amino acid having a free thiol- and/or amino group. Amino acids are defined as organic molecules that have a carboxylic and amino functional group. They are the essential building blocks of proteins. Examples of amino acids having a free thiol group are cysteine, belonging to the 20 standard amino acids, and acetyl-cysteine being a non-standard amino acid rarely occurring in natural amino acid sequences. Standard amino acids having a free amino group are lysine, histidine or arginine and amino acids being aromatic amines, such as tryptophan. Pyrrolysine, 5-hydroxylysine or o-aminotyrosine are non-standard amino acids having a free amino group. The amino acids asparagine and glutamine, although having a free amino group, are not suitable in the present invention as they are not reactive to labeling agents and are thus excluded.

Tryptophan is an aromatic amino acid having an amino group in its indole ring. Aromatic amines are weak bases and thus unprotonated at pH 7. However, they can still be modified using a highly reactive reagent such as an isothiocyanate, sulfonyl chloride or acid halide.

The term “labeled at an amino acid having a free thiol or amino group” describes a kinase having an amino acid which has a free thiol or amino group at the desired position, i.e. in the activation loop, and which is labeled at said amino acid. During labeling, the previously free thiol or amino group is involved in forming the covalent bond between the labeled amino acid and the label according to items (a) and (b). In other words, the term “A kinase labeled at an amino acid having a free thiol or amino group, wherein said amino acid is naturally present or introduced into the activation loop of said kinase, with.” is interchangeably used with the term “A kinase having an amino acid naturally present or introduced into the activation loop of said kinase, wherein said labeling is effected at a free thiol or amino group of said amino acid and said label is . . . ”.

Said amino acid to be labeled is located in the activation loop of the kinase. This means that only kinases having an activation loop or a structure equivalent thereto fall within the present invention. The activation loop is a flexible segment near the entrance to the active site which forms the substrate binding cleft of most kinases and can be phosphorylated on one or more amino acids to provide an important regulatory mechanism throughout the protein kinase superfamily (Johnson and Lewis, 2001; Taylor and Radzio-Andzelm, 1994; Johnson et al., 1996). The activation loop consists of several amino acids which form a loop that is flexible in most kinases which begins with a highly-conserved aspartate-phenylalanine-glycine (DFG) motif in the ATP binding site and extends out between the N- and C-lobes of the kinase. The activation loop is a structural component crucial for enzymatic kinase activity. It is part of the substrate binding cleft and contains several amino acid residues which assist in the recognition of specific substrates and also contains serines, threonines or tyrosines which can be phosphorylated. The conformation of the activation loop is believed to be in dynamic equilibrium between the DFG-in (active kinase) and DFG-out (inactive kinase) conformations. Phosphorylation and/or binding of interaction partners (other proteins or DNA) result in a shift of the equilibrium. In the DFG-in conformation, the aspartate contained in the motif is pointed into the ATP binding site and the adjacent phenylalanine is pointed away from the ATP site and into the adjacent allosteric site. When the conserved DFG motif forming part of the activation loop adopts the in-conformation, ATP-competitive inhibitors (Type I inhibitors) can bind to the kinase. In the DFG-out conformation, the positions of these residues are flipped 180° in orientation. The out-conformation of the activation loop prevents ATP and substrate binding. Compounds causing a conformational change of the activation loop from the in- to the out-conformation are either Type II inhibitors, which bind to the ATP site (hinge region) and extend into the allosteric pocket adjacent to the ATP binding site, or Type III inhibitors, which only bind to the allosteric pocket.

Cysteines which are naturally present in a kinase of interest and are solvent-exposed can be located outside the activation loop or within the activation loop sequence. This equally applies to amino acids having a free amino group.

The modified kinase of the invention is labeled at an amino acid naturally present or introduced into the activation loop. If no suitable amino acid, i.e. one having a free thiol- or amino group, is present in the activation loop, said amino acid can be introduced, i.e. inserted by adding it or by replacing an existing amino acid, by techniques well-known in the art. In any case, it is to be understood for the avoidance of doubt that the amino acid is only labeled after its introduction into the activation loop if it is to be labeled by reaction with labeling reagents. Those techniques comprise site-directed mutagenesis as well as other recombinant, synthetic or semi-synthetic techniques. In case a non-standard amino acid is to be introduced into the kinase, an amino acid stretch containing said amino acid may be chemically synthesized and then connected to the remaining part(s) of the kinase which may have been produced recombinantly or synthetically.

The process of labeling involves incubation of the kinase, e.g. the mutated kinase of the invention (e.g. the kinase with a cysteine introduced in the activation loop), with a thiol- or amino-reactive label under mild conditions resulting in the labeling of said mutated kinase at the desired position in the activation loop. Mild conditions refer to buffer pH (e.g. around pH7 for thiol-reactive probes), ratio of label to kinase, temperature and length of the incubation step (for thiol-reactive probes e.g. 4° C. and overnight in the dark) which are known to the skilled person and provided with instruction manuals of providers of thiol- and amino-reactive probes. Such conditions need to be optimized to slow down the reaction of the chosen thiol- or amino-reactive label to ensure that labeling of said kinase is specific to the desired labeling site. In the case of fluorophore labeling, it is necessary to carry out the incubation in the dark. Increased light exposure results in bleaching of the fluorophore and a less intense fluorescence emission. After labeling, the labeled kinase is preferably concentrated, purified by gel filtration experiments or washed several times with buffer to remove excess unreacted label. The wash buffer is typically the buffer used to store the labeled kinase and may also be the buffer in which the desired measurements are made.

The term “fluorophore” denotes a molecule or functional group within a molecule which absorbs energy such as a photon of a specific wavelength and emits energy, i.e. light at a different (but equally specific) wavelength (fluorescence) immediately upon absorbance (unlike the case in phosphorescence) without the involvement of a chemical reaction (as the case in bioluminescence). Usually the wavelength of the absorbed photon is in the ultraviolet range but can reach also into the infrared range. The wavelength of the emitted light is usually in the visible range. The amount and wavelength of the emitted energy depend primarily on the properties of the fluorophore but may also be influenced by the chemical environment surrounding the fluorophore. A number of fluorophores are sensitive to changes in their environment. This includes changes in the polarity, charge and/or in the conformation of the molecule they are attached to. Fluorescence occurs when a molecule relaxes to its ground state after being electrically excited which, for commonly used fluorescent compounds that emit photons with energies from the UV to near infrared, happens in the range of between 0.5 and 20 nanoseconds.

The term “thiol- or amino-reactive” denotes the property of a compound, e.g. a fluorophore, to specifically react with free thiol- or amino groups. This is due to a functional group present in said compound which directs a specific reaction with a thiol or amino group. These functional groups may be coupled to molecules such as fluorophores, spin labels or isotope-enriched molecules in order to provide specific labels attachable to free thiol- or amino-groups. Examples for thiol-specific compounds are e.g. haloalkyl compounds such as iodoacetamide, maleimides, Hg-Link™ phenylmercury compounds or TS-Link™ reagents (both Invitrogen). Haloalkyl compounds react with thiol or amino groups depending on the pH.

The term “spin label” (SL) denotes a molecule, generally an organic molecule, which possesses an unpaired electron, usually on a nitrogen atom, and has the ability to bind to another molecule. Spin labels are used as tools for probing proteins using EPR spectroscopy. The site-directed spin labeling (SDSL) technique allows one to monitor the conformation and dynamics of a protein. In such examinations, amino acid-specific SLs can be used.

Site-directed spin labeling is a technique for investigating protein local dynamics using electron spin resonance. SDSL is based on the specific reaction of spin labels with amino acids. A spin label built in protein structures can be detected by EPR spectroscopy. In SDSL, sites for attachment of spin labels such as thiol or amino groups, if not naturally present, are introduced into recombinantly expressed proteins by site-directed mutagenesis. Functional groups contained within the spin label determine their specificity. At neutral pH, protein thiol groups specifically react with functional groups such as methanethiosulfonate, maleimide and iodoacetamide, creating a covalent bond with the amino acid cysteine. Spin labels are unique molecular reporters, in that they are paramagnetic, i.e. they contain an unpaired electron. Nitroxide spin labels are widely used for the study of macromolecular structure and dynamics because of their stability and simple EPR signal. The nitroxyl radical (N—O) is usually incorporated into a heterocyclic ring such as pyrrolidine, and the unpaired electron is predominantly localized to the N—O bond. Once incorporated into the protein, a spin label's motions are dictated by its local environment. Because spin labels are exquisitely sensitive to motion, this has profound effects on the EPR spectrum of the spin-label attached to the protein.

The signal arising from an unpaired electron can provide information about the motion, distance, and orientation of unpaired electrons in the sample with respect to each other and to the external magnetic field. For molecules free to move in solution, EPR works on a much faster time-scale than NMR (Nuclear Magnetic Resonance spectroscopy), and so can reveal details of much faster molecular motions, i.e. nanoseconds as opposed to microseconds for NMR. The gyromagnetic ratio of the electron is orders of magnitude larger than of nuclei commonly used in NMR, and so the technique is more sensitive, though it does require spin labeling.

The term “isotope” denotes a chemical species of a chemical element having different atomic mass (mass number) than the most abundant species of said element. Isotopes of an element have nuclei with the same number of protons (the same atomic number) but different numbers of neutrons.

Isotopes suitable for EPR or NMR need to have a nonzero nuclear spin. The most common isotopes currently used are ¹H, ²D, ¹⁵N, ¹³C, and ³¹P.

It is preferred that a kinase labelled with a thiol-reactive spin label is also labelled with an isotope (as described in detail further below).

The term “isotope-enriched” denotes that a compound, e.g. a thiol- or amino-reactive label has been synthesized using or reacted with an isotope so that said isotope is introduced into said compound. The compound may comprise one or more isotopes of one or more different species.

The label has to be positioned so that it does not inhibit the kinase's catalytic activity and does not interfere with its stability. In principle, the assay of the invention does not rely on the measurement of the catalytic activity of the labeled kinase of the invention. However, it is preferable that essentially no interference with the catalytic activity takes place to allow for the comparison of the binding activity of potential inhibitors to the labeled kinase of the invention and the wild-type kinase it is derived from. In the case of a kinase that is isotopically labeled on an amino acid, e.g. a cysteine, and produced by growing host organisms expressing the kinase with isotopically labeled amino acid already incorporated into the sequence, inhibition of the activity or interference with the stability of the kinase is unlikely. On the other hand, care also has to be taken when selecting the position in the activation loop where the label is to be introduced. If no suitable amino acid is present at the position of choice, the amino acid present at said position must be replaced with an amino acid containing a free thiol or amino group. Tests of how to evaluate the activity and stability of a kinase prior to and after replacement of an amino acid are well known to the skilled person and include visual inspection of the purified protein, circular dichroism (CD) spectroscopy, crystallization and structure determination, enzyme activity assays, protein melting curves, differential scanning calorimetry and NMR spectroscopy.

In this regard, no inhibition of the catalytic activity is present if at least 90% of the catalytic activity of the kinase, preferably the wild-type kinase in its active state, are retained, preferably 95%, more preferably 98%. Most preferably, the catalytic activity of the kinase is fully retained. The term “does not inhibit the catalytic activity” is thus, in some embodiments where the catalytic activity amounts to less than 100%, to be equated with and having the meaning of “does not essentially interfere with the catalytic activity”. The catalytic activity can indirectly be determined by comparing the IC50 value of an inhibitor in the labeled kinase of the invention and the unlabeled kinase it is derived from. If the IC50 values are within the same range, i.e. if they do not differ by more than a factor of 5, this indicates that the catalytic activity is essentially the same (and that the modifications to the kinase did not alter inhibitor affinity for the kinase). It is preferred that the labeled kinase of the invention and the unlabeled kinase differ by not more than the factor 4, more preferably by not more than the factor 3, even more preferably by not more than the factor 2. The skilled person is aware that a difference between both IC50 values of up to the factor 5 is well within the usual variance associated with these measurements. Such IC50 values ensure that the catalytic activity of both kinases is essentially the same. Regarding stability, the amino acid introduced does not interfere with the essential intramolecular contacts that ensure structural stability of the protein, so that the kinase can carry out the biological function described herein.

To overcome the drawbacks of presently existing screening methods, the present invention involves a labeling strategy to create fluorescent-tagged kinases which (i) are highly sensitive to the binding of kinase inhibitors, (ii) can be used to measure the kinetics of ligand binding and dissociation in real-time, (iii) can be used to directly measure the Kd of these ligands and (iv) is rapid, robust, reproducible and adaptable to high-throughput screening methods.

As demonstrated in the appended examples, the present invention provides kinases and screening methods using these kinases which enables for screening for specific inhibitors with a reduced effort and material as well as a superior reliability. This is essentially achieved by providing a labeling strategy for a kinase such that the label alters its behavior in reaction to changes in its environment caused e.g. by conformational changes in the activation loop of the kinase.

Besides conventional kinase assays for the screening of modulators of kinase activity, various approaches have recently been developed. However, many of these approaches suffer from major drawbacks. For example, Annis et al. (2004) describe an approach using affinity selection-mass spectrometry (AS-MS). This method is described as suitable for high-throughput screening. However, a size exclusion chromatography step has to be applied prior to the examination of each probe which is time-consuming and requires a lot of material.

De Lorimier et al. (2002) describe a family of biosensors based on bacterial proteins binding to small molecule ligands which were modified and labeled with different environmentally sensitive fluorophores. Upon ligand binding, the fluorophores alter their emission wavelength and/or intensity thus indicating the presence and/or concentration of the specific ligand bound to a probe. However, the labeling of kinases and the use of said kinases in the screening for specific inhibitors is neither disclosed nor suggested.

In a preferred embodiment, the kinase is a serine/threonine kinase or a tyrosine kinase.

In another preferred embodiment, the kinase is p38α, MEK kinase, CSK, an Aurora kinase, GSK-3β, cSrc, EGFR, Abl, DDR1, LCK or another MAPK.

Mitogen-activated protein (MAP) kinases (EC 2.7.11.24) are serine/threonine-specific protein kinases that respond to extracellular stimuli (mitogens) and regulate various cellular activities, such as gene expression, mitosis, differentiation, and cell survival/apoptosis. Extracellular stimuli lead to activation of a MAP kinase via a signaling cascade (“MAPK cascade”) composed of a MAP kinase, MAP kinase kinase (MKK or MAP2K) and MAP kinase kinase kinase (MKKK or MAP3K, EC 2.7.11.25).

A MAP3K that is activated by extracellular stimuli phosphorylates a MAP2K on its serine and/or threonine residues, and then this MAP2K activates a MAP kinase through phosphorylation on its serine and/or tyrosine residues. This MAP kinase signaling cascade has been evolutionarily well-conserved from yeast to mammals.

To date, six distinct groups of MAPKs have been characterized in mammals:
1. extracellular signal-regulated kinases (ERK1, ERK2). The ERK (also known as classical MAP kinases) signaling pathway is preferentially activated in response to growth factors and phorbol ester (a tumor promoter), and regulates cell proliferation and cell differentiation.
2. c-Jun N-terminal kinases (JNKs), (MAPK8, MAPK9, MAPK10), also known as stress-activated protein kinases (SAPKs).
3. p38 isoforms are p38α (MAPK14), p38β (MAPK11), p38γ (MAPK12 or ERK6) and p38δ (MAPK13 or SAPK4). Both JNK and p38 signaling pathways are responsive to stress stimuli, such as cytokines, ultraviolet irradiation, heat shock, and osmotic shock, and are involved in cell differentiation and apoptosis. p38α MAP Kinase (MAPK), also called RK or CSBP, is the mammalian orthologue of the yeast HOG kinase which participates in a signaling cascade controlling cellular responses to cytokines and stress. Similar to the SAPK/JNK pathway, p38 MAP kinase is activated by a variety of cellular stresses including osmotic shock, inflammatory cytokines, lipopolysaccharides (LPS), ultraviolet light and growth factors. p38 MAP kinase is activated by phosphorylation at Thr180 and Tyr182.
4. ERK5 (MAPK7), which has been found recently, is activated both by growth factors and by stress stimuli, and it participates in cell proliferation.
5. ERK3 (MAPK6) and ERK4 (MAPK4) are structurally related atypical MAPKs which possess an SEG (serine-glutamic acid-glycine) motif in the activation loop and display major differences only in the C-terminal extension.
6. ERK7/8 (MAPK15) are the most recently discovered members of the MAPK family and behave similar to ERK3/4.

Mitogen-activated protein kinase kinase forms a family of kinases which phosphorylates mitogen-activated protein kinase. They are also known as MAP2K and classified as EC 2.7.12.2. Seven genes exist. These encode MAP2K1 (MEK1), MAP2K2 (MEK2), MAP2K3 (MKK3), MAP2K4 (MKK4), MAP2K5 (MKKS), MAP2K6 (aka MKK6), MAP2K7 (MKK7). The activators of p38 (MKK3 and MKK4), JNK (MKK4), and ERK (MEK1 and MEK2) define independent MAP kinase signal transduction pathways.

Aurora kinases A (also known as Aurora, Aurora-2, AIK, AIR-1, AIRK1, AYK1, BTAK, Eg2, MmIAK1, ARK1 and STK15), B (also known as Aurora-1, AIM-1, AIK2, AIR-2, AIRK-2, ARK2, IAL-1 and STK12) and C (also known as AIK3) participate in several biological processes, including cytokinesis and dysregulated chromosome segregation. These important regulators of mitosis are over-expressed in diverse solid tumors. One member of this family of serine/threonine kinases, human Aurora A, has been proposed as a drug target in pancreatic cancer. The recent determination of the three-dimensional structure of Aurora A has shown that Aurora kinases exhibit unique conformations around the activation loop region. This property has boosted the search and development of inhibitors of Aurora kinases, which might also function as novel anti-oncogenic agents.

Glycogen synthase kinase 3 (GSK-3) is a serine/threonine protein kinase which in addition to the serine/threonine kinase activity has the unique ability to auto-phosphorylate on tyrosine residues. The phosphorylation of target proteins by GSK-3 usually inhibits their activity (as in the case of glycogen synthase and NFAT). GSK-3 is unusual among the kinases in that it usually requires a “priming kinase” to first phosphorylate a target protein and only then can GSK-3 additionally phosphorylate the target protein. In mammals GSK-3 is encoded by two known genes, GSK-3 alpha and beta. Aside from roles in pattern formation and cell proliferation during embryonic development, there is recent evidence for a role in tumor formation via regulation of cell division and apoptosis. Human glycogen synthase kinase-3 beta (GSK3β) is also associated with several pathophysiological conditions such as obesity, diabetes, Alzheimer's disease and bipolar disorder.

The Src family of proto-oncogenic tyrosine kinases transmit integrin-dependent signals central to cell movement and proliferation. The Src family includes nine members: Src, Lck, Hck, Fyn, Blk, Lyn, Fgr, Yes, and Yrk. These kinases have been instrumental to the modern understanding of cancer as a disease with disregulated cell growth and division. The cSrc proto-oncogene codes for the cSrc tyrosine kinase. Besides its kinase domain, cSrc is further comprised of an SH2 domain and an SH3 domain, which act as adaptor proteins for the formation of multi-enzyme complexes with the Src kinase domain. These domains are also involved in the auto-inhibition of the cSrc kinase domain. Mutations in this gene could be involved in the malignant progression of cancer cells. This protein specifically phosphorylates Tyr-504 residue on human leukocyte-specific protein tyrosine kinase (Lck), which acts as a negative regulatory site. It may also act on the Lyn and Fyn kinases.

Leukocyte-specific protein tyrosine kinase (Lck) is a protein that is found inside lymphocytes such as T-cells. Lck is a tyrosine kinase which phosphorylates tyrosine residues of certain proteins involved in the intracellular signaling pathways of lymphocytes. The N-terminal tail of Lck is myristoylated and palmitoylated, which tethers the protein to the plasma membrane of the cell. The protein furthermore contains an SH3 domain, an SH2 domain and in the C-terminal part the tyrosine kinase domain. The tyrosine phosphorylation cascade initiated by Lck culminates in the intracellular mobilization of calcium (Ca²⁺) ions and activation of important signaling cascades within the lymphocyte. These include the Ras-MEK-ERK pathway, which goes on to activate certain transcription factors such as NFAT, NFκB, and AP-1 which then regulate the production of a plethora of gene products, most notably, cytokines such as Interleukin-2 that promote long-term proliferation and differentiation of the activated lymphocytes. Aberrant expression of Lck has been associated with thymic tumors, T-cell leukemia and colon cancers.

The catalytic activity of the Src family of tyrosine kinases is suppressed by phosphorylation on a tyrosine residue located near the C terminus (Tyr 527 in cSrc), which is catalyzed by C-terminal Src Kinase (Csk). Given the promiscuity of most tyrosine kinases, it is remarkable that the C-terminal tails of the Src family kinases are the only known targets of Csk. Interactions between Csk and cSrc, most likely representative for src kinases, position the C-terminal tail of cSrc at the edge of the active site of Csk. Csk cannot phosphorylate substrates that lack this docking mechanism because the conventional substrate binding site used by most tyrosine kinases to recognize substrates is destabilized in Csk by a deletion in the activation loop (Levinson, 2008).

The epidermal growth factor receptor (EGFR; ErbB-1; HER1 in humans) is the cell-surface receptor for members of the epidermal growth factor family (EGF-family) of extracellular protein ligands. The epidermal growth factor receptor is a member of the ErbB family of receptors, a subfamily of four closely related receptor tyrosine kinases: EGFR (ErbB-1), HER2/c-neu (ErbB-2), Her 3 (ErbB-3) and Her 4 (ErbB-4). Active EGFR occurs as a dimer. EGFR dimerization is induced by ligand binding to the extracellular receptor domain and stimulates its intrinsic intracellular protein-tyrosine kinase activity. As a result, autophosphorylation of several tyrosine residues in the C-terminal (intracellular) domain of EGFR occurs. This autophosphorylation elicits downstream activation and signaling by several other proteins that associate with the phosphorylated tyrosines through their own phosphotyrosine-binding SH2 domains. The kinase domain of EGFR can also cross-phosphorylate tyrosine residues of other receptors it is aggregated with, and can itself be activated in that manner. The EGFR signaling cascade activates several downstream signaling proteins which then initiate several signal transduction cascades, principally the MAPK, Akt and JNK pathways, leading to DNA synthesis and cell proliferation. Such pathways modulate phenotypes such as cell migration, adhesion, and proliferation. Mutations that lead to EGFR overexpression (known as upregulation) or overactivity have been associated with a number of cancers. Consequently, mutations of EGFR have been identified in several types of cancer, and it is the target of an expanding class of anticancer therapies.

The ABL1-protooncogene encodes a cytoplasmic and nuclear protein tyrosine kinase that has been implicated in processes of cell differentiation, cell division, cell adhesion and stress response. The activity of c-Abl protein is negatively regulated by its SH3 domain. A genetic deletion of the SH3 domain turns ABL1 into an oncogene. This genetic deletion, caused by the (9;22) gene translocation results in the head-to-tail fusion of the BCR (MIM:151410) and ABL1 genes present in many cases of chronic myelogeneous leukemia. The DNA-binding activity of the ubiquitously expressed ABL1 tyrosine kinase is regulated by CDC2-mediated phosphorylation, suggesting a cell cycle function for ABL1.

Discoidin domain receptor family, member 1, also known as DDR1 or CD167a (cluster of differentiation 167a), is a receptor tyrosine kinase (RTK) that is widely expressed in normal and transformed epithelial cells and is activated by various types of collagen. This protein belongs to a subfamily of tyrosine kinase receptors with a homology region similar to the Dictyostelium discoideum protein discoidin I in their extracellular domain. Its autophosphorylation is achieved by all collagens so far tested (type I to type VI). In situ studies and Northern-blot analysis showed that expression of this encoded protein is restricted to epithelial cells, particularly in the kidney, lung, gastrointestinal tract, and brain. In addition, this protein is significantly over-expressed in several human tumors from breast, ovarian, esophageal, and pediatric brain.

The kinases described above are preferred embodiments because all of them are involved in the development of diseases such as cancer for which at present no suitable cure is available or an improved treatment regimen is desired.

Using p38α, a kinase for which structural information was available, the present inventors demonstrated the applicability of the labeled kinase of the invention for screening purposes. Unexpectedly, the kinase could be prepared for labeling with a minimum of effort but also the labeled kinase exerted the desired properties, i.e. the introduced label proved suitable for the detection of conformational changes induced by binding of a specific inhibitor, in this case the known inhibitor BIRB-796 and several smaller BIRB-796 analogs.

A further kinase which in its labeled form according to the invention can be applied for screening for specific inhibitors is cSrc. Both Type II and Type III inhibitors for cSrc were identified and their pharmacological profile could be refined to obtain more potent inhibitors, as detailed in the examples.

In summary, the applicability of the labeling principle of the present invention has been shown by the present inventors to work with various classes of kinases, including tyrosine and serine/threonine kinases.

In another preferred embodiment, the amino acid having a free thiol or amino group is cysteine, lysine, arginine or histidine.

Cysteine has a free thiol group, whereas lysine, arginine or histidine each possess at least one free amino group.

In another preferred embodiment, one or more solvent-exposed cysteines present outside the activation loop are deleted or replaced.

If more than one amino acid having a free thiol or amino group is present in a kinase of interest, specific labeling of the amino acid in the activation loop may not be possible. Therefore, as discussed above, amino acids having a free thiol or amino group should be deleted or replaced with another amino acid not having a free thiol or amino group if they are predicted or shown to be solvent-exposed. Cysteines which are naturally present in a kinase of interest and are solvent-exposed can be located outside the activation loop, in which case they should be deleted or replaced with another amino acid not having a free thiol group. This equally applies to amino acids having a free amino group which should then be replaced with an amino acid not having a reactive free amino group. In case that one or more amino acids having a free amino group is already present in the activation loop, amino acids having a free amino group and present in the activation loop in addition to the amino acid to be labeled, should be replaced or deleted, whichever of these mutations to the kinase does not inhibit its catalytic activity or interfere with its stability.

The term “solvent-exposed” refers to the position of an amino acid in the context of the three dimensional structure of the protein of which it is a part. Amino acids buried within the protein body are completely surrounded by other amino acids thus do not have any contact with the solvent. In contrast, solvent-exposed amino acids are partially or fully exposed to the surrounding solvent and are thus accessible to chemicals potentially able to modify them. This applies e.g. to thiol- or amino-reactive labels used in the present invention which can react with solvent-exposed amino acids having a free thiol- or amino-group.

The term “delete” refers to excision of an amino acid without replacing it with another amino acid whereas the term “replace” refers to the substitution of an amino acid with another amino acid. If an amino acid is replaced with another amino acid or deleted, the amino acid to be replaced or to be deleted is preferably chosen such that the amino acid deleted or replaced does not result in a kinase with inhibited catalytic activity and does not interfere with the stability of the resulting kinase.

In a more preferred embodiment, the kinase is p38α and a cysteine is introduced at position 172 of SEQ ID NO: 1 and preferably the cysteines at positions 119 and 162 of SEQ ID NO: 1 are replaced with another amino acid not having a free thiol group such as serine. Said cysteine introduced at position 172 of SEQ ID NO: 1 is the amino acid to be labeled.

In another more preferred embodiment, the kinase is cSrc and a cysteine is introduced at position 157 of SEQ ID NO: 2 (position 407 in wild-type cSrc) and preferably the cysteines at position 27, 233 and 246 of SEQ ID NO: 2 (positions 277, 483 and 496 in wild-type cSrc) are replaced with another amino acid. Said cysteine introduced at position 157 of SEQ ID NO: 2 is the amino acid to be labeled.

In general, amino acid replacements should be conservative. For cysteine, this means that it is preferably replaced with serine. In general, replacements of amino acids with different amino acids may be evaluated of whether they are conservative using the PAM250 Scoring matrix. The matrix is frequently used to score aligned peptide sequences to determine the similarity of those sequences (Pearson, 1990).

As described above, if not naturally present, an amino acid having a free thiol- or amino group has to be introduced into the activation loop of a kinase. In the case of p38α, structural studies were carried out using the available crystal structures for p38α in both the activated (DFG-in) and inactivated (DFG-out) state. P38α does not possess a cysteine in the activation loop. The above structural studies suggested that a replacement of alanine with a cysteine at position 172, which is located in the activation loop, would not influence the catalytic activity or stability of the kinase.

The same studies revealed that two cysteines at positions 119 and 162 of SEQ ID NO: 1 are both solvent-exposed. To avoid potential interferences of the signals recorded for two additional cysteines not located in the activation loop, these two cysteines are preferably replaced with another amino acid, preferably with an amino acid similar in size and structure, such as serine.

If a kinase homologous to p38α is used, the position of the amino acid to be replaced with cysteine may correspond to position 172 in SEQ ID NO: 1. To determine which position in a kinase corresponds to position 172 in SEQ ID NO: 1, sequence alignments of SEQ ID NO: 1 with the used kinase can be effected, e.g. using publicly available programs such as CLUSTALW (Larkin et al., 2007).

In another preferred embodiment, the thiol- or amino-reactive fluorophore is an environmentally sensitive di-substituted naphthalene compound of which one of the two substituents is a thiol- or amino-reactive moiety. The term “environmentally sensitive” denotes the sensitivity of the fluorophore to the conditions in its environment which is expressed in an alteration in its fluorescence emission at one or more wavelengths or in its complete emission spectrum. Conditions causing such alteration are e.g. changes in the polarity or conformational changes in the activation loop.

The above types of fluorophores typically exhibit changes in both intensity and a shift in the emission wavelength depending on the polarity of the surrounding environment. Examples of this class of fluorophores include 6-acryloyl-2-dimethylaminonaphthalene (Acrylodan), 6-bromoacetyl-2-dimethylamino-naphthalenebadan (Badan), 2-(4′-(iodoacetamido)anilino)naphthalene-6-sulfonic acid, sodium salt (IAANS), 2-(4′-maleimidylanilino)naphthalene-6-sulfonic acid, sodium salt (MIANS), 5-((((2-iodoacetyl)amino)ethyl)amino)naphthalene-1-sulfonic acid (1,5-IAEDANS) and 5-dimethylaminonaphthalene-1-sulfonyl aziridine (dansyl aziridine) or a derivative thereof.

Other fluorophores which may be used due to their environmental sensitivity are coumarin-based compounds, benzoxadiazole-based compounds, dapoxyl-based compounds, biocytin-based compounds, fluorescein, sulfonated rhodamine-based compounds such as AlexaFluor dyes (Molecular Probes), Atto fluorophores (Atto Technology) or Lucifer Yellow. Coumarin-based fluorophores are moderately sensitive to environment and 7-diethylamino-3-(4′-maleimidylphenyl)-4-methylcoumarin (CPM) is an example. Benzoxadiazole fluorophores are also commonly used for forming protein-fluorophore conjugates and have a strong environmental dependence with 7-fluorobenz-2-oxa-1,3-diazole-4-sulfonamide (ABD-F) and N-((2-(iodoacetoxy)ethyl)-N-methyl)amino-7-nitrobenz-2-oxa-1,3-diazole ester (IANBD) as examples. PyMPO maleimide (for thiols) or succinimide ester (for amines) and various other dapoxyl dyes have good absorptivity and exceptionally high environmental sensitivity. Examples are 1-(2-maleimidylethyl)-4-(5-(4-methoxyphenyl)oxazol-2-yl)pyridinium methanesulfonate (PyMPO-maleimide), 1-(3-(succinimidyloxycarbonyl)benzyl)-4-(5-(4-methoxyphenyl)oxazol-2-yl)pyridinium bromide (PyMPO-succinimidyl ester) and Dapoxyl (2-bromoacetamidoethyl) sulphonamide. However, due to their longer more flexible structures, these probes may effect activation loop movement depending on the labeling site chosen. As demonstrated in the appended examples, pyrene could be used as a label but did not prove to be preferable. The applicability of the above substances depends on the individual kinase and the position of the amino acid to be labeled so that they can in principle be applied as labels as well, even if in some cases they may cause a reduced sensitivity in the methods of the invention. Matching the above substances with a suitable kinase can be performed by the skilled artisan using routine procedures in combination with the teachings of this invention.

In general, any fluorophore can be used as long as it does not inhibit the catalytic activity or interfere with the stability of the kinase. This means that the fluorophore is preferably not bulky or extended.

In a further preferred embodiment, the thiol-reactive spin-label is a nitroxide radical.

The dominant method for site-specifically labeling protein sequences with a spin-label is the reaction between methanethiosulfonate spin label and cysteine, to give the spin-labeled cysteine side chain, CYS-SL:

MeS(O)₂SSR+R′SH--->R′SSR+MeS(O)2SH

where R is the nitroxide group and R′SH is a protein with a cysteine sulfhydryl, and R′SSR is the spin-labeled protein. The cysteines for labeling are placed in the desired sequence position either through solid-phase techniques or through standard recombinant DNA techniques.

The present invention furthermore relates to a method of screening for kinase inhibitors comprising (a) providing a fluorescently or spin-labeled or isotope-labeled kinase according to the invention; (b) contacting said fluorescently or spin-labeled or isotope-labeled kinase with a candidate inhibitor; (c) recording the fluorescence emission signal at one or more wavelengths or a spectrum of said fluorescently labeled kinase of step (a) and step (b) upon excitation; or (c)′ recording the electron paramagnetic resonance (EPR) or nuclear magnetic resonance (NMR) spectra of said spin-labeled or isotope-labeled kinase of step (a) and step (b); and (d) comparing the fluorescence emission signal at one or more wavelengths or the spectra recorded in step (c) or the EPR or NMR spectra recorded in step (c)′; wherein a difference in the fluorescence intensity at least one wavelength, preferably at the emission maximum and/or a shift in the fluorescence emission wavelength in the spectra of said fluorescently labeled kinase obtained in step (c), or an alteration in the EPR or NMR spectra of said spin-labeled or isotope-labeled kinase obtained in step (c)′ indicates that the candidate inhibitor is a kinase inhibitor.

Kinase inhibitors are substances capable of inhibiting the activity of kinases. They can more specifically inhibit the action of a single kinase, e.g. if they are allosteric inhibitors (Type III) or those binding to the allosteric site adjacent to the ATP-binding site and reaching into the ATP-binding pocket (Type II). Alternatively, an inhibitor can inhibit the action of a number of protein kinases, which is particularly the case if it binds exclusively to the ATP-binding pocket (Type I), which is very conserved among protein kinases.

A candidate inhibitor may belong to different classes of compounds such as small organic or inorganic molecules, proteins or peptides, nucleic acids such as DNA or RNA. Such compounds can be present in molecule libraries or designed from scratch.

Small molecules according to the present invention comprise molecules with a molecular weight of up to 2000 Da, preferably up to 1500 Da, more preferably up to 1000 Da and most preferably up to 500 Da.

Recording the fluorescence emission signal at one or more wavelengths or a spectrum is usually accomplished using a fluorescence spectrometer or fluorimeter. Fluorescence spectroscopy or fluorimetry or spectrofluorimetry is a type of electromagnetic spectroscopy which analyzes fluorescence, or other emitted light, from a sample. It involves using a beam of light, usually ultraviolet light, that excites the electrons in certain molecules and causes them to emit light of a lower energy upon relaxation, typically, but not necessarily, visible light.

Two general types of instruments exist which can both be employed in the method of the invention: Filter fluorimeters use filters to isolate the incident light and fluorescent light, whereas spectrofluorimeters use diffraction grating monochromators to isolate the incident light and fluorescent light. Both types utilize the following scheme: The light from an excitation source passes through a filter or monochromator and strikes the sample. A proportion of the incident light is absorbed by the sample, and some of the molecules in the sample fluoresce. The fluorescent light is emitted in all directions. Some of this fluorescent light passes through a second filter or monochromator and reaches a detector, which is usually placed at 90° to the incident light beam to minimize the risk of transmitted or reflected incident light reaching the detector. Various light sources may be used as excitation sources, including lasers, photodiodes, and lamps; xenon and mercury vapor lamps in particular. The detector can either be single-channeled or multi-channeled. The single-channeled detector can only detect the intensity of one wavelength at a time, while the multi-channeled detects the intensity at all wavelengths simultaneously, making the emission monochromator or filter unnecessary. The different types of detectors have both advantages and disadvantages. The most versatile fluorimeters with dual monochromators and a continuous excitation light source can record both an excitation spectrum and a fluorescence spectrum. When measuring fluorescence spectra, the wavelength of the excitation light is kept constant, preferably at a wavelength of high absorption, and the emission monochromator scans the spectrum. For measuring excitation spectra, the wavelength passing though the emission filter or monochromator is kept constant and the excitation monochromator is scanning. The excitation spectrum generally is identical to the absorption spectrum as the fluorescence intensity is proportional to the absorption (for reviews see Rendell, 1987; Sharma and Schulman, 1999; Gauglitz and Vo-Dinh, 2003; Lakowicz, 1999).

Nuclear magnetic resonance (NMR) is a physical phenomenon based upon the quantum mechanical magnetic properties of the nucleus of an atom. All nuclei that contain odd numbers of protons or neutrons have an intrinsic magnetic moment and angular momentum. The most commonly measured nuclei are hydrogen (¹H) (the most receptive isotope at natural abundance) and carbon (¹³C), although nuclei from isotopes of many other elements (e.g. ¹¹³Cd, ¹⁵N, ¹⁴N ¹⁹F, ³¹P, ¹⁷O, ²⁹Si, ¹⁰B, ¹¹B, ²³Na, ³⁵Cl, ¹⁹⁵Pt) can also be observed. NMR resonant frequencies for a particular substance are directly proportional to the strength of the applied magnetic field, in accordance with the equation for the Larmor precession frequency. NMR measures magnetic nuclei by aligning them with an applied constant magnetic field and perturbing this alignment using an alternating magnetic field, those fields being orthogonal. The resulting response to the perturbing magnetic field is the phenomenon that is exploited in NMR spectroscopy and magnetic resonance imaging, which use very powerful applied magnetic fields in order to achieve high spectral resolution, details of which are described by the chemical shift and the Zeeman Effect.

In the present invention, a suitable amino acid in the activation loop can be labeled with an isotope or thiol/amine-reactive small molecule containing enriched isotopes. In this case, the only signal comes from the enriched molecule on the activation loop, which is sensitive to protein conformation depending on the labeling site chosen.

Preferred isotopes are ¹³C, ¹⁵N, etc. which can be measured as 1D or 2D NMR spectra. Changes in protein conformation, e.g. due to the binding of an inhibitor will result in a shift of the NMR chemical shift(s) corresponding to the label.

Electron paramagnetic resonance (EPR) or electron spin resonance (ESR) spectroscopy, as has been briefly described above, is a technique for studying chemical species that have one or more unpaired electrons, such as organic and inorganic free radicals or inorganic complexes possessing a transition metal ion. The basic physical concepts of EPR are analogous to those of nuclear magnetic resonance (NMR), but it is electron spins that are excited instead of spins of atomic nuclei. Because most stable molecules have all their electrons paired, the EPR technique is less widely used than NMR. However, this limitation to paramagnetic species also means that the EPR technique is one of great specificity, since ordinary chemical solvents and matrices do not give rise to EPR spectra.

The EPR technique utilizes spin-labels. In this case, the kinase, to be examined is expressed in bacteria or other suitable host cells in the presence of an isotope such as ¹³C and ¹⁵N resulting in the incorporation of these isotopes throughout the entire protein as it is expressed. After purification of the isotope enriched protein, a spin label is attached to the activation loop as described above. In this case, 2D NMR spectra of the isotopes in the protein are recorded. As the activation loop and spin label change conformation, the spin label will induce a change in some of the protein signals coming from the incorporated isotopes which come into closer contact with the activation loop or spin label as inhibitors bind. Peaks would become broader as the spin label approaches.

Different EPR spectra or fluorescence emission signals at one or more wavelengths, preferably at the emission maximum, or different fluorescence emission spectra obtained in step (c) or (c)′ indicate a conformational change in the kinase caused by binding of the candidate compound. This is due to the fact that binding of a compound to the allosteric site adjacent to the ATP-binding pocket, and in some cases to the ATP-binding pocket itself, results in a perturbation of the DFG motif, a conformational change in the activation loop, a polarity change and/or a change in the interaction of free electrons in an attached spin-label with the nuclei of adjacent atoms. Upon comparison of the EPR or NMR spectra or the fluorescence emission, the present method reveals whether a candidate compound qualifies as a suitable kinase inhibitor, e.g. not only a high-affinity inhibitor but also one which specifically inhibits the activity of one kinase. The data recorded for the kinase without a candidate inhibitor and those recorded for the kinase having been contacted with said candidate inhibitor are compared. In case of fluorescence emission signal either the signal at one or more specific wavelengths can be recorded and compared enabling for a detection of a change in the intensity of the signal at the particular wavelength(s). Alternatively, a complete spectrum can be recorded and compared enabling also for the observation of changes in the maximum emission wavelength.

Preferably, said method is effected in high-throughput format. High-throughput assays, independently of being biochemical, cellular or other assays, generally may be performed in wells of microtiter plates, wherein each plate may contain 96, 384 or 1536 wells. Handling of the plates, including incubation at temperatures other than ambient temperature, and bringing into contact with test compounds, in this case putative inhibitors, with the assay mixture containing the labelled kinase of the invention is preferably effected by one or more computer-controlled robotic systems including pipetting devices. In case large libraries of test compounds are to be screened and/or screening is to be effected within short time, mixtures of, for example 10, 20, 30, 40, 50 or 100 test compounds may be added to each well. In case a well exhibits inhibitory activity, said mixture of test inhibitors may be de-convoluted to identify the one or more test inhibitors in said mixture giving rise to said activity.

Alternatively, only one test inhibitor may be added to a well, wherein each test inhibitor is applied in different concentrations. For example, the test inhibitor may be tested in two, three or four wells in different concentrations. In this initial screening, the concentrations may cover a broad range, e.g. from 10 nM to 10 μM. The initial screening serves to find hits, i.e. test inhibitors exerting inhibiting activity at least one concentration, preferably two, more preferably all concentrations applied, wherein the hit is more promising if the concentration at which an inhibitory activity can be detected is in the lower range. This alternative serves as one preferred embodiment in accordance with the invention.

Test inhibitors considered as a hit can then be further examined using an even wider range of inhibitor concentrations, e.g. 10 nM to 20 μM. The method applied for these measurements is described in the following.

The present invention furthermore relates to a method of determining the kinetics of ligand binding and/or of association or dissociation of a kinase inhibitor comprising (a) contacting a fluorescently labeled kinase according to the invention with different concentrations of an inhibitor; or (a)′ contacting a fluorescently labeled kinase according to the invention bound to an inhibitor with different concentrations of unlabelled kinase; (b) recording the fluorescence emission signal at one or more wavelengths or a spectrum of said fluorescently labeled kinase for each concentration of inhibitor and/or unlabeled kinase upon excitation; (c) determining the rate constant for each concentration from the fluorescence emission signals at one or more wavelengths or the spectra recorded in step (b) or (c1) determining the K_{d} from the fluorescence emission signal at one or more wavelengths or the spectra recorded in step (b) for each concentration of inhibitor; or (c2) determining the Kₐ or inverse K_{d} from the fluorescence emission signal at one or more wavelengths or the spectra recorded in step (b) for each concentration of unlabelled kinase; (d) directly determining the kₒₙ and/or extrapolating the k_{off} from the rate constants determined in step (c) from the signals or spectra for the different concentrations of inhibitor obtained in step (b); or (d)′ directly determining the k_{off} and/or extrapolating the kₒₙ from the rate constants determined in step (c) from the signals or spectra for the different concentrations of unlabelled kinase obtained in step (b); and optionally (e) calculating the K_{d} and/or Kₐ from kₒₙ and k_{off} obtained in step (d) or (d)′.

By contacting a labeled kinase with different concentrations of an inhibitor, and subsequently determining the fluorescence emission for each concentration applied, the binding affinity of an inhibitor can be measured. For each concentration, the ratio of bound and unbound inhibitor will be different, reflecting the increasing concentration of inhibitor but also the specific binding affinity of said inhibitor to said kinase.

The opposite approach can be followed by titrating a labeled kinase containing a bound inhibitor with unlabeled kinase with no inhibitor bound.

In chemical kinetics, a rate constant k quantifies the speed of a chemical reaction. For a chemical reaction where substance A and B are reacting to produce C, the reaction rate has the form:

$$\frac{d [ C ]}{d t} = {{k ( T ) [ A ]}^{m} [ B ]}^{n}$$

Wherein k(T) is the reaction rate constant that depends on temperature.

[A] and [B] are the concentrations of substances A and B, respectively, in moles per volume of solution assuming the reaction is taking place throughout the volume of the solution.

The exponents m and n are the orders and depend on the reaction mechanism. They can be determined experimentally.

A single-step reaction can also be described as:

$$\frac{d [ C ]}{d t} = A {{{e}^{\frac{- {E}_{a}}{R T}} [ A ]}^{m} [ B ]}^{n}$$

Eₐ is the activation energy and R is the Gas constant. Since at temperature T the molecules have energies according to a Boltzmann distribution, one can expect the proportion of collisions with energy greater than Eₐ to vary with e^{-Ea/RT}. A is the pre-exponential factor or frequency factor.

kₒₙ and k_{off} are constants that describe non-covalent equilibrium binding. When a ligand interacts with a receptor, or when a substrate interacts with an enzyme, the binding follows the law of mass action.

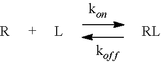

In this equation R is the concentration of free receptor, L is the concentration of free ligand, and RL is the concentration of receptor-ligand complex. In the case of enzyme kinetics, R is the enzyme, or in this case a protein kinase, and L is the substrate, or in this case a candidate or known inhibitor. The association rate constant kₒₙ is expressed in units of M⁻¹sec⁻¹. The rate of RL formation equals R×L×kₒₙ. The dissociation rate constant koff is expressed in units of sec-¹. The rate of RL dissociation equals RL×k_{off}. At equilibrium, the backward (dissociation) reaction equals the forward (association) reaction. Binding studies measure specific binding, which is a measure of RL. Enzyme kinetic assays assess enzyme velocity, which is proportional to RL, the concentration of enzyme-substrate complexes.

$$R L = R \cdot L \cdot \frac{{k}_{on}}{{k}_{off}}$$

The equilibrium dissociation constant, Kd is expressed in molar units and defined to equal koff/kon to arrive at

$$R L = R \cdot L \cdot \frac{{k}_{on}}{{k}_{off}} = \frac{R \cdot L}{{K}_{d}}$$

The dissociation constant (K_{d}) corresponds to the concentration of ligand (L) at which the binding site on a particular protein is half occupied, i.e. the concentration of ligand, at which the concentration of protein with ligand bound (RL), equals the concentration of protein with no ligand bound (R). The smaller the dissociation constant, the more tightly bound the ligand is, or the higher the affinity between ligand and protein.

Accordingly, the association constant Kₐ, also called inverse Kd, is defined as 1/k_{d}. The dissociation constant for a particular ligand-protein interaction can change significantly with solution conditions (e.g. temperature, pH and salt concentration).

Depending on which sequence of steps is followed in the above method of the invention, the K_{d} or Kₐ can be measured directly or indirectly.

For directly measuring the K_{d} or the Kₐ, respectively, step (c1) or (c2) which is the last step for this type of measurement follows step (b). This type of measurement is called endpoint measurement and also illustrated in the appended examples. Unlike for indirectly determining K_{d} or Kₐ through calculation using rate constants, the final fluorescence emission at equilibrium is measured rather than the fluorescence change over time. These measurements can be used to generate a binding curve using different inhibitor concentrations (for determining K_{d}) or concentrations of unlabelled kinase (for determining Kₐ). From these curves, K_{d} or Kₐ can be obtained directly.

For indirectly obtaining K_{d} or Kₐ, the rate constants from the fluorescence emission signal at one or more wavelengths or the spectra recorded in step (b) have to be determined for each concentration as done in step (c). Depending the type of titration, i.e. titration of labeled kinase with inhibitor or titration of labeled kinase bound to inhibitor with unlabeled kinase, either kₒₙ or k_{off} can be determined directly from the measured rate constants. For determining kₒₙ, step (d) is applied which also enables for extrapolation of k_{off}. Accordingly, step (d) is applied for directly determining k_{off} which in turn enables for extrapolation of kₒₙ. From kₒₙ and/or k_{off} obtained in steps (d) or (d)′, the K_{d} and/or Kₐ can be calculated according to the equations discussed above.

The above method may also be applied in high-throughput screens. If a compound exerting inhibitory activity on a kinase has been identified, e.g. using the method of screening for kinase inhibitors of the invention, the present method can be used to further characterize said inhibitor. For example, the high-throughput format can be used to determine the Ka or Kd from the fluorescence emission signal at one or more wavelengths for multiple different concentrations of inhibitors (variant (a)) or, unlabelled kinase (variant (b)). Concentration ranges to be tested reach for example from 10 nM to 20 μM such that repeating series of 1, 2 and 5 (i.e. 10, 20, 50, 100, 200, 500 nM, etc.) between the concentrations assessed.

In a different embodiment, the present invention relates to a method of determining the dissociation or association of a kinase inhibitor comprising (a) contacting a spin-labeled or isotope-labeled kinase according to the invention with different concentrations of an inhibitor; or (a)′ contacting a spin-labeled or isotope-labeled kinase according to the invention bound to an inhibitor with different concentrations of unlabelled kinase; (b) recording the EPR or NMR spectrum of said spin-labeled or isotope-labeled kinase for each concentration of inhibitor and/or unlabelled kinase; and (c) determining the K_{d} from the EPR or NMR spectra recorded in step (b) for the different concentrations of inhibitor; or (c)′ determining the Kₐ from the EPR or NMR spectra recorded in step (b) for the different concentrations of unlabeled kinase.

Similar to the method disclosed further above relating to determining the kinetic constants using fluorescently labeled kinase, the present method allows for the direct determination of the association or dissociation constants for the reaction of a kinase and an inhibitor. Unlike for fluorescently labeled kinases, the instrumental limitations and time required to collect NMR and EPR measurements are, in most cases, not compatible with the fast time scale of inhibitor binding and do not allow the direct determination of kₒₙ or k_{off}. Determinations for compounds which require several hours to bind to the kinase may also be possible.

The methods of the invention relating to determining kinetic data can also be applied to a high-throughput format. For example, a potential inhibitor identified with the screening method of the invention described above can be further characterized in that different concentrations of said inhibitor are applied to the kinase to determine the K_{d}. Suitable but not limiting concentration ranges for the inhibitor are between 10 nM and 20 μM.

More focused concentration ranges applied in the high-throughput format may serve to obtain more sensitive Kd measurements, e.g. with the cuvette approach and real-time kinetics measurements as done in the appended examples, by determining kₒₙ and k_{off}.

The present invention furthermore relates to a method of generating mutated kinases suitable for the screening of kinase inhibitors comprising (a) replacing solvent exposed amino acids having a free thiol or amino group, if any, present in a kinase of interest outside the activation loop or amino acids having a free thiol or amino group at an unsuitable position within the activation loop with an amino acid not having a free thiol or amino group; (b) mutating an amino acid in the activation loop of said kinase of interest to an amino acid having a free thiol or amino group if no amino acid having a free thiol or amino group is present in the activation loop; (c) labeling the kinase of interest with a thiol- or amino-reactive fluorophore sensitive to polarity changes in its environment, a thiol-reactive spin label, an isotope or an isotope-enriched thiol- or amino-reactive label such that said fluorophore, spin label, isotope or isotope-enriched label does not inhibit the catalytic activity and/or does not interfere with the stability of the kinase; (d) contacting the kinase obtained in step (c) with a known inhibitor of said kinase; and (e) recording the fluorescence emission signal at one or more wavelengths or a spectrum of said fluorescently labeled kinase of step (c) and (d) upon excitation or (e)′ recording the EPR or NMR spectra of said spin-labeled kinase of step (c) and (d); and (f) comparing the fluorescence emission signal at one or more wavelengths or the spectrum recorded in step (e) or the EPR or NMR spectra recorded in step (e)′; wherein a difference in the fluorescence intensity at least one wavelength, preferably the emission maximum and/or a shift in the fluorescence emission wavelength in the spectra of said fluorescently labeled kinase obtained in step (e), or an alteration in the EPR or NMR spectra of said spin-labeled or isotope-labeled kinase obtained in step (e)′ indicates that the kinase is suitable for the screening for kinase inhibitors.

Adapted to a high-throughput format, multiple kinases or differently labeled variations of the same kinase can be screened.

The term “unsuitable position” in accordance with the present invention denotes a position in the activation loop which was shown to be not suitable for an amino acid labeled according to the invention. This can be due to a decreased sensitivity of the label to changes in its environment or due to predictions based on structural considerations that said position would result in a kinase with a label with decreased sensitivity. The term also encompasses amino acids positioned at a potentially suitable position, wherein a different position is deemed more appropriate. As soon as the number of amino acids having a free thiol or amino group in the activation loop exceeds one, amino acids deemed as unsuitable should be mutated.

Mutating an amino acid includes deleting or replacing said amino acid with another amino acid, provided that said mutation does not result in an inhibited catalytic activity or an interference with the stability of the resulting kinase. Step (b) is carried out if no amino acid having a free thiol or amino group is present in the activation loop of said kinase of interest. The amino acid which is inserted or which replaces another amino acid has to have a free thiol or amino group in order to be labeled.

In a preferred embodiment of the methods of the present invention, the kinase inhibitor binds either exclusively to the allosteric site adjacent to the ATP binding site of the kinase or extends from the allosteric site into the ATP site. These types of inhibitors are also called Type III or Type II inhibitors, respectively. They bind to kinases with higher specificity as compared to Type I inhibitors which bind to the ATP-pocket of the kinase, which is highly conserved in structure among all kinases.

As demonstrated in the examples, the present invention provides means to differentiate between ATP-competitive and non-ATP-competitive inhibitors, enabling for a rapid election of specific inhibitors. The invention is designed to detect the movement of the activation loop of the kinase and is therefore sensitive to all Type II and Type III inhibitors. Although certain Type I inhibitors are either not detected at all or are weakly detected only at high concentrations, some of these inhibitors have induced a robust fluorescence change. Only measurement of the fluorescence change over time (i.e. not an endpoint measurement) can allow Type I inhibitors to be distinguished. As presented in one of the examples below, detected ATP-competitive inhibitors produce an instantaneous fluorescence change (typically <5-10 sec) while Type II and Type III inhibitors bind much slower (seconds to several minutes).

In another preferred embodiment of the kinase or the methods of the present invention, the kinase is labeled at a cysteine naturally present or introduced into the activation loop.

The abundance of cysteines in proteins is usually very low, so that a kinase of the invention can be prepared in a straightforward manner by replacing an amino acid in the activation loop with cysteine and optionally replacing solvent-exposed cysteines with other amino acids. Amino acids containing reactive amines, such as histidine, arginine or lysine or derivatives thereof, are much more abundant and are readily found at the protein surface where they are in contact with the surrounding solvent. Thus, it is preferable to use thiol-reactive labels which can specifically react with an introduced cysteine.

In a more preferred embodiment, the method of screening for kinase inhibitors or the method of generating mutated kinases further comprises step (c1) measuring a fluorescence intensity ratio of two wavelengths recorded in step (c) and obtaining the ratio of the normalized intensity change to the average intensity change (ΔI_{std}). Additionally or alternatively, the maximum standard intensity change (ΔRₘₐₓ) between a kinase labeled according to the invention with inhibitor bound and one without inhibitor may be assessed. A candidate compound is considered a kinase inhibitor or the fluorescent-labeled kinase is considered suitable for the screening for kinase inhibitors if (ΔI_{std}) is >0.25, and/or (ΔRₘₐₓ) is >0.75 and the Z-factor is >0.5. This embodiment relates to the extension of the methods of the present invention to high-throughput scale as described above.

ΔI_{std} is the ratio of normalized intensity change to average intensity of the fluorescence emission. According to de Lorimier et al. (2002), ΔI_{std} is one of the most important criteria for characterizing a fluorescent protein conjugate as suitable for sensitive fluorescence spectroscopy. Ideally, the ΔI_{std} should have a value >0.25 and is calculated by:

$$Δ {I}_{std} = \vert \frac{2 ( {I}_{1} ( {λ}_{std} ) - {I}_{2} ( {λ}_{std} ) )}{{I}_{1} ( {λ}_{std} ) + {I}_{2} ( {λ}_{std} )} \vert$$

where λstd=(λmax, unbound λmax, saturated)/2 and I1, I2 are the fluorescence intensities at std of each spectrum respectively.

ΔRₘₐₓ is the maximum standard intensity change of the fluorescence emission between saturated and unsaturated kinase (REF). According to (de Lorimier et al., 2002), ΔRₘₐₓ is another important criteria for characterizing a fluorescent protein conjugate as suitable for sensitive fluorescence spectroscopy. Ideally, the ΔRₘₐₓ should have a value >1.25 and is calculated by:

$$Δ R = \vert \frac{{}_{}^{O}{A}{}_{1}^{}}{{}_{}^{O}{A}{}_{2}^{}} - \frac{{}_{}^{∞}{A}{}_{1}^{}}{{}_{}^{∞}{A}{}_{2}^{}} \vert$$

where ° A1, ° A2 are the areas in the absence of ligand, and ^{∞}A1, ^{∞}A2 are the areas in the presence of saturating ligand. A computer program can be used to enumerate ΔR for all possible pairs of wavelength bands in the two spectra, to identify the optimal sensing condition, defined as the maximum value of ΔR.

The Z-factor is a statistical measure of the quality or power of a high-throughput screening (HTS) assay. In an HTS campaign, large numbers of single measurements of unknown samples are compared to well established positive and negative control samples to determine which, if any, of the single measurements are significantly different from the negative control. Prior to starting a large screening campaign, much work is done to assess the quality of an assay on a smaller scale, and predict if the assay would be useful in a high-throughput setting. The Z-factor predicts if useful data could be expected if the assay were scaled up to millions of samples. The Z-factor is calculated by:

$$Zfactor = 1 - \frac{3 \times ( {σ}_{p} + {σ}_{n} )}{\vert {μ}_{p} - {μ}_{n} \vert}$$

wherein both the mean (μ) and standard deviation (σ) of both the positive (p) and negative (n) controls (μₚ, σₚ, μₙ, σₙ, respectively) are taken into account.

The measurement of ΔI_{std} and ΔRₘₐₓ as well as the determination of the Z-factor may prove useful in determining whether the label chosen is suitable in the screening for inhibitors. De Lorimier discusses that the measured kinetics and K_{d} obtained with a fluorescent tagged protein will depend on the protein, the ligand and the fluorophore used. Therefore, the same inhibitor binding to the same kinase could give different K_{d} values depending on the label used. The determination of the above values might indicate whether the label chosen is appropriate or whether a different label should be used.

In a further preferred embodiment, the fluorophore or spin-label is not located at or adjacent to phosphorylation sites known or predicted to exist in the labeled kinase. This ensures that the labeling does not interfere with the dynamics of the activation loop or the normal activity and regulation of the kinase which is largely affected by phosphorylation and dephosphorylation.

In another preferred embodiment, said candidate amino acid in the activation loop is identified based on structural and/or sequence data available for said kinase.

For some kinases, structural data, e.g. in the form of crystal or NMR structures is available, wherein the kinase is captured in the activated and/or inactivated state. If such data is available for a kinase, this facilitates the choice of the amino acid position in the activation loop to be replaced for labeling purposes. The actual choice is based on the distance of the position from the allosteric site of the kinase as well as on contacts of the amino acid in said position with other amino acids. If said contacts are deemed essential for the catalytic activity or stability of the kinase, the position is in most cases not suitable for replacement. Additionally, the choice is based on the distance which a particular amino acid will move as the protein changes conformation such that greater distances increase the chance that an environmental change will be detected. However, although distance moved is an indicator of whether a particular position may be useful for labeling, it is the actual change in environment which will correlate directly with the observed changes detected by the attached label.

In a preferred embodiment, the methods of the present invention relating to screening for inhibitors, determining kinetic parameters such as association and dissociation and generating a mutated kinase are combined to obtain a straightforward methodology to obtain specific inhibitors for different kinases. In this regard, any preferred embodiment of a method of the invention may be combined with (preferred) embodiments of other methods of the invention. In a more preferred embodiment of this aspect, an initial screen is carried out using the method of high-throughput screening for kinase inhibitors, followed by a screen using a wide range of concentrations of inhibitors as described above with the method of the invention for determining the kinetics of ligand binding and/or association or dissociation. The latter step is carried out, inter alia, to get an indication of the K_{d} and/or Kₐ value. This step is again repeated by carrying out measurements with a more focused concentration range for more precise measurements of the K_{d} or Kₐ. These measurements may be carried out either as a titration series with the cuvette approach (as described in the examples) and/or real-time kinetic measurements in cuvettes (kₒₙ and k_{off}) to further characterize each inhibitor. Optionally, this sequence of methods is transferred to other kinases or the same kinase labeled differently. This embodiment is designed to enable for high-throughput screening to screen for and characterize a high number of inhibitors in multiple kinases or differently labeled variations of the same kinase.

More specifically, such a combined method is a method for identifying a kinase inhibitor which binds either partially or fully to the allosteric site adjacent to the ATP binding site of a kinase and comprises (a) screening for an inhibitor according to the method of screening for kinase inhibitors of the invention, and (b) determining the rate constant of an inhibitor identified in step (a), wherein a rate constant of <0.140 s⁻¹ determined in step (b) indicates that the kinase inhibitor identified binds either partially or fully to the allosteric site adjacent to the ATP binding site of the kinase. Rate constants of >0.140 s⁻¹ indicate that the kinase inhibitor identified binds in the ATP binding site and does not extend into the adjacent allosteric site. The rate constant is correlated to the reaction time (rate of binding) t_{1/2}: t_{1/2}=ln(2)/k_{obs}. Accordingly, a rate constant (k_{obs}) of <0.140 s⁻¹ corresponds to a reaction time t_{1/2} of >5 s.

The rate constant or rate of binding is preferably determined using the properties of the labeled kinase of the invention. For example, the kinase of the invention can be contacted with an inhibitor and, depending on the label, the fluorescence emission signal of a fluorescently labeled kinase at one or more wavelengths or the electron paramagnetic resonance or nuclear magnetic resonance spectra of a spin-labeled or isotope-labeled kinase can be recorded over time. This corresponds to steps (a) to (c) of the method of determining the kinetics of ligand binding and/or of association or dissociation of a kinase inhibitor of the invention or steps (a) and (b) of the method of determining the dissociation or association of a kinase inhibitor of the invention. In case the rate of binding, i.e. the measurable changes in fluorescence or in the NMR or EPR spectra, is more than 5 seconds after application of the inhibitor, this indicates that the inhibitor is a type II or type III inhibitor.

In another preferred embodiment of the method for screening of kinase inhibitors, the method further comprises (subsequently) optimizing the pharmacological properties of a candidate compound identified as inhibitor of said kinase.

Methods for the optimization of the pharmacological properties of compounds identified in screens, generally referred to as lead compounds, are known in the art and comprise a method of modifying a compound identified as a lead compound to achieve: (a) modified site of action, spectrum of activity, organ specificity, and/or (b) improved potency, and/or (c) decreased toxicity (improved therapeutic index), and/or (d) decreased side effects, and/or (e) modified onset of therapeutic action, duration of effect, and/or (f) modified pharmacokinetic parameters (absorption, distribution, metabolism and excretion), and/or (g) modified physico-chemical parameters (solubility, hygroscopicity, color, taste, odor, stability, state), and/or (h) improved general specificity, organ/tissue specificity, and/or (i) optimized application form and route by a. esterification of carboxyl groups, or b. esterification of hydroxyl groups with carboxylic acids, or c. esterification of hydroxyl groups to, e.g. phosphates, pyrophosphates or sulfates or hemi-succinates, or d. formation of pharmaceutically acceptable salts, or e. formation of pharmaceutically acceptable complexes, or f. synthesis of pharmacologically active polymers, or g. introduction of hydrophilic moieties, or h. introduction/exchange of substituents on aromates or side chains, change of substituent pattern, or i. modification by introduction of isosteric or bioisosteric moieties, or j. synthesis of homologous compounds, or k. introduction of branched side chains, or l. conversion of alkyl substituents to cyclic analogues, or m. derivatization of hydroxyl group to ketales, acetales, or n. N-acetylation to amides, phenylcarbamates, or o. synthesis of Mannich bases, imines, or p. transformation of ketones or aldehydes to Schiff's bases, oximes, acetales, ketales, enolesters, oxazolidines, thiazolidines or combinations thereof.

The various steps recited above are generally known in the art. They include or rely on quantitative structure-action relationship (QSAR) analyses (Kubinyi, “Hausch-Analysis and Related Approaches”, VCH Verlag, Weinheim, 1992), combinatorial biochemistry, classical chemistry and others (see, for example, Holzgrabe and Bechtold, Deutsche Apotheker Zeitung 140(8), 813-823, 2000).

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### Example 1

### Selection of a Suitable Kinase

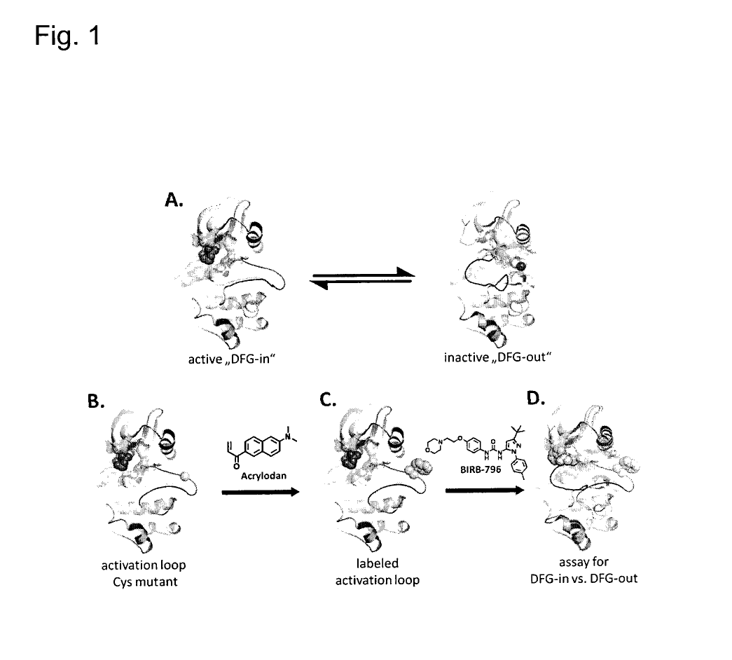

Applicants chose to work with p38α to develop this assay for the following reasons: i) the abundance available of structural information, ii) the availability of crystal structures in both its active and inactive conformations (FIG. 1A.) and iii) the availability of tight binding Type II & III allosteric inhibitors. In the first step, the crystal structures of p38α were closely examined to identify suitable fluorophore attachment sites that would detect allosteric binders. Candidate residues for this mutation must be solvent exposed to enable the attachment of a fluorophore by Michael addition, and exhibit significant movement upon ligand binding. Care was also taken to not choose residues that are critical to maintaining protein stability, catalytic activity or residues in the vicinity of known phosphorylation sites.

A position near the N-terminal end of the activation loop was selected and subsequently mutated into a cysteine residue (FIGS. 1B.,C.). Acrylodan was selected as the fluorophore due to its relatively small size (comparable to a tryptophan side chain), its high sensitivity to polarity changes, its commercial availability and relatively low price. Acrylodan is also known to produce a robust response and should detect movements of the activation loop upon binding of allosteric inhibitors (FIG. 1D.). Before labeling the protein, it was necessary to reduce the chances of fluorophore attachment to any other solvent exposed cysteine residues. Again, structural information was used to locate 4 reduced cysteine residues in p38α. Two of these cysteine are buried within the protein while the other two were solvent-exposed and conservatively mutated into serine. Lastly, a F327L mutation was incorporated to partially activate (Askari et al., 2007: Avitzour et al., 2007) the acrylodan-labeled p38α (ac-p38α) for use in enzyme activity assays, if desired, but is not necessary for functionality of the assay itself.

### Example 2

### Protein Labeling and Fluorescence Characterization

### Protein Labeling

An N-terminal GST-p38α construct containing 4 total mutations (2 cysteine→serine, and the introduction of a cysteine for labeling) was transformed into the BL21(DE3) E. coli strain, overexpressed, purified by affinity, anion exchange and size exclusion chromatography and the pure protein was subsequently used for labeling. Protein and free acrylodan were combined at a 1:1.5 ratio and allowed to react in the dark overnight at 4° C. The conjugated protein (ac-p38α) was concentrated, aliquoted and frozen at −20° C. Mono-labeling of 100% of the protein was verified by ESI-MS. Confirmation of the correctly labeled cysteine is currently being performed by analyzing the tryptic fragments of unlabelled and labeled p38α following a combination of HPLC and ESI-MS or MALDI.

### Fluorescence Characterization

Following labeling, the fluorescent properties of the probe were characterized and initial experiments were carried out using various derivatives of the pyrazolo-urea Type II allosteric inhibitor, BIRB-796 (Pargellis et al., 2002; Dumas et al., 2000 (a and b); Moss et al., 2007; Regan et al., 2002; Regan et al., 2003). The ac-p38α protein labeled on the activation loop shows a strong red-shift from 468 nm to 514 nm with ligand binding (FIG. 2). A large change at 468 nm allows for the possibility of making single-wavelength measurements. However, measuring a ratio of two wavelengths (R=514 nm/468 nm) allows the possibility of eliminating dilution errors between different samples. Using these two wavelengths, the normalized intensity change compared to average intensity (ΔI_{std}) was determined to be 0.50 and the maximum standard intensity change (ΔRₘₐₓ) between saturated and unsaturated ac-p38α was 1.24. These are two of the most important criteria for fluorescence spectroscopy (de Lorimier et al., 2002) and both values together with a Z factor of 0.80 characterize this as a suitable probe for use in fluorescence assays. All further work presented below refers to ac-p38α tagged on the activation loop.

This labeling strategy was also applied to a position on the P-loop of p38α, but the fluorescence response of this probe was not characterized as ideal for use in a screening assay for allosteric inhibitors. However, there is some evidence in the data suggesting that this probe may provide useful information about the equilibrium between the active and inactive states for p38α in the absence of ligand. Additional experiments on this labeled protein are still underway.

### Example 3

### Kinase Expression & Purification

The p38α construct was cloned into a pOPINE vector and was transformed as an N-terminal His-tag construct with Precision Protease cleavage site into BL21(DE3) E. coli. Cultures were grown at 37° C. until an OD600 of 0.6, cooled in 30 min to RT and then induced with 1 mM IPTG for overnight (˜20 hrs) expression at 18° C. while shaking at 160 rpm. Cells were lysed in Buffer A (50 mM Tris pH 8.0, 500 mM NaCl+5% glycerol+25 mM imidazole) and loaded onto a 30 mL Ni-column (self-packed), washed with 3 CV of Ni Buffer A and then eluted with a 0-50% linear gradient using Ni Buffer B (Ni Buffer A+500 mM imidazole) over 2 CV. The protein was cleaved by incubating with PreScission Protease (50 μg/mL final concentration) in a 12-30 mL capacity 10-MWCO dialysis cassette (Thermo Scientific) overnight at 4° C. in Dialysis Buffer (50 mM Tris pH 7.5, 5% glycerol, 150 mM NaCl, 1 mM EDTA, 1 mM DTT). The protein was then centrifuged for 15 min at ˜13,000 rpm to remove any precipitate that may have formed during the cleavage step. The supernatant was then taken and diluted at least 4-fold in Anion Buffer A (50 mM Tris pH 7.4, 5% glycerol, 50 mM NaCl, 1 mM DTT) and loaded onto a 1 mL Sepharose Q FF column (GE Healthcare) and washed with 10 CV of Anion Buffer A. The protein was eluted with a 0-100% linear gradient of Anion Buffer B (Anion Buffer A+600 mM NaCl) over 20 CV. The protein was pooled and concentrated down to 2 mL and passed through a Sephadex HiLoad 26/60 Superdex 75 column equilibrated with Size Exclusion Buffer (20 mM Tris pH 7.4, 5% glycerol, 200 mM NaCl, 1 mM DTT) at a rate of 2 mL/min. The eluted protein was then concentrated to ˜10 mg/mL, aliquoted and frozen at −80° C.

The chicken cSrc gene (residues 251-533; SEQ ID NO: 2) was codon-usage optimized for bacterial expression and synthesized synthetically (Geneart AG, Regensburg, Germany). The chicken cSrc gene was cloned into a pOPINF vector to generate an N-terminal His tag construct containing a PreScission Protease cleavage site. The plasmid was transformed into BL21(DE3) Codon+RIL E. coli for expression. Briefly, cultures shaking at 200 rpm were grown in TB media (containing 1% w/v glucose, chloramphenicol and ampicillin) until reaching an OD₆₀₀˜0.2. The cultures were then cooled to 20° C. for 1 hr prior to induction with 0.3 mM IPTG. The expression continued overnight (approx. 20 hr) at 20° C. The protein was purified using protocols similar to those described previously (Gschwind et al., 2004), with the exception of using PreScission Protease (50 μg/mL final concentration) to cleave the N-terminal His tag. Following size exclusion, the eluted protein was concentrated to ˜10 mg/mL in Size Exclusion Buffer (50 mM Tris pH 8.0, 100 mM NaCl, 5% v/v glycerol, 1 mM DTT), aliquoted and frozen at −80° C.

### Example 4

### Real-Time Measurements

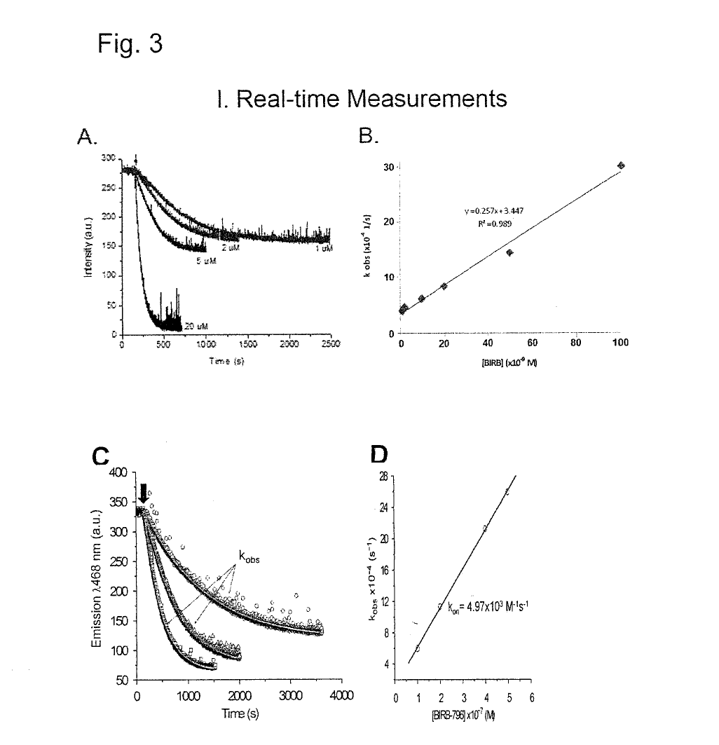

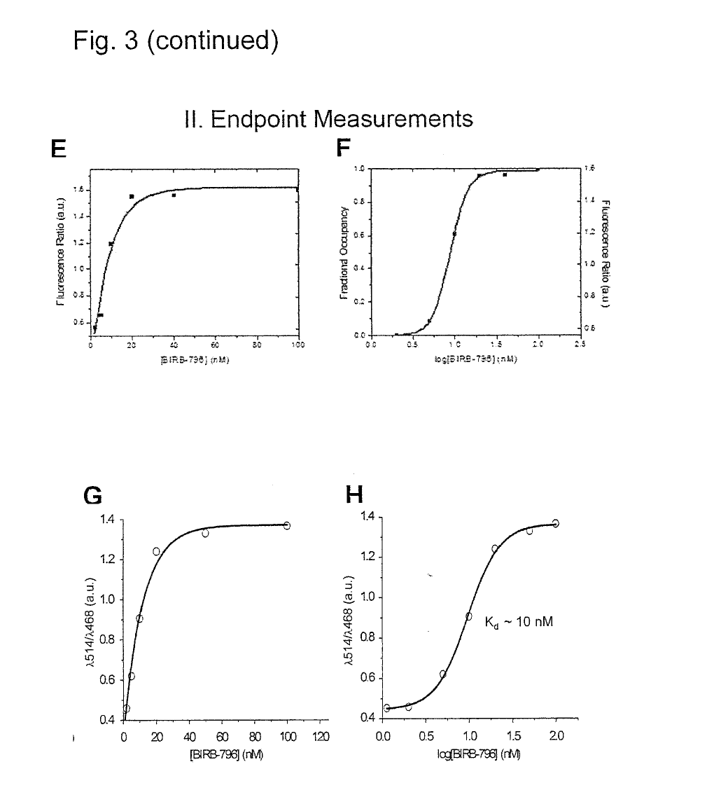

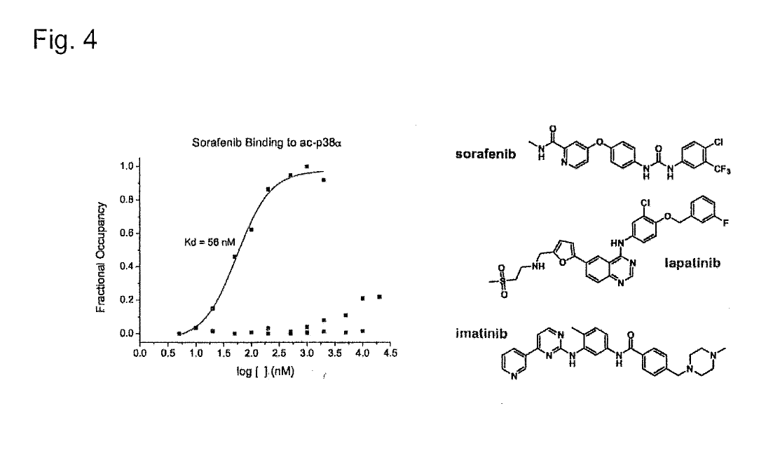

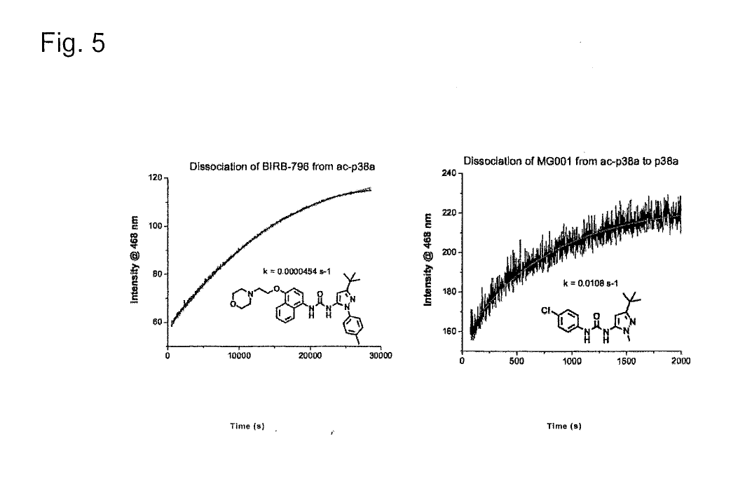

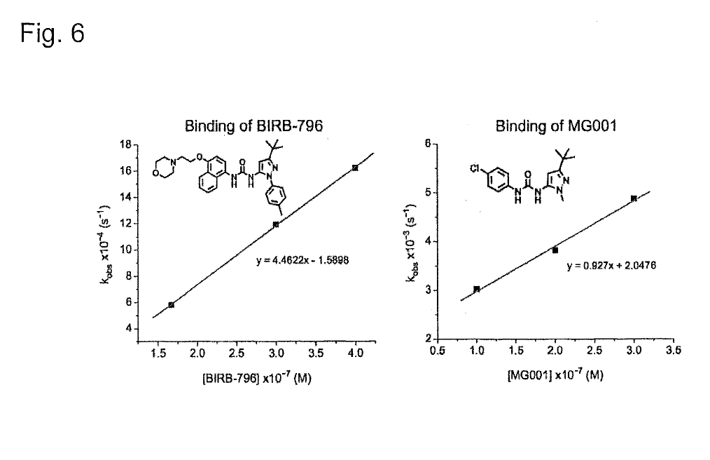

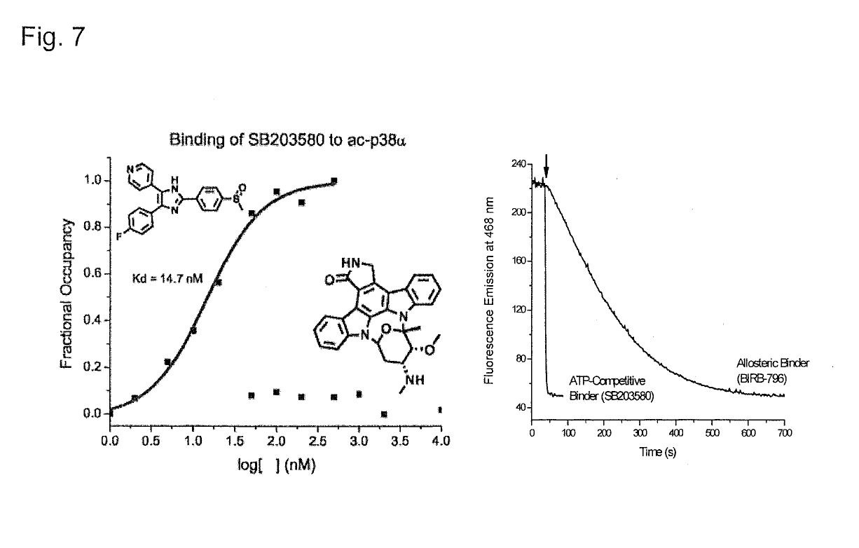

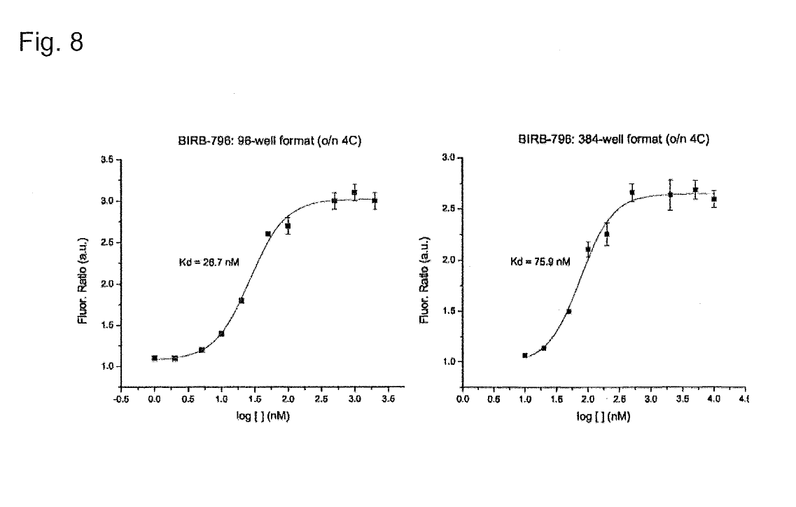

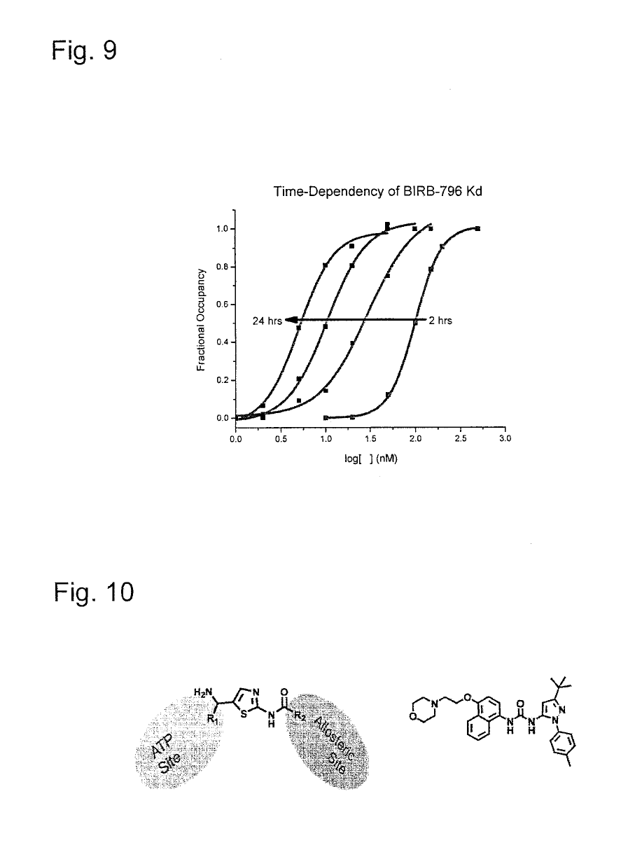

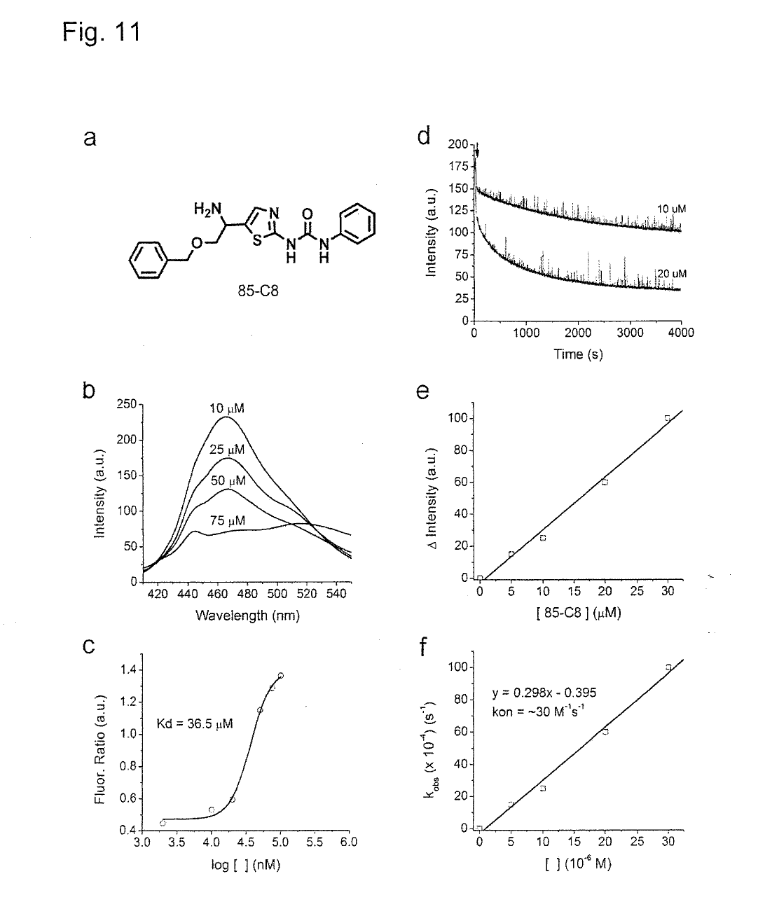

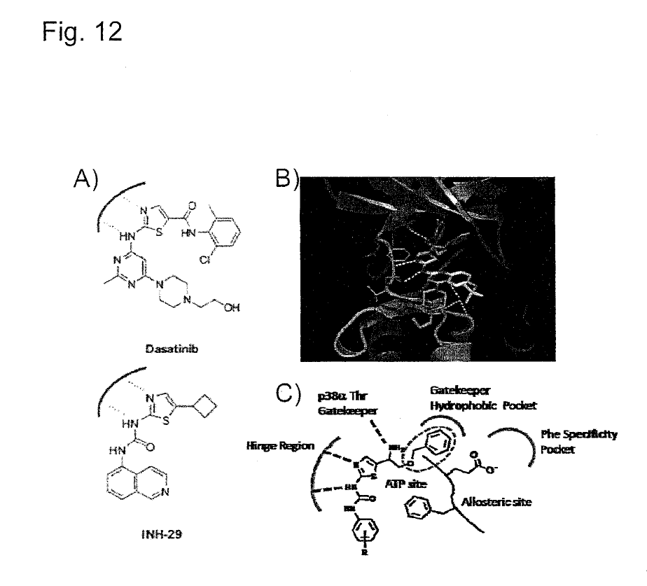

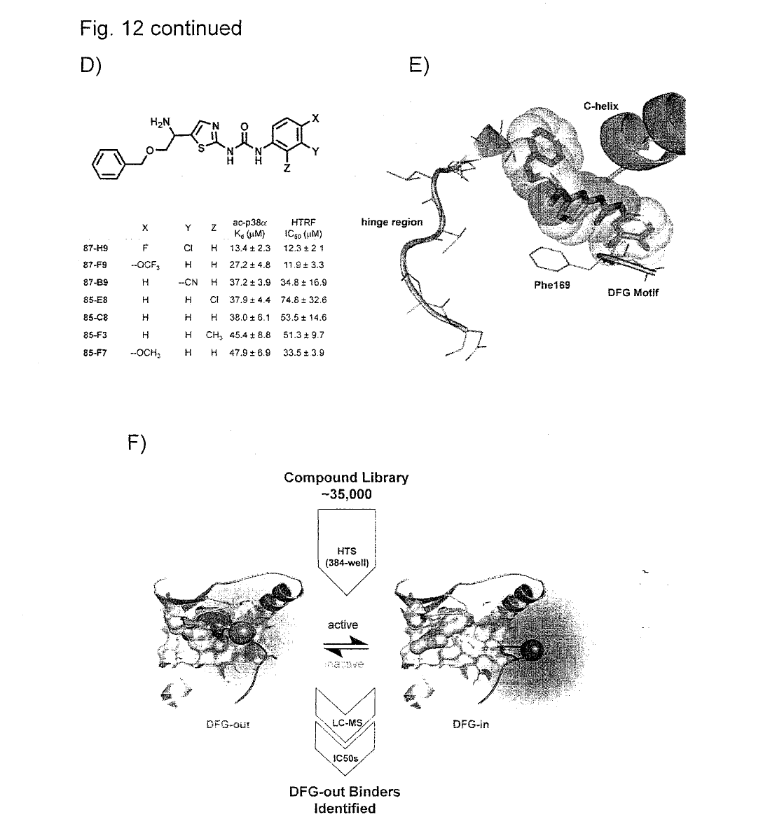

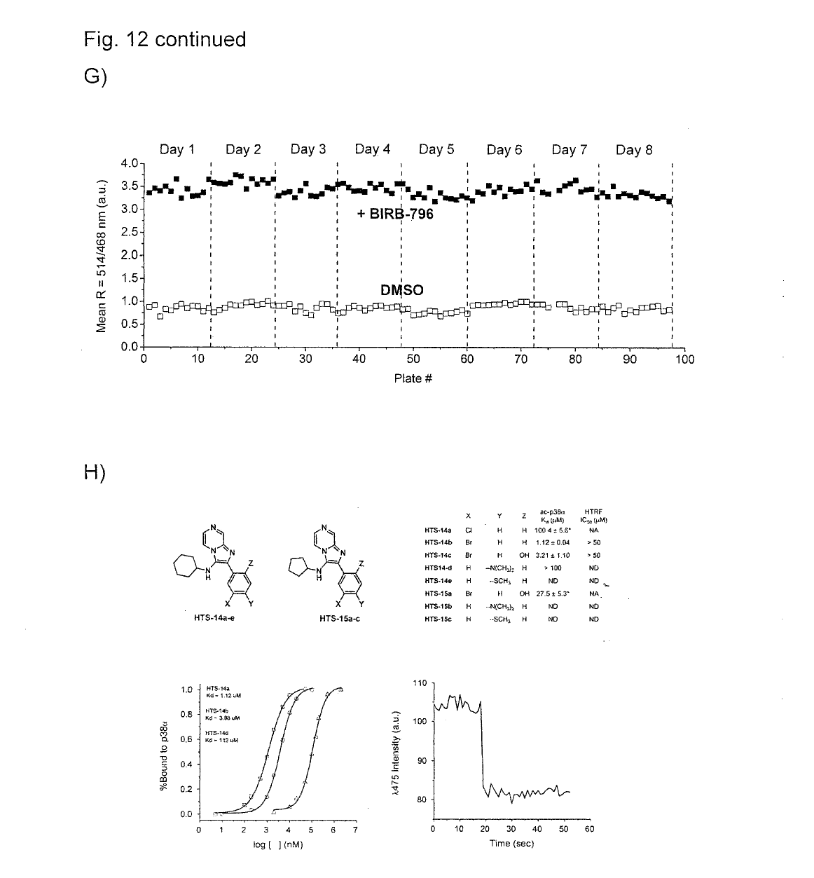

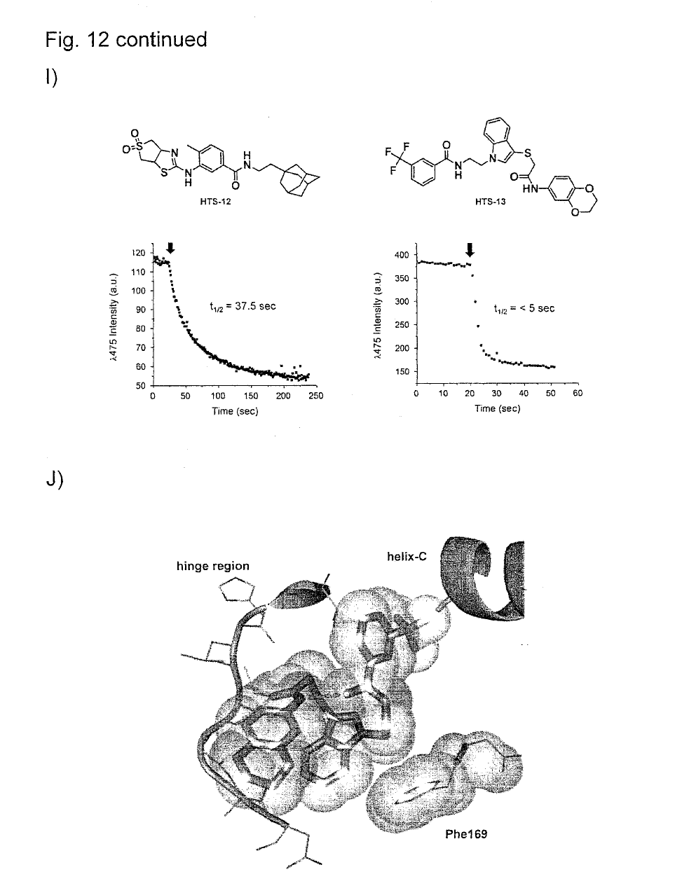

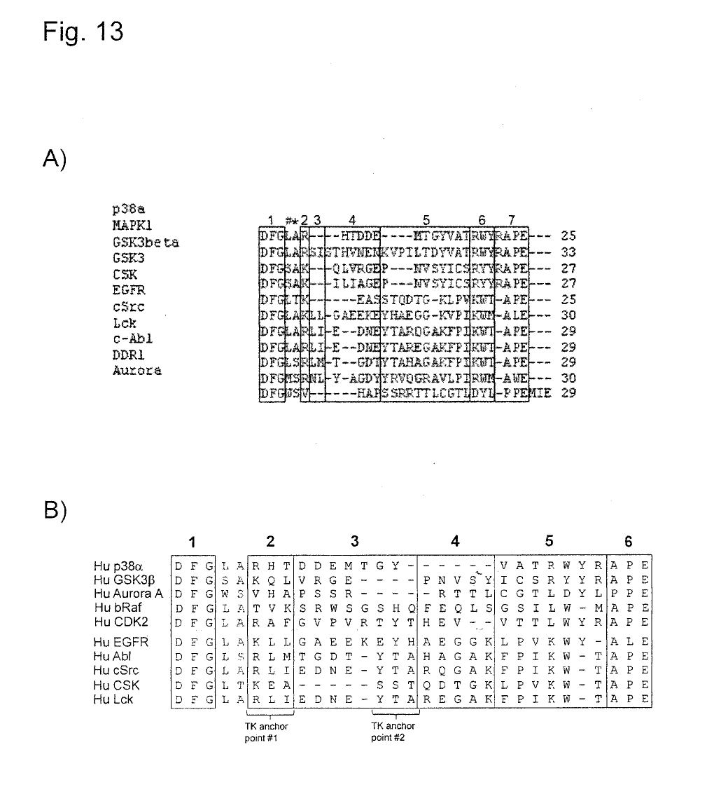

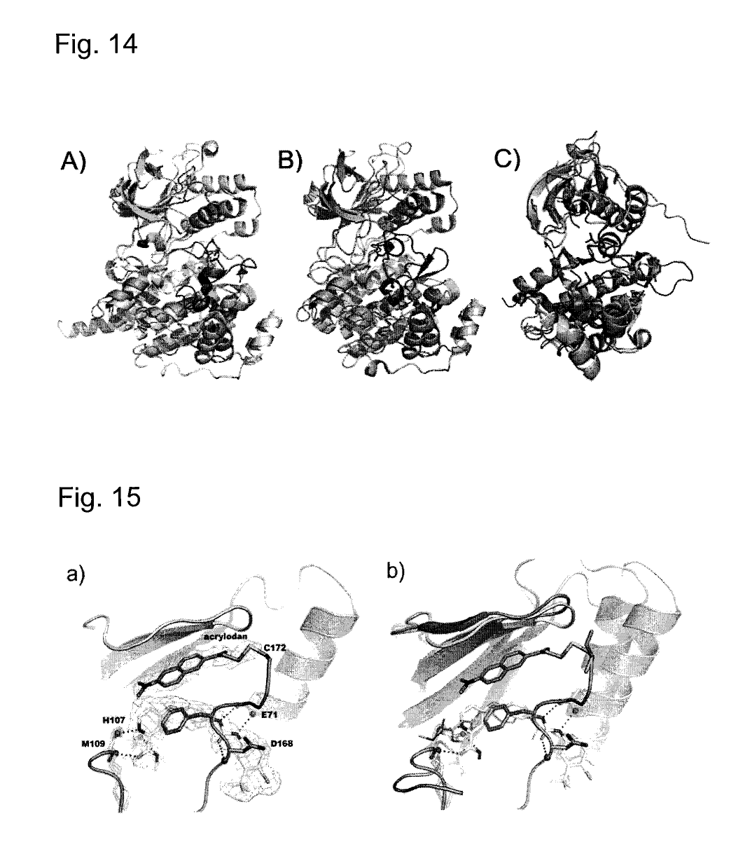

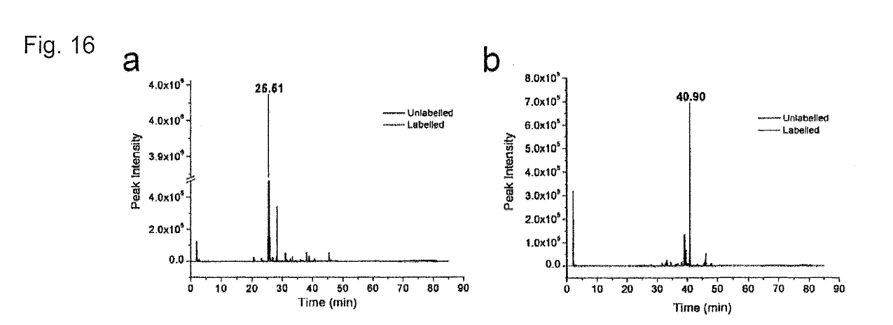

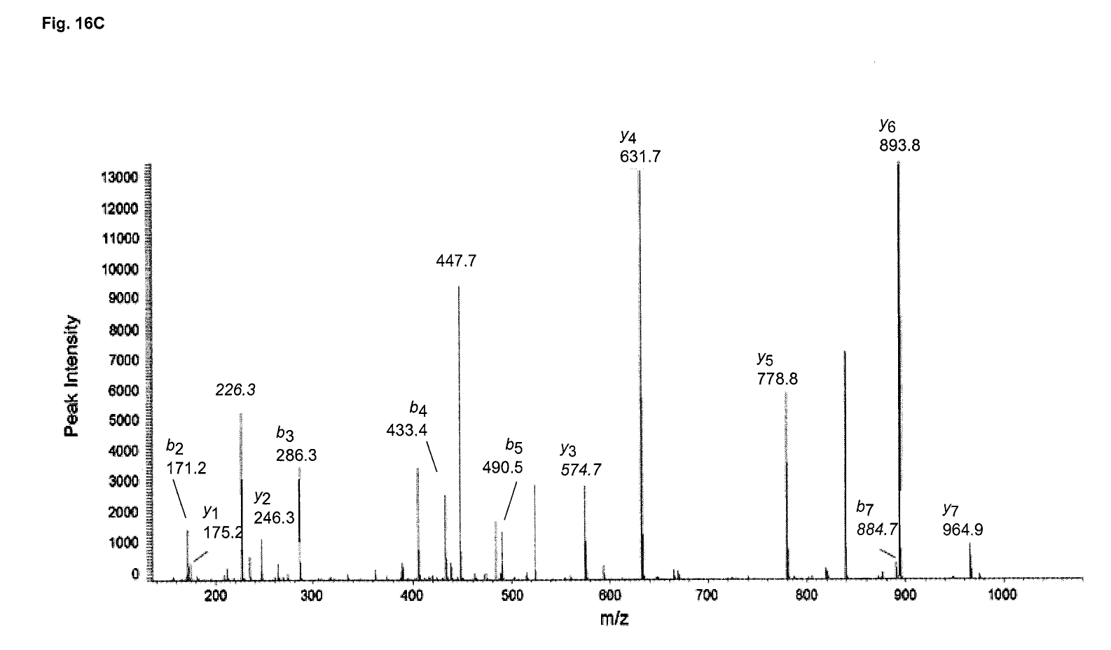

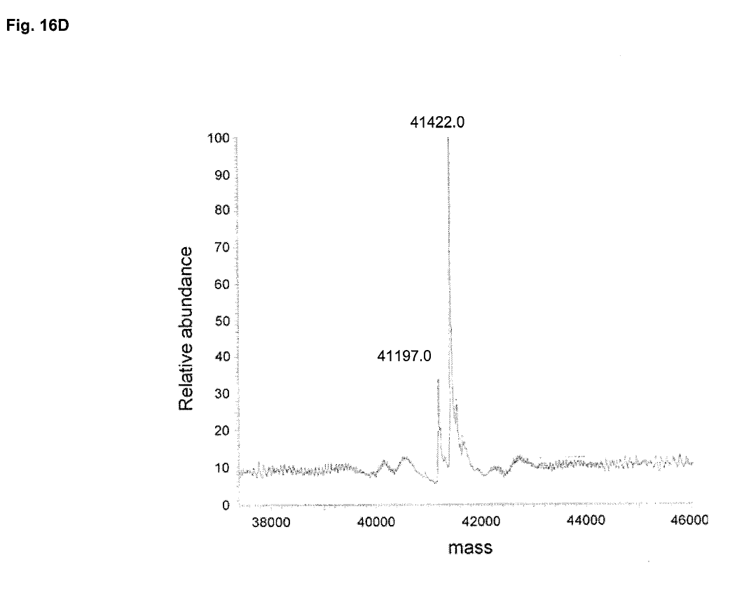

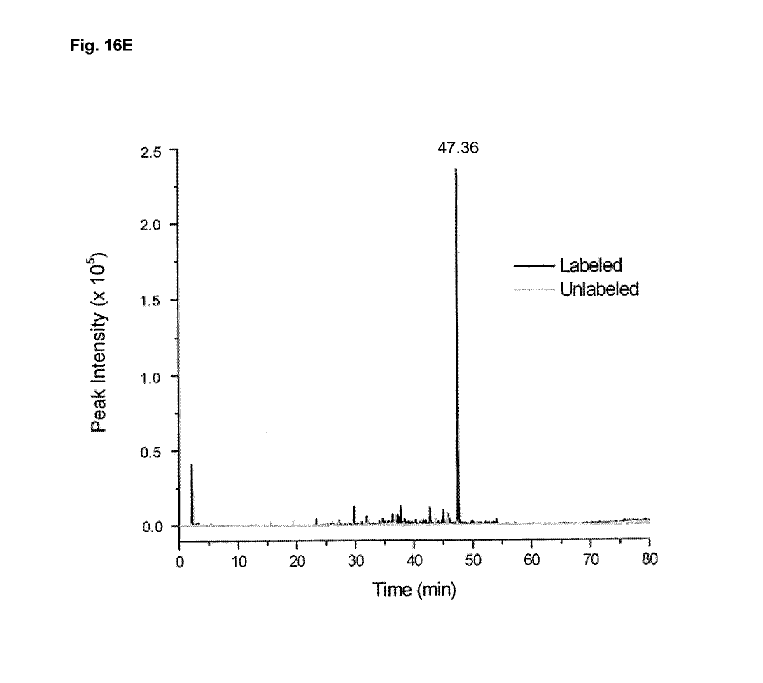

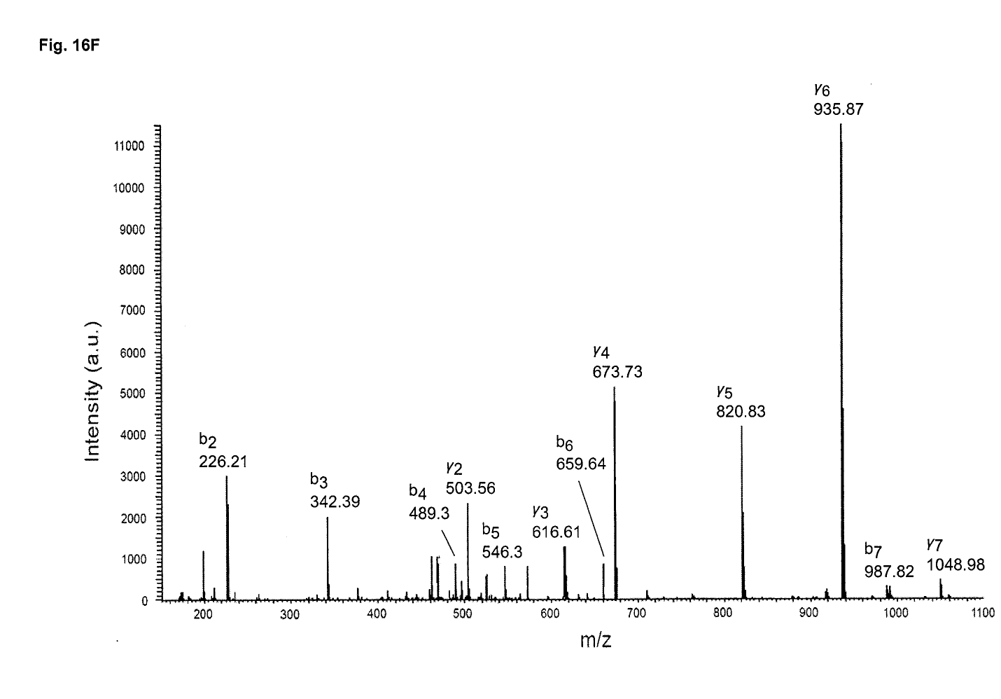

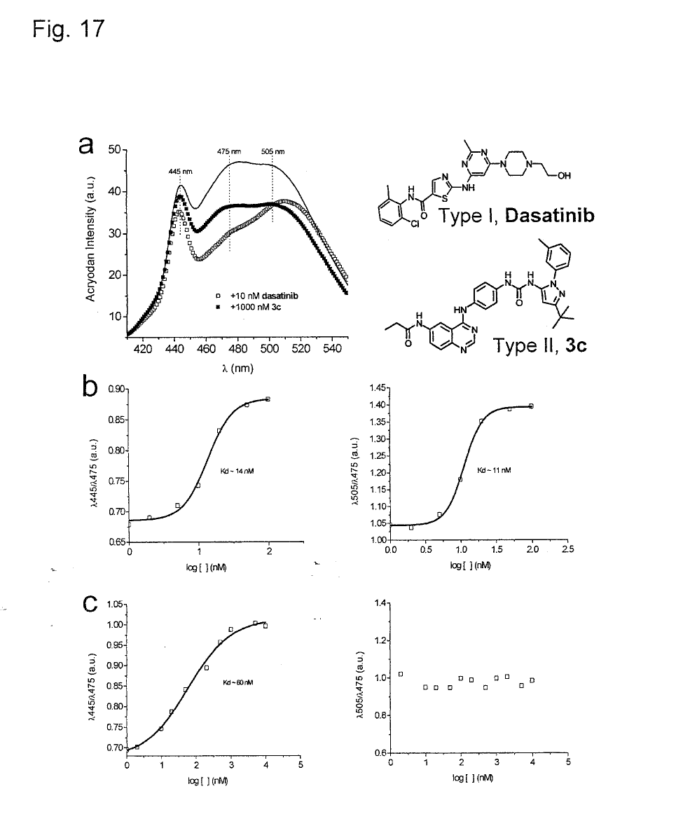

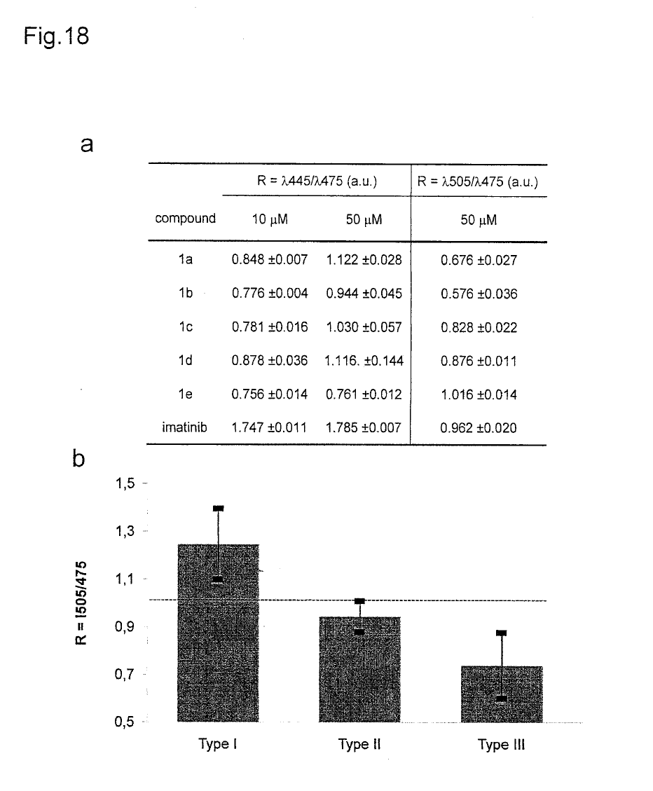

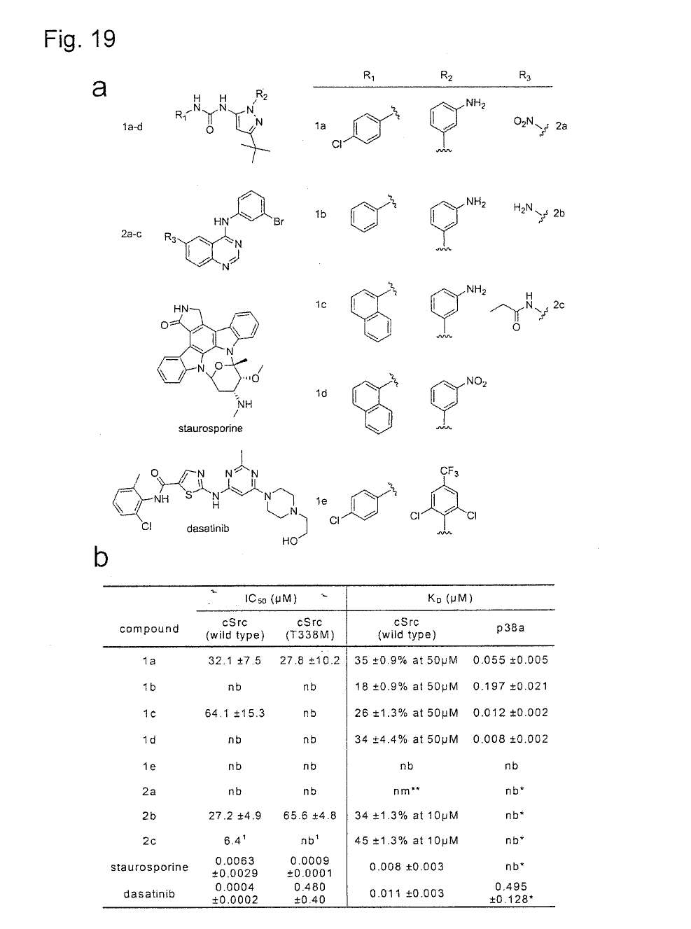

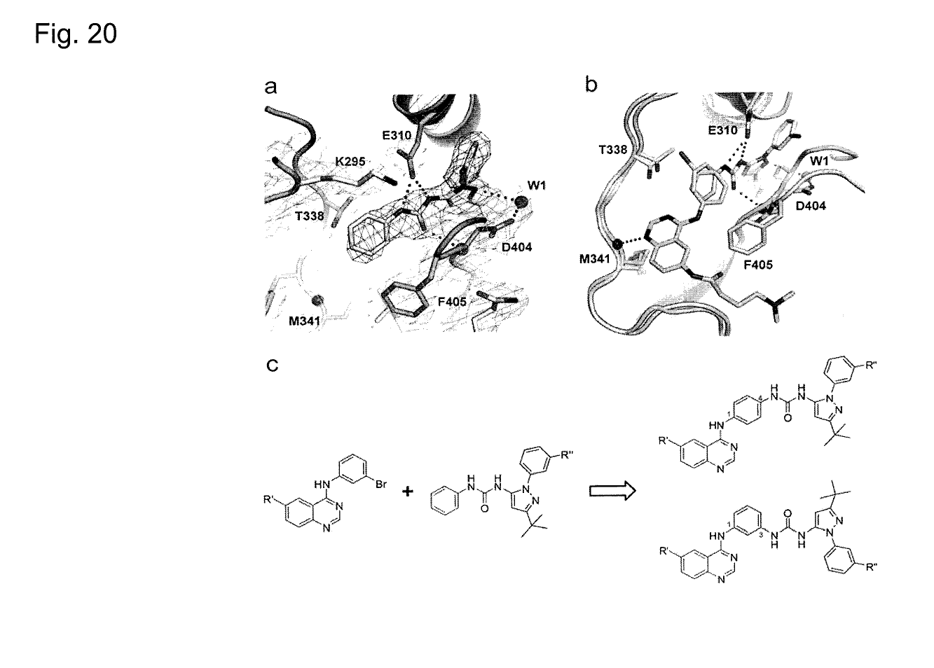

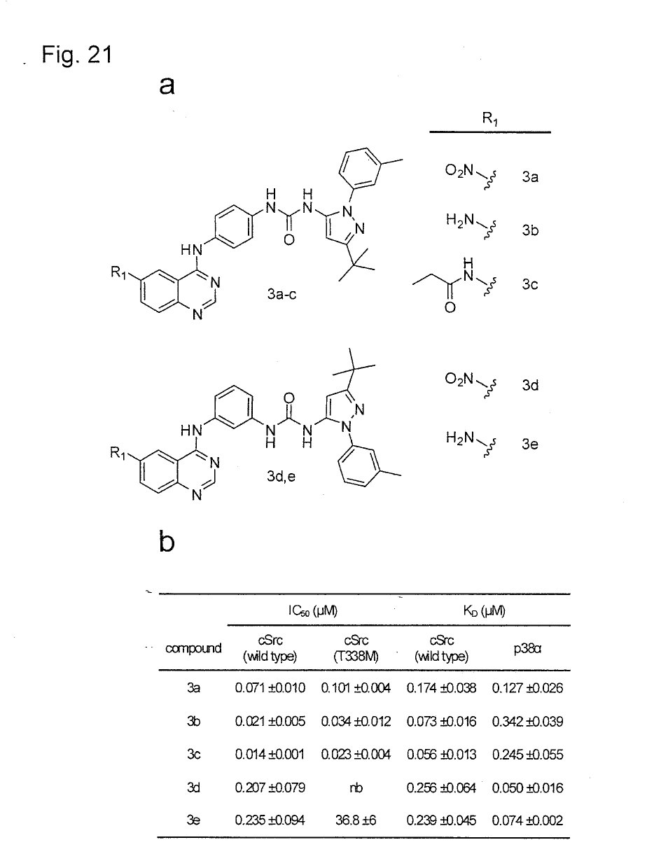

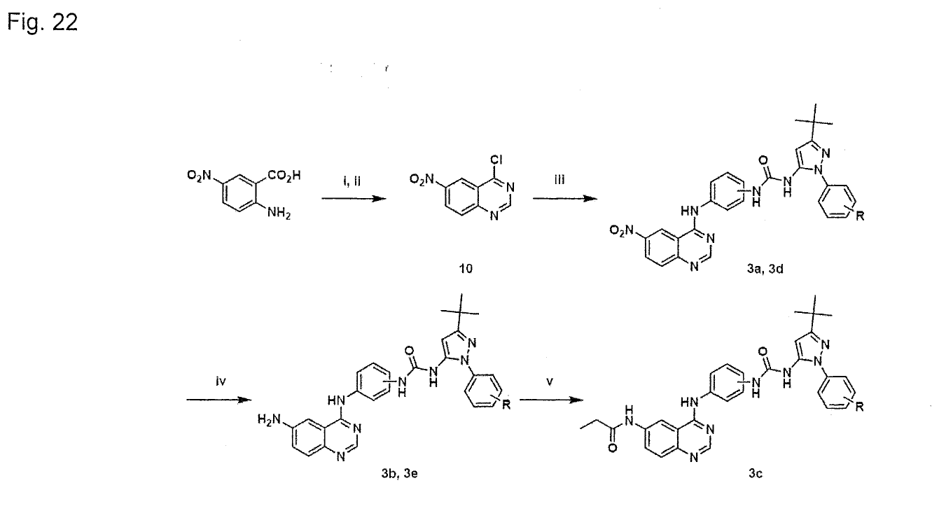

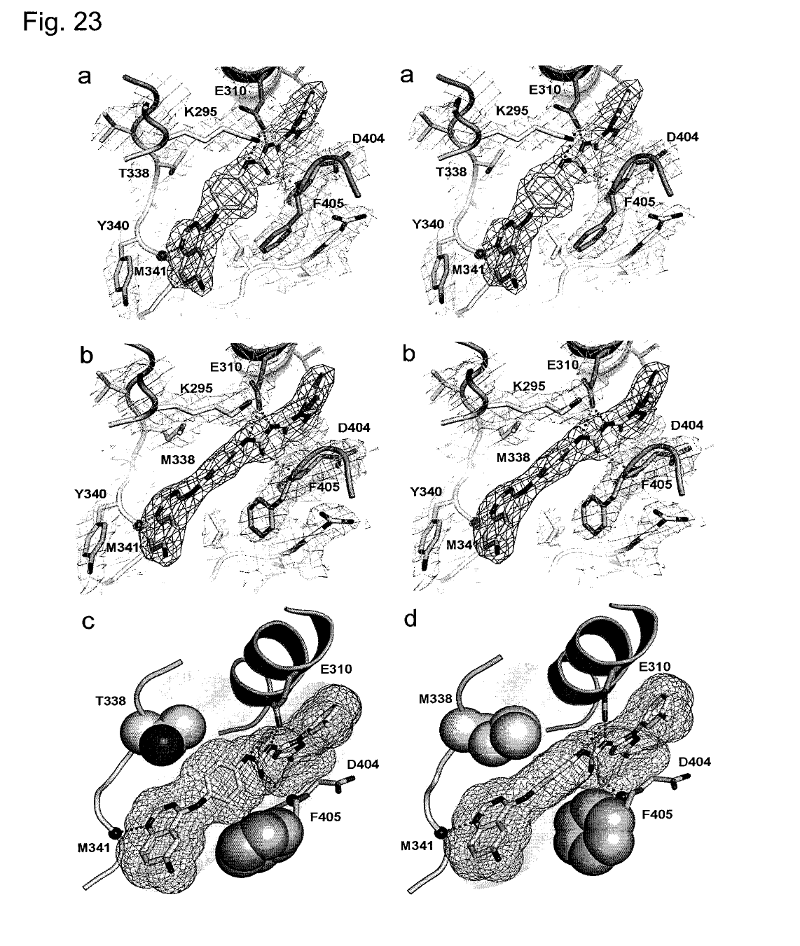

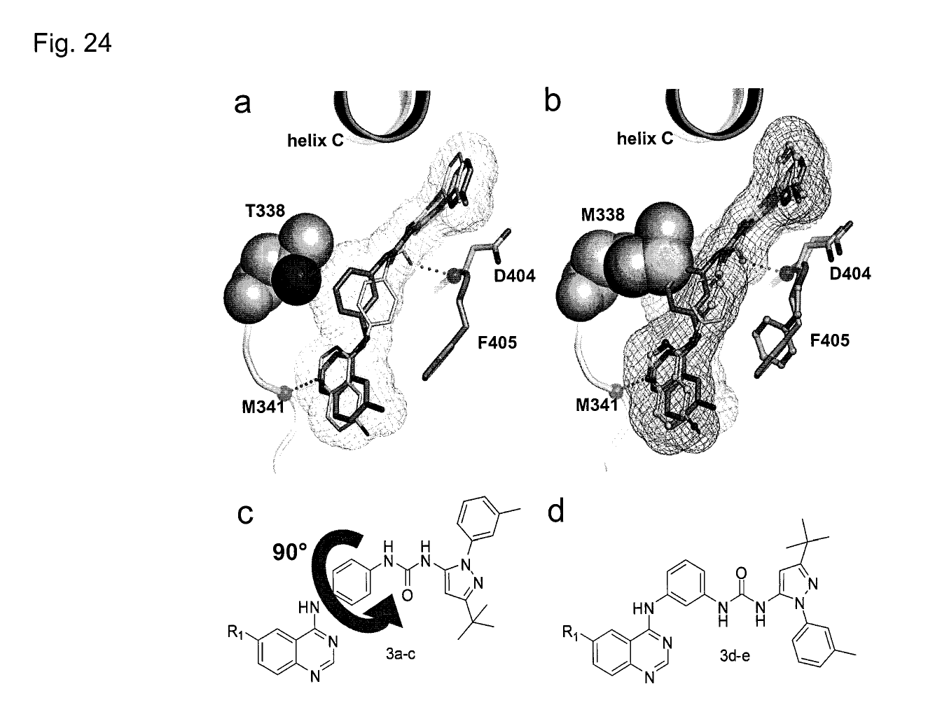

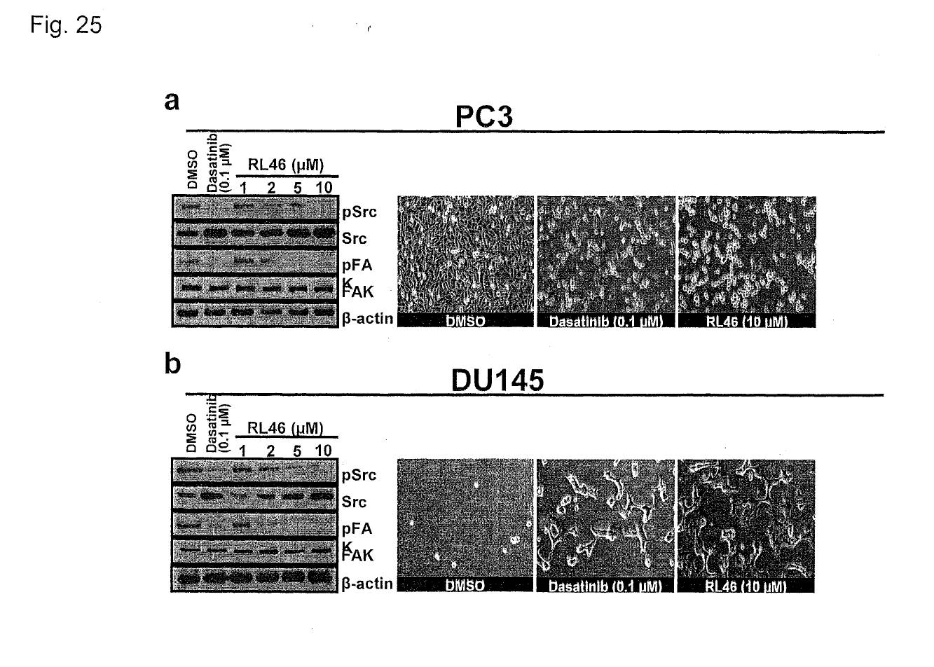

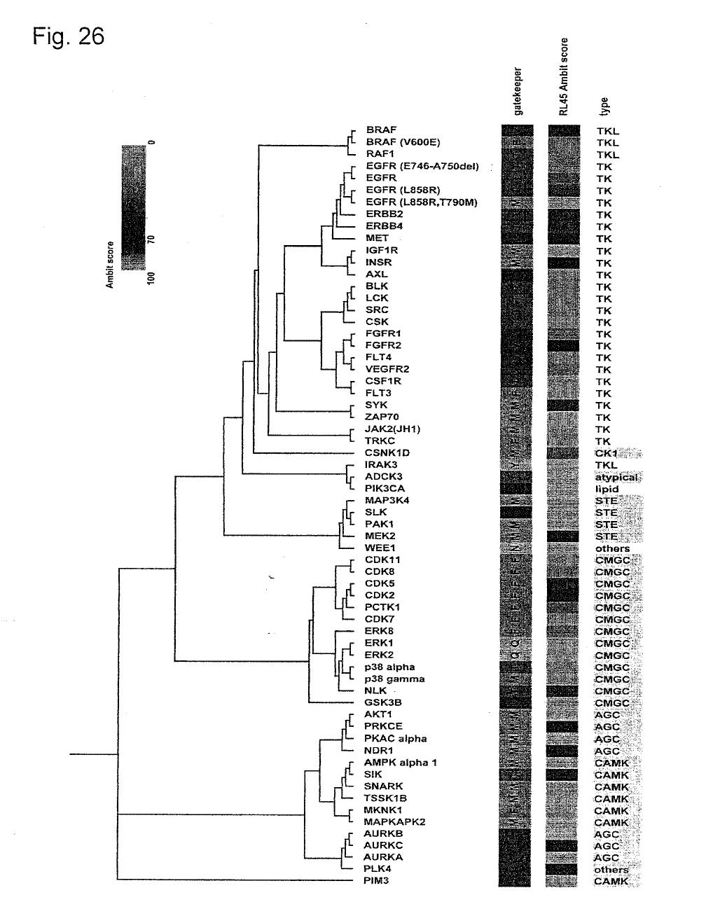

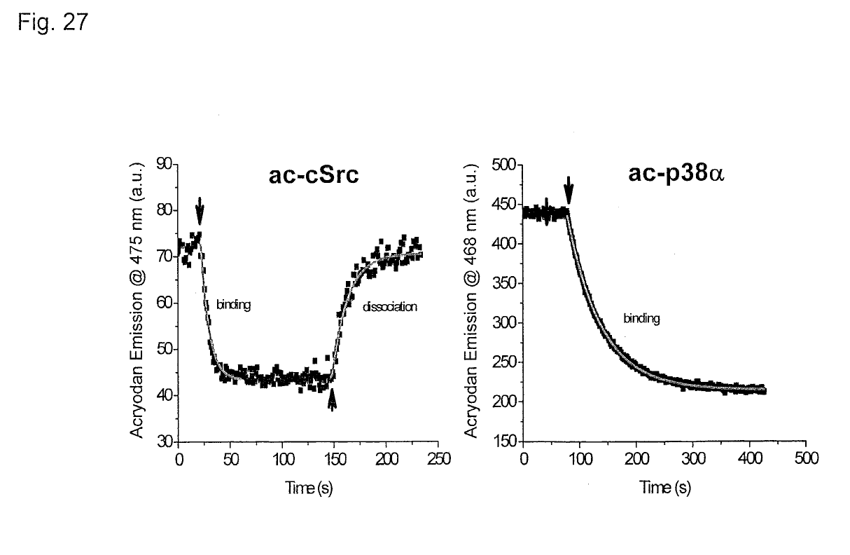

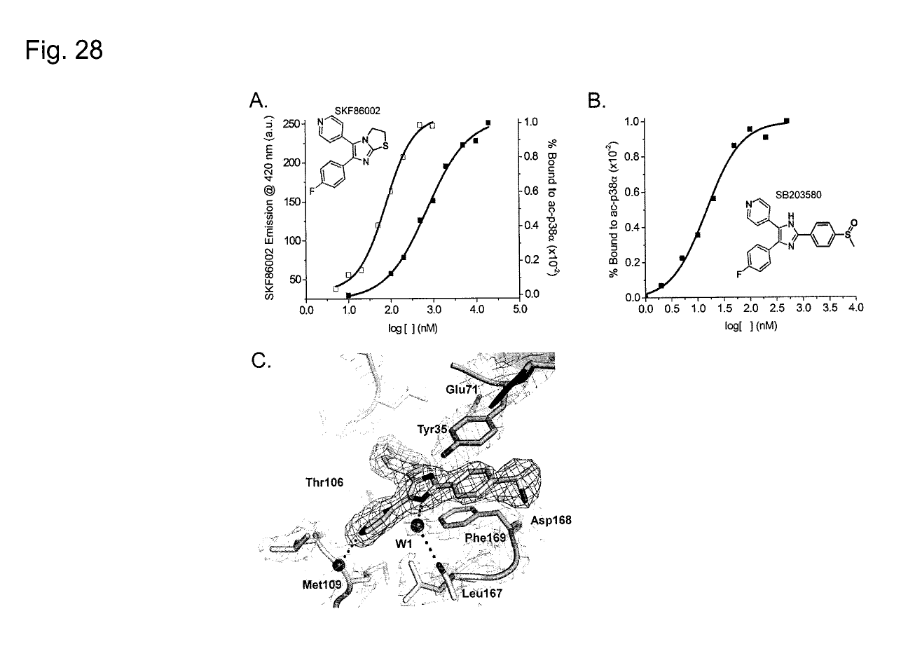

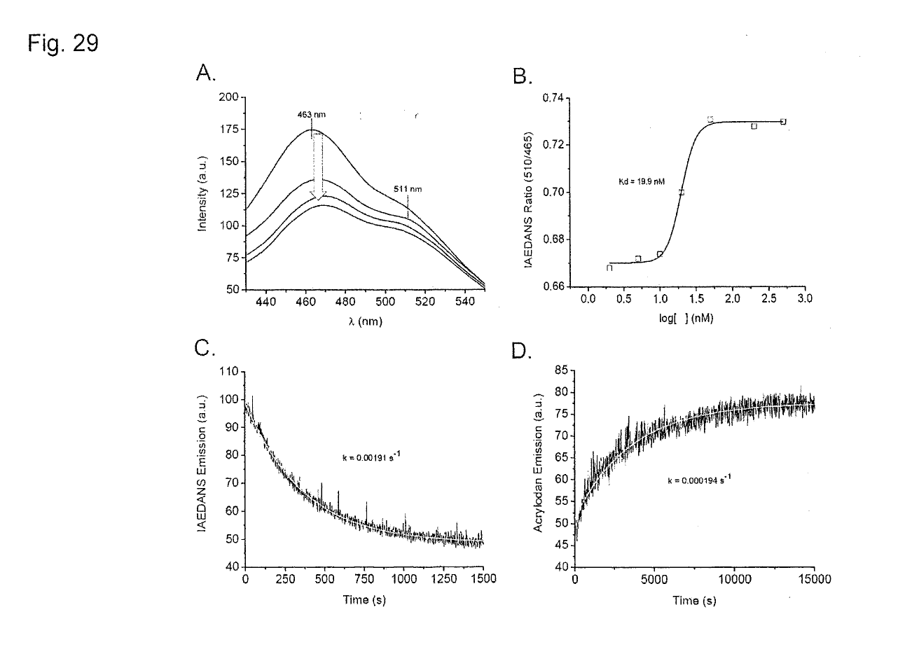

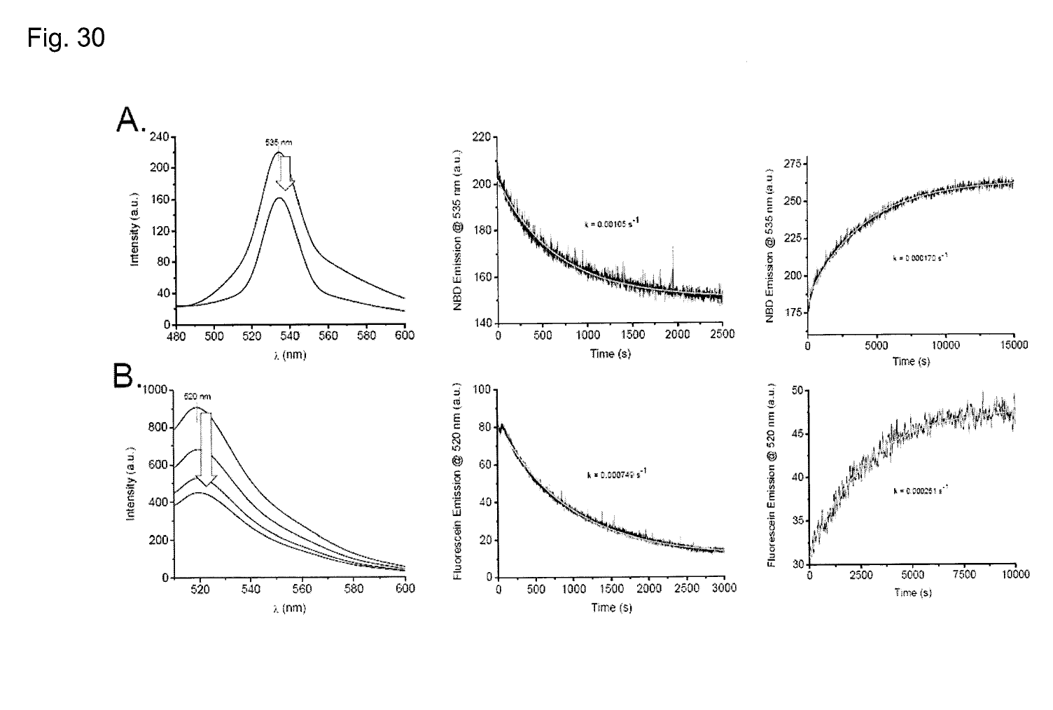

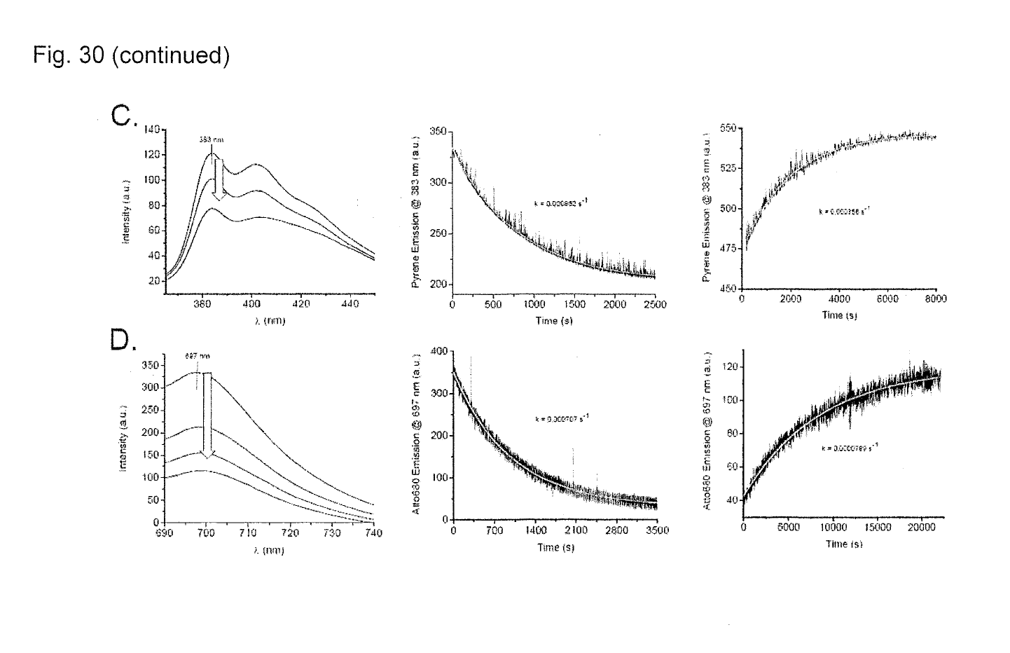

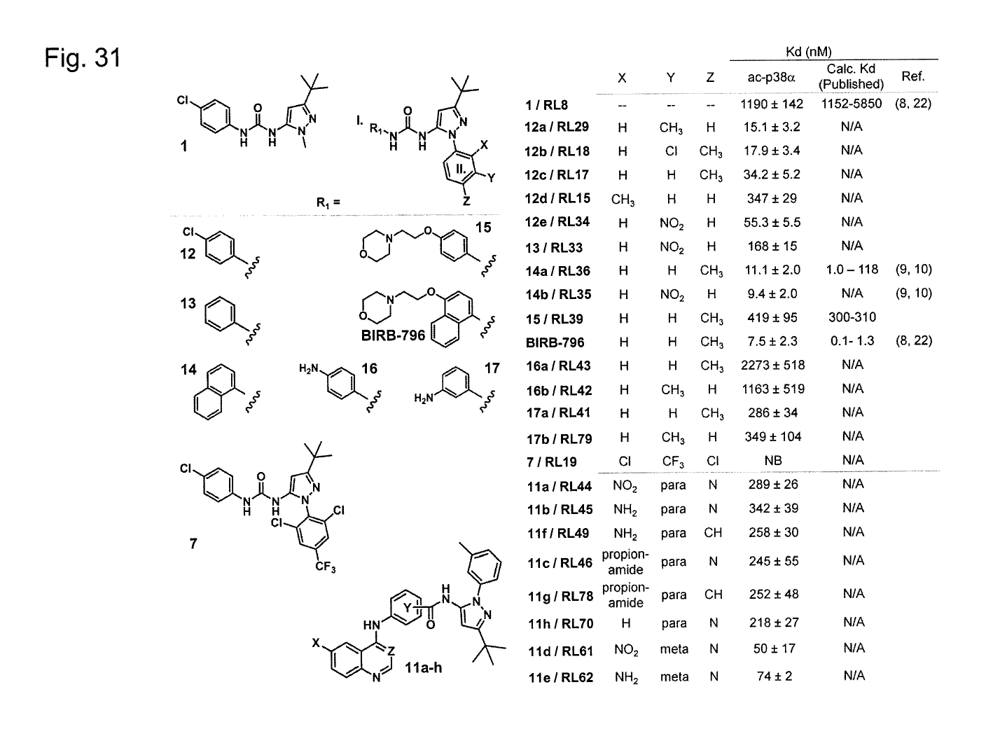

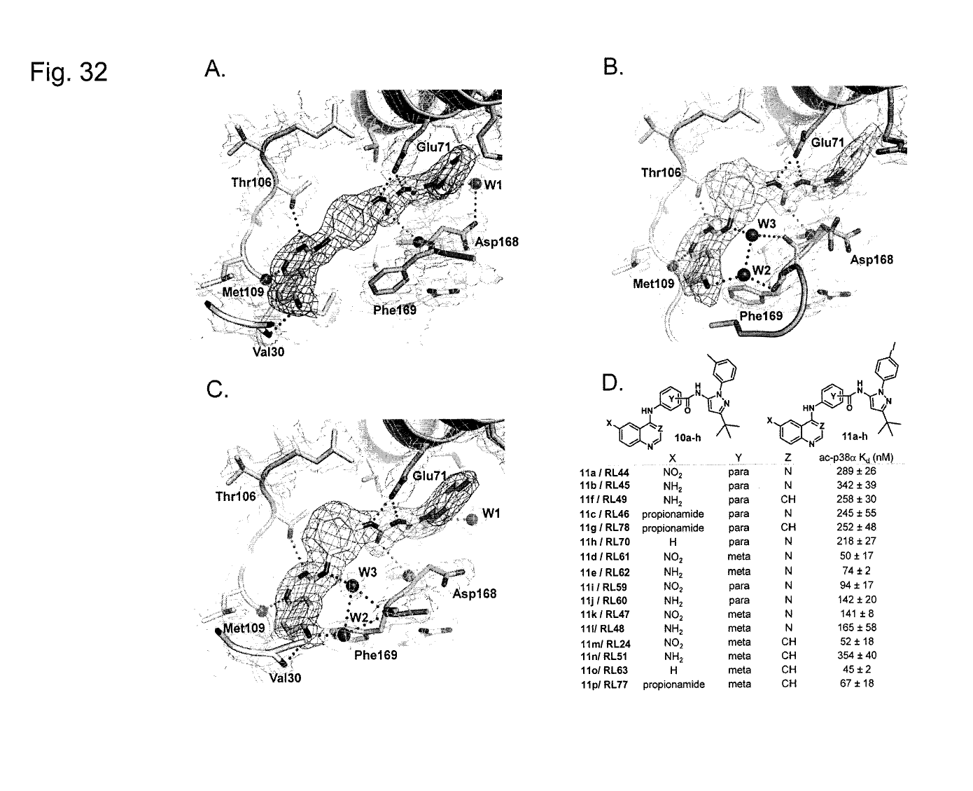

Using polystyrene cuvettes (4 clear sides), real-time measurements of inhibitor binding were performed by delivering various concentrations of BIRB-796 to a suspension of 100 nM ac-p38α. A mini stir bar was placed in the bottom of each cuvette to ensure rapid mixing as inhibitor was delivered through the injection port located above the cuvette. Following addition of the inhibitor, the fluorescence emission at 468 nm decreased in a dose-dependent manner with a first-order kinetic (FIGS. 3A and C). These types of experiments yield rate constants (k_{obs}) which can be plotted and fit linearly to obtain the kₒₙ (slope) and k_{off} (y-intercept) of each compound (FIGS. 3B and D) The kₒₙ for BIRB-796 obtained using this approach (kₒₙ=2.57×10⁴ M⁻¹s⁻¹) is similar to published values (Pargellis et al., 2002; Sullivan et al., 2005), while the estimated k_{off} was 1-2 orders of magnitude faster (k_{off}=3.45×10⁻⁴ s⁻¹) than that measured by other methods (Pargellis et al., 2002; Sullivan et al., 2005). The conditions for such measurements are currently being further optimized (buffer, temperature, protein and inhibitor concentrations, length of incubation).

Such rate measurements were possible with all fluorescent-p38α conjugates tested in this study (see examples 5 and 14). These types of measurements demonstrate the reversibility of the fluorescence response and demonstrate the changing equilibrium which exists between the DFG-in and DFG-out conformations.

Current attempts at directly measuring k_{off} by adding an excess of unlabelled protein to a suspension of ac-p38α bound with inhibitor are currently underway (see example 7). Several additional kₒₙ measurements were also performed using a new preparation of ac-p38α and inhibitor.

### Example 5

### Endpoint Measurements

Before scaling to a 384-well plate format, initial Kd measurements were carried out in cuvettes until conditions could be optimized (buffer, temperature, protein and inhibitor concentrations, length of incubation). Simple binding equilibrium experiments were carried out to determine the Kd of BIRB-796 binding to p38α. Individual cuvettes containing 50 nM ac-p38α and various concentrations of BIRB-796 (1-100 nM) were incubated at 4° C. overnight and measured 24, 48, 72 and 96 h later. Applicants found that the Kd of BIRB-796 was time-dependent, as reported elsewhere (Pargellis et al., 2002), which necessitates longer incubation times for Type II inhibitors. All Type III inhibitors required only an overnight incubation.

TABLE 1
Measured K_{d} values of pyrazolourea derivatives of BIRB-796

| Compound | Kd (nM) | designation |
|---|---|---|
| 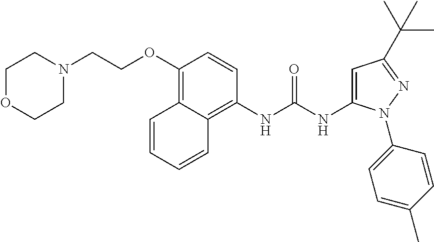 | 5 | BIRB-796 |
| 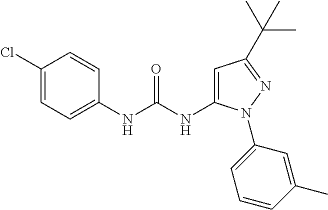 | 15 | 12a, RL29 |
| 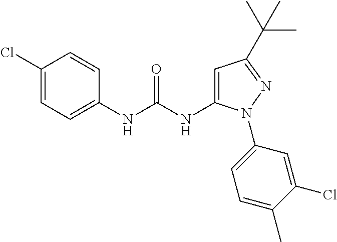 | 18 | 12b, RL18 |
| 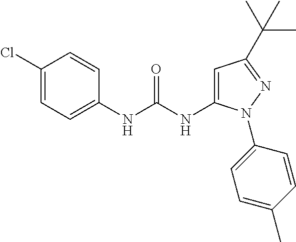 | 34 | 12c, RL17 |
| 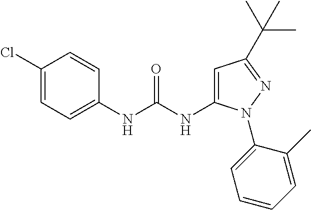 | 347 | 12D, RL15 |
| 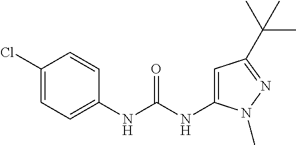 | 1.190 | 1, RL8 |
| 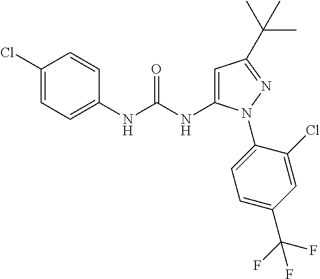 | No Binding | 7, RL19 |
| 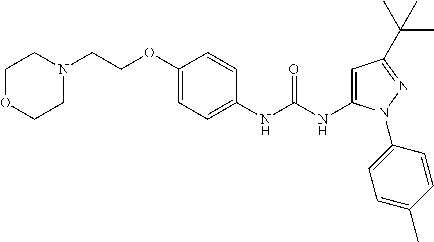 | 419 | 15, RL39 |
| 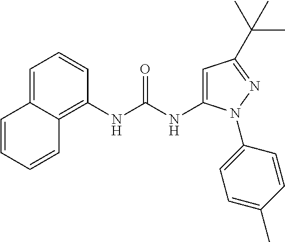 | 11 | 14a, RL36 |
| 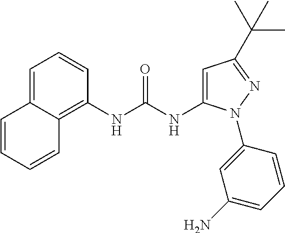 | 12 | 5, RL38 |
| 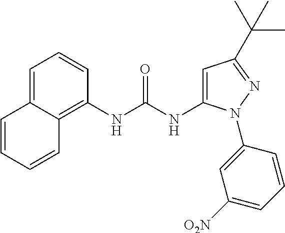 | 3 | 6, RL35 |
| 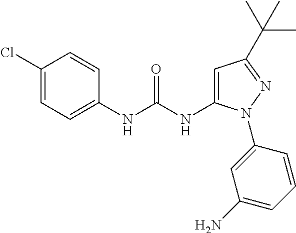 | 55 | 3, RL57 |
| 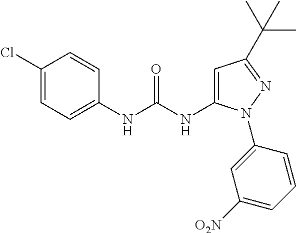 | 19 | 12e, RL34 |
| 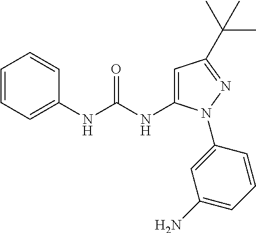 | 197 | 4, RL37 |
| 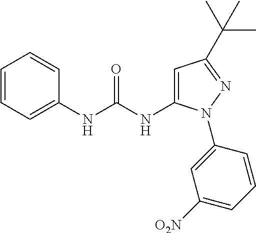 | 162 | 13, RL33 |

The emission spectrum of each sample was measured and the fluorescence ratio (R) was calculated and plotted to show the saturation of ac-p38α in the inactive state (FIGS. 3E. and G.) or plotted on a logarithmic scale to determine the Kd (FIGS. 3F. and H.). Similar experiments were carried out for a focused pyrazolo-urea library of 15 compounds synthesized in the group with varying affinities for the allosteric site of p38α. The compounds and their Kd values are listed in Table 1. Kd values determined using this probe vary as much as 10-fold from published values (Pargellis et al., 2002; Dumas et al., 2000 (a and b); Moss et al., 2007; Regan et al., 2002; Regan et al., 2003; Sullivan et al., 2005) with the largest differences occurring for compounds with a published Kd of <10 nM. However the Kd values follow the same trend as found in the literature. Although lowering the concentration of ac-p38α in the assay would likely improve the values obtained for the tightest binding compounds, a concentration of 50 nM probe has been determined to be the lower limit that can be used to obtain reproducible data with high signal-to-noise. It is also worthy to note that all published Kd values for these compounds are calculated from rate constants (k_{off}/kₒₙ) and not measured directly.

### Example 6

### Extension of Endpoint Measurements to Further Compounds

Several additional Type II inhibitors were tested using endpoint measurements to obtain the Kd of binding to p38α. The most important feature of these compounds is that they do not share the pyrazolourea scaffold of Applicants' numerous other compounds which were used to initially characterize the assay. This was a crucial step towards demonstrating that the change in fluorescence is dependent only on the change in protein conformation and not on the drug scaffold which is bound.

Of particular importance are the results obtained for the drugs lapatinib (Tykerb) and imatinib (Gleevec), selective potent Type II inhibitors of EGFR and Abl/PDGFR kinases, respectively. Addition of these compounds to ac-p38α did not result in a fluorescence change or measurable Kd for either compound. However, addition of Sorafenib (Nexavar), a well-known bRaf and VEGFR2 inhibitor, produced a strong fluorescence response indicative of allosteric binding to p38α. The data obtained for these compounds is shown in FIG. 4.

In a recent publication by scientists at Ambit Biosciences, 38 known kinase inhibitors were screened against a panel of 317 kinases and Kd values were measured in an attempt to quantitate inhibitor binding to off-target kinases (Karaman et al., 2008). They found that lapatinib and imatinib do not bind to p38α, while sorafenib binds with a Kd ˜370 nM.

Sorafenib was the first allosteric compound of another drug scaffold to validate this assay. The Kd of sorafenib was found to be time-dependent, similar to other Type II inhibitors, resulting in Kd values of 115 nM and 56 nM after 6 and 24 hr incubation times, respectively. These values are similar to the published Kds for sorafenib against its intended kinase targets, bRaf and VEGFR2. The higher Kd value obtained in the Ambit study for binding to p38α is likely the result of the standard conditions of their screen in which inhibitors and protein were only incubated for 1 hr.

Validation of Sorafenib as a type II p38 Inhibitor

To confirm that Applicants' new assay approach was correctly reporting the binding of sorafenib to the DFG-out conformation of p38α, Applicants co-crystallized it with wild type p38α and solved the structure to a resolution of 2.1 Å. Applicants found that sorafenib adopts a Type II binding mode with the activation loop of p38α in the DFG-out conformation. The halogenated phenyl moiety of sorafenib resides in the allosteric site and Glu71 of helix C forms a pair of symmetric hydrogen bonds to both urea nitrogens. The N-methyl-carboxamide of the inhibitor hydrogen bonds (2.7 Å) with the backbone NH of Met109 (hinge region) and the phenoxy oxygen approaches the O^{γ} of Thr106 (3.6 Å) (gatekeeper residue) and coordinates a water molecule (3.4 Å) that can also hydrogen bond with the backbone carbonyls of Leu104 (3.3 Å) and Ala51 (2.8 Å) and O^{γ} of Thr106 (3.3 Å). The interaction of sorafenib with the gatekeeper via a water-mediated hydrogen bond has not been reported elsewhere, thereby allowing for the possibility for further inhibitor optimization. Structural alignment of sorafenib complexed to p38α and b-Raf reveal that the inhibitor is pulled more towards the hinge region in b-Raf to form two hydrogen bonds with the back bone of Cys531 (Met109 in p38α). In the p38α complex, the hinge region of the kinase adopts an extended conformation and the N-methylcarboxamide-substituted pyridine ring of sorafenib rotates 180° around its phenoxy moiety and now points towards the N-lobe of the kinase and away from the hinge region. This movement positions the pyridine ring close to the side chain of Phe169 of the DFG-motif and allows for electrostatic interactions (edge-to-face orientation of both π-electron systems), suggesting an additional stabilizing role for this interaction. This cross-talk between several Type II inhibitors in complex with p38α presented here may provide further opportunities for the development of inhibitors that not only induce the inactive kinase conformation but also stabilize it by interacting with Phe169 directly within the ATP binding site.

### Example 7

### Reversibility of Fluorescence—Effect of ATP & Inhibitor Dissociation

Since the DFG-in and DFG-out conformations of kinases are believed to be a dynamic equilibrium, it was important to demonstrate the reversibility of the fluorescent change observed in the presence of allosteric binders. Applicants have obtained titration curves for 1, RL8 in the presence and absence of intracellular concentrations of ATP (5 mM). 1, RL8 was chosen since it is the weakest allosteric binder in Applicants' compound collection and likely to be competed out of the kinase by high concentrations of ATP, which would shift the kinase more towards the DFG-in conformation. As expected, the binding curve of 1, RL8 was significantly affected by the presence of ATP, resulting in a higher measured Kd of 1.62 μM.

Another set of measurements was then attempted to demonstrate the reversibility of the fluorescence change by inducing inhibitor dissociation. After allosteric binders were added to and allowed to equilibrate with ac-p38α, a 10-fold excess of non-labeled p38α was added to the cuvette while monitoring the fluorescence of acrylodan at 468 nm. The addition of excess kinase causes the inhibitor to redistribute, resulting in a net dissociation of inhibitor from ac-p38α and a fluorescence increase which was fit to a first-order function. A 10-fold excess of unlabeled kinase is a standard protocol used to ensure that the rate of dissociation would reflect the true k_{off} from the protein (Hibbs et al., 2004). Adding smaller amounts of unlabelled kinase would likely not force the dissociation of inhibitor as effectively, resulting in artificially slower dissociation rates. Normally, addition of an allosteric inhibitor results in a fluorescence decrease in the case of ac-p38α. Measurement of the dissociation of BIRB-796 and 1, RL8 are shown in FIG. 5 (RL8 is called MG001 in the figure) and FIGS. 29 and 30 for BIRB-796.

These measured k_{off} values are different from those published by Pargellis et al. by a factor of 10 for both BIRB-796 and 1, RL8 (Pargellis et al., 2002). More specifically, the rate of dissociation for MG001 is 10-fold faster in Applicants' assay while that of BIRB-796 is 10-fold slower. Applicants believe that these differences are a consequence of the type of assay used by Pargellis et al., in which the dissociation of pyrazolourea compounds is measured by using p38α-specific ATP competitive inhibitor, SKF86002, as a displacer. Upon binding, SKF86002 becomes fluorescent thereby providing a way to monitor BIRB-796 dissociation. However, this inhibitor has a Kd ˜180 nM (Pargellis et al., 2002) and would therefore more effectively compete with 1, RL8 (published Kd ˜1.16 μM) than BIRB-796 (published Kd ˜0.1 nM) by shifting the activation loop toward the DFG-in conformation. Since Pargellis et al., calculate K_{d} from kₒₙ and k_{off}, the inefficient displacement of BIRB-796 by SKF86002 would result in a lower apparent K_{d} since calculated K_{d} values are more subject to the conditions under which the rate constants are obtained.

More recent measurements were performed using a new preparation of ac-p38α, BIRB-796 and 1, RL8 to make several repeated measurements of the dissociation (see FIG. 3 and Table 5 below) kinetics of each inhibitor. The k_{off} was found to be 5.1±0.5×10⁻⁵ s⁻¹ (n=3) for BIRB-796 and 7.1±3.2×10⁻³ s⁻¹ (n=3) for MG001. As described also above, in the case of BIRB-796, the k_{off} differs by a factor of 10 from those published elsewhere for BIRB-796 using alternative methods and assaying conditions (Pargellis et al., 2002). In the case of 1, RL8, the k_{off} differs by a factor of 100 from previously reported values (Pargellis et al., 2002). Differences in the rate constants shown in Example 4 together with FIG. 3 and here in Example 7 may be explained by the different ac-p38α protein preparations used. Differences in the rate constants determined using ac-p38α and the methods of (Pargellis et al., 2002) are explained by the different assay systems (SKF86002 competition assay) and conditions used to obtain the rate constants, as described above.

### Example 8

### Kinetics—Determination of kₒₙ

After measuring k_{off} for allosteric compounds, Applicants established conditions for measuring the kₒₙ of the same compounds. This was accomplished using the cuvette method by adding various concentrations of inhibitor to ac-p38α and fitting the fluorescence decay to a first-order function. The observed rate constant (k_{obs}) of the fluorescence decay for a specific dose of inhibitor was then plotted against the inhibitor concentration. Under the established conditions, inhibitor was added in molar equivalents to ac-p38α (1-4:1 inhibitor:protein) and the result is typically a straight line which can be fitted linearly with a R²>0.99. These conditions are similar to those used elsewhere for determining kₒₙ of a ligand to a protein with a single binding site (Hibbs et al., 2004). The slope of this line gives kₒₙ for the binding of the inhibitor to the kinase. The determination kₒₙ for BIRB-796 and 1, RL8 is shown in FIG. 6 (RL8 is called MG001 in the figure).

These measured kₒₙ values are different from those published by Pargellis et al. by a factor of 100 for both BIRB-796 and 1, RL8 (Pargellis et al., 2002). However, as reported in that study, the kₒₙ for these two inhibitors are very similar to each other. The differences in their K_{d} values is attributed primarily to differences in k_{off}, as Applicants also observed and described above. More recent measurements were performed using a new preparation of ac-p38α, BIRB-796 and MG001 to make several repeated measurements of the binding (see Table 5 below). The kₒₙ was determined to be 4.3±0.8×10³ M⁻¹s⁻¹ (n=3) and 6.6±1.2×10³ M⁻¹s⁻¹ (n=3) for BIRB-796 and 1, RL8, respectively. As with the newest measurements of k_{off} described in Example 7, the kₒₙ values for BIRB-796 and 1, RL8 differ by 10 and 100-fold, respectively, from values obtained elsewhere using the SKF86002 displacement assay (Pargellis et al., 2002).

Interestingly, Applicants were also able to perform the SKF86002 displacement assay for BIRB-796, but were unable to duplicate the kₒₙ values obtained by Pargellis et al. However, using similar conditions to Applicants' ac-p38α assay (amount of protein and inhibitor, buffer, temperature and mixing conditions) and using the same ratios of p38α to SKF86002, Applicants obtained a kₒₙ from the SKF86002 assay of 1.00×10³ M⁻¹s⁻¹, which is very well comparable to the value obtained using ac-p38α. This highlights the issue described in Example 7 regarding the use of different assay systems for determining rate constants for ligand binding and dissociation.

Determination of kₒₙ and k_{off} also allows for the indirect/calculated determination of K_{d} values (K_{d}=k_{off}/kₒₙ) and sheds light on the factors contributing to different ligand affinities. Using the rate constants for binding and dissociation that were measured directly using ac-p38α, Applicants obtained a calculated Kd for BIRB-796 and 1, RL8 of 10.2 nM and 1.16 μM, which are in strong agreement with the values Applicants obtained through endpoint measurements under similar conditions. Furthermore, Applicants' newest reported results obtained with a fresh preparation of ac-p38α yield calculated Kd values for BIRB-796 and 1, RL8 of 11.9±1.3 nM and 1.079±0.347 μM, respectively. By reaching similar results through two different methods and with different ac-p38α preparations using the described invention, Applicants are confident that the fluorescent-labeled kinase approach can not only provide accurate K_{d} measurements, but also valuable kinetic information about binding and dissociation from the protein.

The optimal method for accurately measuring the affinity of any ligand to a protein is to directly measure the formation of the ligand-protein complex as demonstrated with Applicants' approach. However, Applicants were still able to observe that the K_{d} values of different pyrazoloureas were more influenced by k_{off} rather than kₒₙ, an effect which is well-documented in the case of p38α (Pargellis et al., 2002).

### Example 9

### Detection of Potent ATP-Competitive Inhibitors/Identifying False Hits in Screens

In addition to several allosteric inhibitors, Applicants have tested several known ATP competitive inhibitors of p38α in Applicants' assay. The majority of compounds, including ATP, did not generate any kind of fluorescence change upon binding to ac-p38α. However, in the case of the most potent inhibitors (Kd ˜1-20 nM), the fluorescence change was robust, allowed binding curves to be generated and the resulting Kd values were comparable to the published values for binding to p38α. However, compounds which bind to p38α with a K_{d}>20 nM, the K_{d} values measured in Applicants' assay begin to diverge quickly from the published values. A few examples are shown in Table 2.

A simple experiment was designed to confirm that the fluorescent-tagged kinase loses the ability to accurately sense the binding of ATP-binding compounds with Kd >20 nM.

Applicants used the ATP-competitive inhibitor described above, SKF86002, to make endpoint measurements and obtain a binding curve with a Kd ˜78 nM using the intrinsic fluorescence of the inhibitor which is produced upon binding to the ATP-binding site of ac-p38α. This value is actually slightly lower than the published value of 180 nM (Pargellis et al., 2002). Since the fluorescence of SKF86002 is measured at 420 nm, there was no interference from acrylodan in these measurements. Using an alternative approach, Applicants used the same protein and inhibitor samples and generated a titration curve based on acrylodan fluorescence. In this case, the Kd value obtained was 10-fold higher (Kd ˜721 nM). Therefore, it is clear that the acrylodan label itself is insensitive to most type I inhibitors most likely due to the fact that the fluorescent-tagged activation loop is not expected to change conformations upon binding of these types of ligands. Accordingly, Applicants' assay not only detects all allosteric binders which induce the DFG-out conformation of the kinase, it can also report the Kd for some tight binding ATP-competitive inhibitors. An interesting finding was that the promiscuous ATP-competitive kinase inhibitor staurosporine was not detected by Applicants' assay. In fact, it was reported that p38α is one of the few kinases that staurosporine does not inhibit (Karaman et al., 2008).

TABLE 2
ATP-competitive inhibitors tested in the ac-p38α assay.

| | | | Pub. Kd | |
|---|---|---|---|---|
| Name | Compound | Kd (nM) | (nM) | Ref. |
| SB203580 | 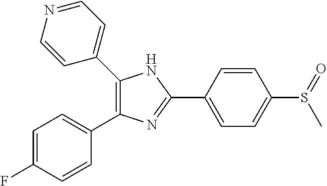 | 15 | 9-20 | |
| Dasatinib | 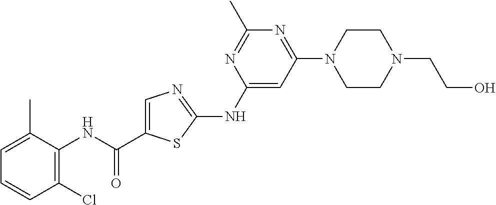 | 389 | 27 | |
| SKF86002 | 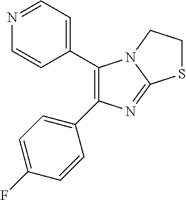 | 721 | 180 | |
| Ro3201195 | 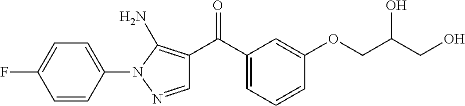 | 1,914 | 95-170 | |
| Staurosporine | 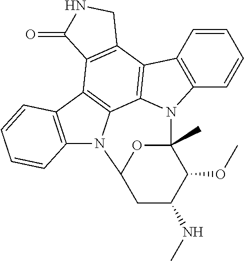 | Not Detected | >10000 | |

Applicants are currently looking at structural information to understand how the fluorescent labeled kinase can sense these compounds, particularly when there is no conformational change in the activation loop and the inhibitor is not close enough to the fluorophore to directly alter its fluorescence. The most likely explanation is that some ATP-competitive inhibitors induce another kind of conformational change in kinases upon binding which could cause a slight shift or rotation in the position of the acrylodan without affecting the activation loop. The slightest shift into a more polar or charged environment could be enough to change the fluorescence. In particular, Applicants will continue to examine structures to determine how the Kd of SB203580 is accurately reported by ac-p38α, with a special focus on ATP competitive compounds which may form an interaction with the Asp of DFG or compounds that approach or enter the small hydrophobic sub-pocket in the vicinity of the gatekeeper residue of p38α. These structural investigations are close to completion.

Applicants have determined that the binding of some Type I inhibitors may induce unexpected conformational changes involving the activation loop and/or a reorientation of the N-lobe relative to the C-lobe upon binding to the kinase hinge region. In the latter case, these localized conformational changes could modulate the polar environment in the vicinity of the fluorophore without movement of the activation loop, resulting in false hits when screening for allosteric binders. Applicants identified one such compound, SB203580, which is a potent low nM Type I p38α inhibitor and close structural analog of SKF86002. Surprisingly, the fluorescence change observed in ac-p38α upon binding of SB203580 was robust and allowed binding curves to be generated (FIG. 28B) which gave K_{d} values that were the same as those previously published using other methods (15±2 nM) (Regan et al., 2002).

In order to better understand why this compound in particular triggered such a sensitive response, Applicants co-crystallized it with p38α (FIG. 28C). The structure was solved to a resolution of 2.3 Å with positive difference density for the inhibitor clearly visible in the ATP binding pocket. Although the pyridinyl group of the inhibitor forms a hydrogen bond to the hinge region, which qualifies SB203580 as a Type I inhibitor, the kinase adopted the DFG-out conformation. The plane of the methylsulfinyl-substituted phenyl is sandwiched between the DFG motif and the P-loop and forms π-π stacking interactions with the side chains of Tyr35 (P-loop) and Phe169 (DFG motif). The N3 of the imidazole moiety hydrogen bonds via a water molecule to the backbone of Leu167 located at the N-terminal end of the DFG motif. The net result of these interactions is the stabilization of p38α in the DFG-out conformation despite the Type I binding mode of SB203580. Although the compound is not bound within the allosteric site, the assay detected the compound due to this unique binding mode.

Interestingly, SB203580 has been analyzed extensively by both protein X-ray crystallography and NMR techniques (PDB codes: 2ewa; Vogtherr et al, 2006) and 1a9u, Wang et al., 1998). While one study reported the binding of SB203580 to the DFG-in conformation, Wang et al., 1998, the other group reported that this inhibitor can in fact bind to both DFG-conformations (˜50% inhibitor occupancy in each conformation) and further confirmed this finding using 2D-NMR experiments.

Using the intrinsic fluorescence of the high affinity ATP-competitive inhibitor of p38α, SKF86002, to measure its binding as described by (Pargellis et al., 2002), Applicants found that the inhibitor binds to ac-p38α with a K_{d} ˜78 nM. However, performing the same experiment while monitoring changes in the ratiometric fluorescence of acrylodan results in a K_{d} of 721 nM, highlighting the above described insensitivity of acrylodan to Type I inhibitors. Applicants can postulate based on these observations that SKF86002 may bind in a similar manner to SB203580. Both compounds share structural similarities, in particular the 4-fluorophenyl moiety which likely extends back into the hydrophobic sub-pocket behind the gatekeeper as observed for SB203580. The core Y-shaped structure of these compounds is exactly the same, with the exception of the additional phenyl moiety of SB203580 which is responsible for forming the π-π stacking interactions with Phe169 and Tyr35 of p38α to stabilize the DFG-out conformation. This ring is not present in SKF86002. Assuming the binding mode is similar, this structural difference may reduce the ability of SKF86002 to stabilize the DFG-out conformation, thus explaining the observed insensitivity of the acrylodan-labeled activation loop to its binding, in contrast to its highly sensitive response to SB203580. Regardless of this insensitivity to most Type I inhibitors, the assay appears to be very sensitive to Type I binders that interact and modulate the conformation of the DFG motif and, thus, the activation loop.

As a result of these findings, it is likely that a few ATP-competitive inhibitors may register as false hits while screening for allosteric inhibitors. Therefore, it was necessary to determine whether or not ATP and allosteric compounds could be discriminated from one another using Applicants' assay.

Using the cuvette method, Applicants were quickly able to accomplish this by looking at the kinetics of the fluorescence changes. In the case of allosteric inhibitors such as BIRB-796, Applicants have already shown that the kinetic is relatively slow and the fluorescence change takes several minutes to reach completion (see FIG. 7B, FIGS. 3B and D). However, in the case of ATP-competitive inhibitors such as SB203580, the induced fluorescence change is instantaneous (2-4 sec). This is not surprising since a protein conformational change is not required to allow binding of these compounds as is the case for allosteric inhibitors. Further, the ATP binding site is relatively easy to access in comparison to the allosteric site which only becomes available when the kinase samples the DFG-out conformation. This instantaneous response was also observed for Ro3201195 and SKF86002. An example of these results is shown in FIG. 7.

### Example 10

### 384-Well Plate Format

The endpoint assay described for cuvettes for measuring the Kd of allosteric inhibitors has now been scaled down for use in a 384-well and 96-well plate formats with 20 μl and 100-200 μl total drop volumes for these plate types, respectively. Care was taken to improve inhibitor solubility and to limit the number of dilution and pipetting steps. Results for BIRB-796 are shown in FIG. 8.

For 384-well plates, inhibitor stocks were prepared in DMSO at 20× the final desired concentration. Each well contained 1 μl of inhibitor solution+19 μl of buffer containing 50 nM ac-p38α (5% v/v DMSO after mixing). The buffer is the same as that used in the cuvette method with the addition of 0.01% v/v Brij-35 or Triton X-100, a standard detergent used to improve inhibitor solubility. Under these conditions, no visible precipitation of BIRB-796 was observed. At this time, repeated screens have been performed with the inhibitor BIRB-796 to optimize the signal-to-noise ratio, incubation time and incubation temperature. After mixing, an incubation time of 6 hrs at room temperature or overnight at 4° C. was found to be adequate to reach equilibrium and achieve the lowest measurable Kd for each inhibitor (Kd ˜70 nM for BIRB-796) and good signal-to-noise (Z factor ˜0.77). Using the cuvette method described above, Applicants were able to demonstrate the expected time-dependence of BIRB-796 inhibition of p38α (FIG. 9). In a further example, an HTS screening of a 34,000 compound library was performed using the assay. An excellent Z-factor of 0.85±0.06 was achieved across the complete screen which required 97 384-well plates.

Similar buffer and incubation conditions were used in a 96-well format. However, 200× stocks of inhibitors were prepared in DMSO and diluted into a total volume of 200 μl (0.5% v/v DMSO after mixing). In this format, the Kd for BIRB-796 was found to be ˜27 nM with slightly better signal-to-noise ratios than the 384-well format (Z factor ˜0.84). All measurements of the plates were made with a Tecan Safire2. Numerous additional screens have provided a more accurate assessment of the 96-well format (Z-factor=0.88±0.03)

Compared to the values obtained with the cuvette method, the measured Kds are 5-fold higher for the tightest binding inhibitor, BIRB-796, in the 96-well format and even higher in the 384-well format. Therefore, the HTS format most suitable for Kd estimation is the 96-well format while 384-well plates are the best for initial HTS screens of allosteric binders.

### Example 11

### Application of HTS-Format

Since Applicants' initial report, the 384-well format has been used to make endpoint measurements in a compound library screen. The molecules screened were designed using computer simulations and modeling, to bind to the DFG-out conformation of kinases. The DFG-out conformation is the conformation required for allosteric inhibitor binding which is detected by this assay. The core structure of these compounds and their proposed binding mode is shown in FIG. 10.

All compounds share a 2,5-disubstituted thiazole moiety with a urea or amide in the 2 position to generate similar interactions with the kinase as the classic p38α pyrazolurea compounds. The thiazole moiety was designed to be positioned near the small hydrophobic sub-pocket into which the naphthalene moiety of BIRB-796 is bound in p38α and result in the proper positioning of the amide or urea moiety to make the characteristic interactions made by pyrazolourea compounds for strong binding to this pocket. Similarly, bulky hydrophobic moieties were placed after the urea/amide position to better occupy the allosteric pocket. The opposite end of the molecules were decorated with various alkyl moieties and/or phenyl rings along with polar hydroxyl groups, amines and N atoms which could form H-bonding interactions in the more polar adjacent ATP-binding pocket. Compounds of varying size were generated to create a library of potential Type III (exclusively allosteric) and Type II (bridged between the allosteric and ATP sites) binders.

For this screen, a pipetting scheme was generated in which 4 dilutions of each library compound were prepared in 384-well plates using DMSO as the solvent. Each dilution was 20× the desired final concentrations (0.05, 0.5, 5, 50 μM) used in the screen. The pipetting scheme for the assay was as described above for 384-well plates with the exception that the amount of ac-p38α was increased to 100 nM to avoid any background fluorescence from the compounds which may be present at high concentrations (500 μM). BIRB-796 was used as positive allosteric binding control and ac-p38α without inhibitor was used for background/baseline fluorescence. After mixing, the plates were incubated at RT for 5-6 hrs before measurement with a Tecan Safire².

The screen identified 11 compounds which all increased the fluorescence ratio of ac-p38α at a concentration of 500 μM. This corresponds to a 1.8% hit rate. However, only 5 of these hits bound stronger than the remaining 7 compounds. No compound generated a fluorescence change as significant as that of BIRB-796, suggesting that all hits are weaker binding compounds. Interestingly, the structures of these compounds shared a similar feature in the region of the molecule which was proposed to bind in the ATP site, leading us to propose that the binding mode of these compounds is actually flipped 180° from the proposed mode. This particular moiety may give the compounds a favorable interaction with the protein and may induce the DFG-out conformation and the fluorescence response.

The next step was to verify the screening results and to assess the binding mode of these compounds (ATP-competitive vs. allosteric) as described above using the cuvette method. All hits produced the correct changes in emission spectra (FIG. 2) and the 5 strongest hits had Kds ranging between 14-40 μM. The remaining hits from the large library screen had Kds of 40-60 μM. It is important to note that the Kd values for these compounds are rather high, which is likely due to the fact that each compound has a stereocenter and was present as an enantiomeric mixture in the library as provided by the manufacturer. Enantiomerically pure hits would very likely have a lower Kd. However, despite this effect, the assay was still able to identify hits which share similar structural features. Characterization of one compound (85-C8) is shown in FIG. 11.

Addition of each hit (a 30 μM single dose) to ac-p38α produced a new and surprising kind of fluorescence response which resembles a mixture of the responses seen in FIG. 7 by SB203580 and BIRB-796. Upon addition of the compound, there is an instantaneous decrease in fluorescence, which is indicative of binding in the ATP pocket, followed by a slow fluorescence decay that can be fit with a first order function, which is indicative of movement of the activation loop and access to the allosteric site. Although unexpected, the magnitude of the instantaneous fluorescence change and the kinetics of the slow phase of fluorescence change are both dose-dependent and can be fit linearly when plotted against inhibitor concentration.

Based on these initial fluorescence results, Applicants returned to the idea described above about whether or not these hits actually have a flipped binding mode. A recent publication provided us with evidence that this flipped binding mode is certainly possible (Andersen et al., 2008). In Aurora kinase, the binding mode of dasatanib (Sprycel), a Src/Abl kinase inhibitor, and INH-29 are predominantly ATP-competitive and bind to the hinge region of the kinase. Binding to this region is a prerequisite to strong ATP-competitive inhibition of kinases. Both compounds, INH-29 in particular, share many structural features with Applicants' library hits. Both are 2,5-disubstituted thiazoles and INH-29 also has a urea moiety in the 2 position. Using this crystal structure of dasatinib, Applicants modeled in on of the library hits and overlaid it onto dasatinib and found great alignment of many pharmacophore N atoms in the structures. This structure allowed us to construct a model of the proposed binding mode of Applicants' hits, in which the conserved chemical moiety of these compounds is just long enough to extend from the ATP binding site into the allosteric pocket. The comparison of Applicants' hits with dasatinib is shown in FIG. 12.

Thus, the data combined with Applicants' models may suggest that the hits bind rapidly to the hinge region of the kinase in a manner similar to dasatinib. Once bound, the conserved structural moiety of the hit compounds might slowly position itself in part of the allosteric pocket and trigger the slow fluorescence change which follows the initial rapid response.

Real-time fluorescence measurements of each of the strongest hit compounds seem to support this idea (data not shown). Addition of a single dose (30 μM) of each hit compound to a cuvette containing ac-p38α results in fast fluorescence changes of variable magnitudes. However, the k_{obs} of the slow phase of fluorescence change is in a similar range for all hit compounds (4.2-8.8×10⁻⁴ M⁻¹s⁻¹). Dasatinib lacks a moiety which can reach the allosteric pocket when bound in this mode, and in Applicants' cuvette assay, dasatinib only produces an instantaneous fluorescence response which is characteristic of exclusively ATP-competitive inhibitors. Interestingly, titration of dasatinib with ac-p38α resulted in a Kd ˜389 nM. This is much higher than the reported Kd of ˜27 nM in p38α (Karaman et al., 2008), again suggesting that most of the inhibitor resides in the ATP pocket and is in line with Applicants' observations that acp38α can only accurately report the Kd of ATP-competitive inhibitors with Kd <20 nM.

Several follow-up studies on these compounds have been performed to further characterize their mode of binding and to validate these hits as inhibitors of kinase activity. Applicants first validated the findings of Applicants' HTS screen using the labeled kinase binding assay by testing the compounds in a commercially available activity-based assay. The affinity of these compounds is fairly weak (mid μM Kd values) but they exhibit the same activity in inhibiting p38α kinase activity (mid μM IC₅₀ values). These data are shown in tabular form in FIG. 12 D.

To better predict the binding mode of these thiazole compounds, Applicants again used acrylodan-labeled p38α to measure the binding kinetics in real-time. However as opposed to Applicants' earlier measurements, Applicants used only 2 μM of each compound. The rationale for using less compound than that used to study kinetics in FIG. 11 is that high amounts of added ligand increase the rate of binding and this might explain the initial rapid phase described above. As expected, using only 2 μM eliminated this initial fast kinetic leaving only the slow kinetic phase. Applicants believe that this more clearly indicates binding in the allosteric pocket as opposed to the dasatinib-like binding mode Applicants initially proposed in FIG. 12. In the case of 87H9, the binding rate was very slow (t_{1/2}=118 sec). All other thiazole-urea hit compounds behaved similarly (data not shown).

To confirm and validate the predictions made by Applicants' binding assay with regards to the binding mode, Applicants co-crystallized several of these compounds with wild type p38α but only were able to obtain the crystal structure for 87H9 (FIG. 12E). The ligand is indeed located completely within the allosteric pocket and is bound to the DFG-out conformation. The phenyl moiety described above in Applicants' previously modeled binding mode (see FIG. 11) is buried inside the hydrophobic subpocket located beyond the gatekeeper residue. This structural feature is conserved in all thiazole-urea compounds identified in this screening initiative and is likely a crucial contributor to their affinity. Additionally, the urea moiety forms the expected interactions with the DFG motif and a glutamate side chain of the C-helix. These hydrogen bonding interactions are characteristic for ligands with a urea moiety which bind in the allosteric pocket. The identification of these thiazole-urea compounds as ligands which bind within the allosteric pocket of p38α represents a novel binding mode for this class of compounds, which are typically Type I inhibitors which compete with ATP.

### HTS Screen of a 35,000 Compound Library

### HTS Screen Summary

Applicants screened a large collection of compound libraries, consisting of approximately 35000 compounds using the acrylodan-labeled kinase binding assay for p38α described in this application (see FIG. 12 F for a scheme). The kinase is labeled on the activation loop in order to identify ligands which specifically bind to and stabilize the inactive DFG-out conformation. DFG-out binders induce a shift in the emission maximum from 468 nm to 514 nm and the dual emission maxima allow for ratiometric measurements of ligand binding to be made at equilibrium (endpoint measurements). These types of ratiometric fluorescence readouts are advantageous since they correct for small dilution and pipetting errors between different samples in a titration series and eliminates “edge effects” which are frequently observed in small volume HTS plates.

The complete screen was carried out by first using the labeled kinase binding assay in a 384-well HTS format to initially screen for possible ligands for the DFG-out conformation, or DFG-out binders. This was accomplished by first performing a primary screen at a single concentration of each ligand, followed by a secondary screen over a range of concentrations to directly determine the Kd of each potential hit.

HTS Screen Setup for Acrylodan-Labeled p38α.

The methods for the setup and execution of the screen are provided below. The primary screen was carried out using a single concentration (12.5 μM) of each ligand to first determine which compounds induce and stabilize the DFG-out conformation of p38α. Pre-stocked inhibitor plates (1 compound per well at 10 mM in DMSO) were used to first prepare pre-dilution plates by diluting compounds from the stock plates to 50 μM in buffer (50 mM Hepes pH 7.45, 200 mM NaCl, 0.01% Triton-X100 (Note: Brij-35 may also be used in place of Triton)). Large volumes of the same buffer were also use to prepare solutions for pipetting background (no labeled kinase added) and screening plates (+100 nM acrylodan-labeled p38α).

An industrial pipetting robot was used to first dispense 50 of pre-diluted compounds into a set of two 384-well small volume assay plates. Subsequently, 15 μl of buffer was added to the background plate while the same volume of buffer containing the labeled kinase was added to the screening plate to detect DFG-out binders. Both plates were covered with adhesive foil and stored at 4° C. overnight since DFG-out binders have notoriously slow association rates in p38α (Pargellis et al. 2002) The % v/v DMSO was <0.2% in all plates. A Tecan Safire² instrument was used to measure the fluorescence read-out in the 384-well plate format. All plates also contained 6 wells of negative DMSO control (no ligand) as well as 6 wells of positive control (12.5 μM BIRB-796). Data was processed by subtracting intrinsic compound fluorescence at 514 nm and 468 nm (background plate) from the signal measured in presence of acrylodan-labeled p38α (screening plate). Background plates corrected for intrinsic compound fluorescence and eliminated a large percentage of the most highly-fluorescent compounds in the library. In most cases, background corrected ratiometric fluorescence values of such compounds were the same as the negative DMSO control (data not shown). As described previously, the extent of binding was then assessed by taking the ratiometric fluorescence (R=514 nm/468 nm) of the background-corrected data. Any compound which reached 25% of the maximal response observed for the positive control was submitted to subsequent testing.

A secondary screen was carried out also in 384-well plates using a range of concentrations (100 nM to 50 μM) of each ligand in order to generate binding curves or identify false hits which were picked up due to high degrees of fluorescence interference. Pre-dilution plates were prepared using buffer such that concentration of compound was 2-fold higher than that needed in the final screening plate. As before, large volumes of the same buffer were also used to prepare solutions for pipetting background (no labeled kinase added) and screening plates (+100 nM acrylodan-labeled p38α). The pipetting robot was used to first dispense 3.5 μl of pre-diluted compounds into a set of two 384-well small volume assay plates. Each plate contained no more than 7 different compounds identified in the primary screen, each screened at 10 concentrations (100 nM to 50 μM) and 4 wells per concentration. Subsequently, 3.5 μl of buffer was added to the background plate while the same volume of buffer containing the labeled kinase was added to the screening plate. Plates were sealed, incubated and measured as described for the primary screen. Raw data at 514 nm and 468 nm as well as background-corrected ratiometric data were used to eliminate false fluorescent hits. An exemplary sample plate layout is shown below.

Ratiometric fluorescence values enabled reliable binding curves to be plotted to directly determine the K^{d} of ligand binding to p38α. Where indicated, binding curves were also plotted as % p38α bound by the ligand. The % p38α bound is calculated as follows:

% bound=((R−Rᵤₙₛₐₜ)/R_{sat'd})×100

where R is the ratiometric fluorescence at a given concentration of ligand and R^{unsat} and R^{sat'd} are the ratiometric fluorescence values obtained for p38α in the absence or presence of a saturating concentration of the same ligand, respectively.

### Hit Identification and Validation

The performance of the primary assay screen was assessed by monitoring the ratiometric values of the positive and negative controls of all plates and yielded a calculated Z-factor of 0.82±0.6 for the entire screen (FIG. 12G). After the first round of screening, 90 compounds were identified as potential hits, corresponding to a “hit rate” of only ˜0.25%.

Compounds which gave sigmoidal binding curves in the secondary screen were confirmed as likely DFG-out binders while any remaining highly-fluorescent compounds were easily identified as false hits. Changing ratiometric fluorescence values were used to plot binding curves and directly determine Kd values. After two rounds of screening, only 35 compounds remained were confirmed as likely DFG-out binders.

In the next validation step, all compound stocks were analyzed by LC-MS to assess the purity and to verify the expected compound mass. Only compounds that were found to be >80% pure were subjected to further screening with the HTRF kinase assay (commercially available from CisBio) for IC₅₀ determinations, according to the manufacturer's instructions Following completion of these follow-up validation studies of the initial 35 hits identified as being DFG-out binders using acrylodan-labeled p38α, 27 of these compounds also inhibited enzymatic activity of p38α in the HTRF assay as validated kinase inhibitors.

Several compounds (HTS 1-15) as well as determined Kd and IC₅₀ values are presented in Table 3. In many cases, the Kd determined using acrylodan-labeled p38α is in close agreement with the IC₅₀ values determined in activity-based assays, validates the use of a non-phosphorylated inactive p38α for identifying DFG-out binders capable of inhibiting the active phosphorylated kinase, which is required for activity-based assays. However, compounds HTS 3-6 and HTS 12 are 10 to 50-fold less active in the activity-based assay. The loss of affinity for inhibitors which bind partially within the allosteric pocket adjacent to the ATP binding site is well documented (Seeliger et al., 2007). The phosphorylation of the activation loop of p38α, which is required for the activity-based HTRF assay, likely stabilizes the DFG-in conformation of p38α. If their binding mode is dependant on the DFG-out conformation, this explains their significantly higher IC₅₀ values.

Thus, by utilizing the non-phosphorylated form of the kinase for Applicants' assay system, the DFG-out conformation is energetically more favorable and likely enhances sensitivity for the detection of DFG-out binders in large compound libraries. The increased sensitivity to HTS 3-6 and HTS 12 demonstrates a key advantage to using the labeled kinase binding assay for HTS screening of kinase inhibitors which bind to this inactive kinase conformation.

Interestingly, compounds HTS 1 and HTS 2 are derivatives of the potent Type I p38α inhibitor SB203580 (FIG. 7). The binding mode in p38α is well described and is unique in that the inhibitor retains a Type I binding mode but is able to bind to and stabilize the DFG-out conformation of p38α by forming π-π interactions by stacking between the side chain of the DFG Phe (Phe169) and the side chain of a Tyr residue (Tyr35) found in the glycine-rich loop as described in Example 6 of this application. Therefore, the detection of HTS 1 and HTS 2 using Applicants' novel binding assay served as an internal validation of the results. Given their high affinity and inhibitory activity, 1 and 2 likely adopt the same Type I binding mode in p38α.

### Binding Kinetics and SAR of Selected Hit Compounds

Since this assay also detects Type I ligands which stabilize the DFG-out conformation, such as SB203580, Applicants performed real-time kinetic measurements of the binding of these compounds to acrylodan-labeled p38α to get some insight into the possible binding mode of these hits.

Two hits from the HTS screen, HTS 14 and HTS 15, were not commercially available for testing in an activity-based assay. Therefore, several close derivates (HTS 14a-e and HTS 15a-c) were obtained for IC₅₀ determinations and for the purposes of performing SAR studies (FIG. 12H). The acrylodan-labeled p38α binding assay was used to determine the Kd of each compound and Applicants found a clear preference for compounds with a meta-substituted phenyl ring, more specifically, a halogen substituent at this position. Replacement of the meta-chlorine of HTS 14a with a meta-bromo in HTS 14b results in a 100-fold reduction of the Kd. Furthermore, a comparison of HTS 14c and HTS 15a reveals that replacement of the cyclohexyl with a cyclopentyl ring reduces affinity by nearly 10-fold. As was the case with several other hit compounds, these derivatives have a much weaker effect in the activity-based assay.

TABLE 3
Kd and IC₅₀ values of compounds HTS 1-15.

| Compound | Kd (μM) | IC50 (μM) |
|---|---|---|
| 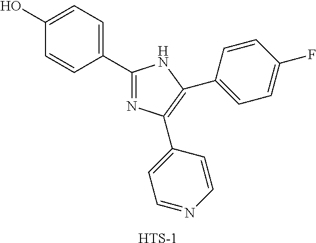 | 0.117 ± 0.009 | 0.022 ± 0.009 |
| 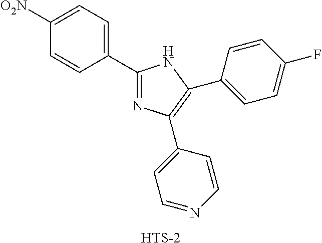 | 0.179 ± 0.019 | 0.022 ± 0.006 |
| 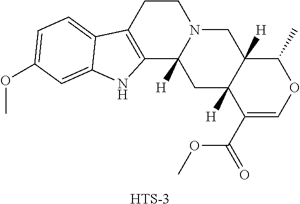 | 2.20 ± 0.11 | 18.3 ± 7.2 |
| 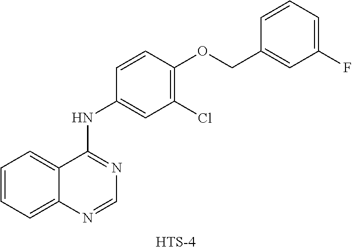 | 10.3 ± 2.2 | >50 |
| 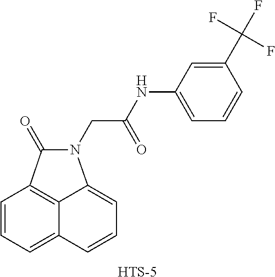 | 3.10 ± 0.91 | >50 |
| 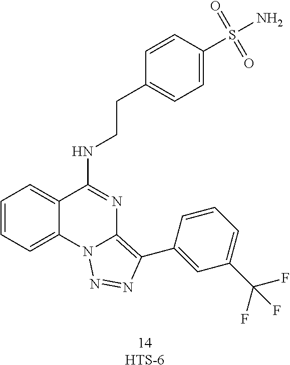 | 0.301 ± 0.059 | >50 |
| 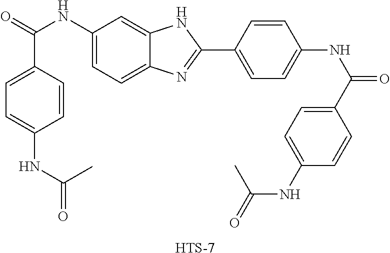 | 6.18 ± 2.31 | 15.3 ± 2.8 |
| 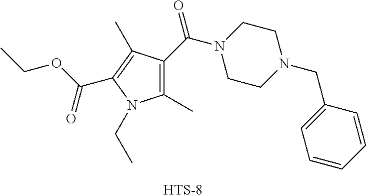 | 3.29 ± 0.64 | 2.6 ± 0.9 |
| 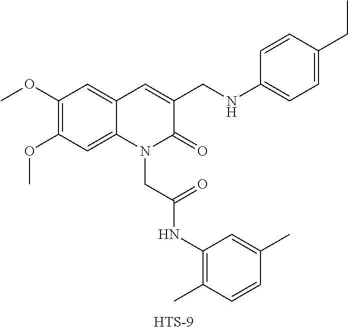 | 24.9 ± 4.9 | 25.6 ± 4.7 |
| 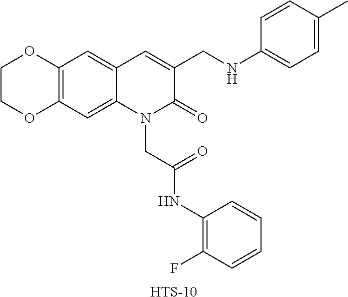 | 29.4 ± 4.0 | 31.3 ± 17.2 |
| 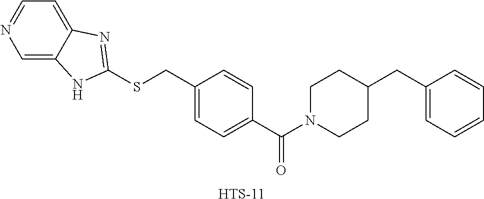 | 2.30 ± 0.57 | 2.60 ± 0.92 |
| 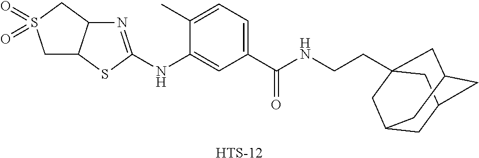 | 1.30 ± 0.08 | 11.0 ± 1.5 |
| 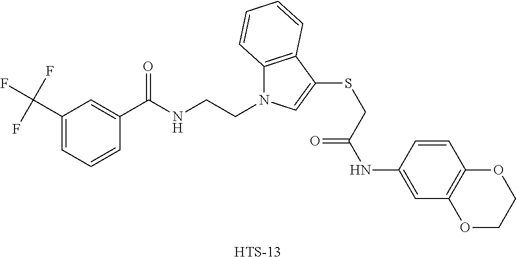 | 0.839 ± 0.145 | 1.10 ± 0.40 |
| 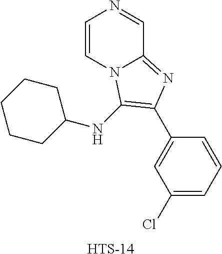 | 100.4 ± 5.8 | N/A |
| 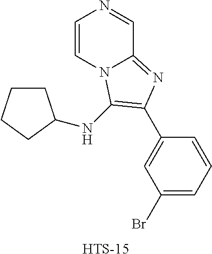 | 27.5 ± 5.3 | N/A |

So far, co-crystallization of these compounds with p38α have been unsuccessful and a detailed understanding of the binding mode is not yet possible. However, using acrylodan-labeled p38α, Applicants were able to perform endpoint measurements to obtain binding curves for the commercially available derivatives HTS 14b-d (FIG. 12H lower left). Real-time kinetic measurements of HTS 14b reveal a rapid decrease in the fluorescence emission of acrylodan at 468 nm, indicative once again of the binding of a Type I ligand which may somehow induce and stabilize the DFG-out conformation of the kinase. Similar data were also obtained for the well-known p38α inhibitor, SB203580, using this assay system (see FIGS. 7 and 28).

### Structural Details of Ligand Binding

Kinetic measurements of ligand binding (see FIG. 12I) suggested that nearly all compounds were likely to be Type I inhibitors with the exception of HTS 12, which gave a clear slow binding kinetic (t_{1/2} ˜38 sec). An example of a fast binding compound is also shown that is indicative of binding to the DFG-out conformation of p38α. This type of slow binding kinetic is characteristic of Type II/III ligands which bind completely or partially within the allosteric pocket. Regardless of the binding mode predicted by the real-time kinetic measurements, the fact that they were sensitively detected by Applicants' novel binding assay suggests that they may somehow stabilize the inactive DFG-out conformation.

Applicants attempted to co-crystallize several of the remaining inhibitors with wild type p38α in order to obtain detailed structural information about the binding mode and to validate the kinetic information obtained from real-time measurements of ligand binding to acrylodan-labeled p38α (FIG. 12J). Several compounds either did not co-crystallize with the protein or yielded crystals in which the inhibitor occupancy was too poor to model in the compound properly. Despite these difficulties, Applicants were able to obtain protein X-ray crystal structures of HTS 8 and HTS 11-13 in complex with p38α. The crystal structure of HTS 12 reveals that the ligand is bound within the allosteric site adjacent to the ATP binding site and that the kinase is in the DFG-out conformation, in agreement with the slow kinetics of binding observed in Applicants' kinetic measurements with acrylodan-labeled p38α (data not shown). However, the electron density of the ligand was not good enough to properly model in all parts of the inhibitor.

The best crystal structure obtained was for HTS 13, which binds in a Type I binding mode, as suggested by the kinetics measurements, but the activation loop of p38α is found in an inactive conformation. The DFG Phe side chain appears to be pulled deep into the ATP binding pocket where it interacts with a hydrophobic patch on the side of the inhibitor molecule. This patch appears to be generated by an internal hydrogen bond which allows the inhibitor to form a coil within the ATP binding site and presents a large hydrophobic patch which faces the direction of the DFG Phe side chain. Additionally, the inhibitor contains a trichlorophenyl moiety which extends beyond the gatekeeper residue into a hydrophobic subpocket. Moieties binding within this subpocket are known to enhance potency of ligands for p38α and may also shift the conformational equilibrium of p38α such that the DFG-out conformation becomes more energetically favorable (Regan et al., 2002).

### Example 12

### Labeling Strategy; Selection of a Labeling Site Using Sequence of Structural Data

In order to effectively demonstrate the feasibility of this approach in other kinases, Applicants have selected a series of Serine/Threonine and Tyrosine kinases from various species as the next candidates for this assay system. Most importantly, Applicants have chosen a series of kinases which are known to be regulated by the DFG-in and DFG-out conformational switch and for which some structural information is available either in one or both conformations. Applicants have also chosen kinases for which it is still unknown whether or not they can adopt the DFG-out conformation. For these kinases (GSK3β (human), GSK3 (fungal homolog), cSrc, CSK, EGFR (human), Lck (human) and Aurora A (human)), sequence alignments of the activation loop were generated using the DFG motif and a highly conserved APE motif as the start and end points of the loop, respectively. Applicants also aligned the sequences of kinases for which no structural information is yet available (DDR1 (human) and pfMAPK1 (Plasmodium falciparum)). The alignment of these kinases is shown in FIG. 13.

In p38α, Applicants attached the fluorophore two positions after the DFG motif (occupied by an alanine) by mutating this position, which is often a highly conserved alanine or serine, with a cysteine.

This site was chosen because it sits between the highly conserved DFG motif (Box 1 of FIG. 13) and the remainder of the activation loop, which contains numerous potential phosphorylation sites and various charged and/or hydrophobic residues involved in organizing the tertiary structure of the loop. All attempts were made to avoid these regions of the loop while also keeping in mind that the largest fluorescence changes will come from distinct changes in environment (solvent accessibility) rather then the quantitative distance of fluorophore movement. Applicants will perform SASA (surface area solvent accessibility) calculations for the fluorophore provided structural information has been obtained for ac-p38α in both the DFG-in and DFG-out conformations. Thus far, Applicants have observed the fluorophore only in the DFG-out conformation.

The labeling position chosen for p38α is typically followed by, in most kinases, a basic amino acid such as Lys or Arg (Box 2) that is involved in either forming ionic interactions with helix C of the kinase or interacts with phosphorylated residues in other regions of the activation loop. In most kinases, this position is frequently followed by a few hydrophobic residues such as Leu or Ile (Box 3), then a few more charged residues involved in stabilizing the activation loop (Box 4), then a variable phosphorylation region containing serine, Thr or Tyr residues (Box 5). Near the end of activation loop, a highly conserved basic residue followed by one or two bulky hydrophobic planar residues such as Tyr and Trp, can be found (Box 6). This is usually followed by the highly conserved APE motif (Box 7) at the end of the loop.

Analysis of these alignments reveals that p38α has a particularly short (more compact) activation loop compared to most kinases. Therefore, structural information was more helpful in identifying the most analogous labeling position in other kinases. For each kinase aligned above, available structures were aligned with the active (PDB code: 1wbo) and inactive (PDB code: 1wbs) form of p38α. In all cases, the extra long activation loops reveal that the position labeled in p38α may not be the best position for kinases which have a longer activation loop. For these kinases, the position directly following the DFG motif, which is one position before the p38α labeling site, appears to align best structurally. In most kinases, a Leu is found in this position. In cases where no structural information is available, as with DDR1 and pfMAPK1, structural models based on known structural kinase templates were generated using online tools to assist with the identification of the labeling site and cysteine residues which are solvent exposed. Mutation of these cysteines into serine is critical to eliminating non-specific fluorescent labeling. This kind of information cannot be obtained easily by looking at a sequence alignment.

Other exceptional cases also exist, such as Aurora A kinase which has a Trp residue immediately following the DFG motif. In this case, the Trp residue does not appear to change position significantly during the transition to the DFG-out conformation. Given the planar ring structure of the fluorophore, the adjacent position in the amino acid sequence was also avoided for labeling to help prevent favorable or unfavorable interactions with the hydrophobic Trp residue. Thus, the third position following DFG was chosen in Aurora A (Val).

Another exceptional case is EGFR, which forms an alternate inactive state and does not seem to be regulated by the DFG switch. Inactive EGFR undergoes a conformational change of the activation loops which brings it more into the ATP binding site where it forms a mini a-helix. Although it may not be possible to screen for allosteric inhibitors in such a kinase, Applicants are attempting to use the same principals to label this kinase and screen for compounds which might induce this inactive conformation. Given the unique nature of EGFR, sequence alignments alone would not be enough to determine the best labeling site. Therefore, structural information was used to identify the position on the activation loop of EGFR which would relocate to a position similar to the labeled site of p38α in the DFG-out conformation. Applicants determined this to be a Leu which is five residues after the DFG motif. Several structural alignments are shown in FIG. 14 to illustrate these points.

Aside from a few exceptions, it seems the first two residues after the DFG motif are optimal for labeling the vast majority of kinases for use in this assay approach. This was confirmed in an alternative alignment in which a series of kinases different from those listed above were used to determine common motifs. The resulting alignment of (GSK3β (human), p38α (human), b-Raf (human), CDK2 (human), cSrc (human), CSK (human), EGFR (human), Lck (human), Abl (human) and Aurora A (human)) is shown in FIG. 13B. The activation loop sequence is bookended by the highly-conserved DFG (Box 1) and APE motifs (Box 6). The DFG+3 position is commonly a basic amino acid which interacts directly with the primary phosphorylation site of the activation loop (Nolen et al., 2004). The DFG+3 through DFG+5 serves as a hydrophobic anchor point with other structural features of the C-lobe in tyrosine kinases (Levinson et al., 2008). This is followed by a variable length segment (Box 3) and a region containing a high incidence Tyr, Ser and Thr residues which can be phosphorylated (Box 4). The C-terminal end of the activation loop (Box 5) forms several interactions with the C-lobe of the kinase and is important in substrate binding.

Applicants found that residues immediately following the DFG motif (DFG+1 and DFG+2) at the N-terminal end of the activation loop exhibit significant movement with conformational changes and are typically not associated with disease-related genetic alterations known to influence kinase activity (Torkamani et al., 2008). Additionally, sequence alignments of all human kinases reveal that at least 47 kinases have a naturally-occurring Cys in these two positions, suggesting that mutation of the labeling site residue to Cys would likely be tolerated.

As described above, Ala172 of p38α (DFG+2 position) was therefore subsequently mutated into a Cys for specific reaction with thiol-reactive fluorophores. Lastly, Applicants used available structural information to identify solvent-exposed Cys which could undesirably react with the fluorophore. Applicants determined that only two of the four Cys in p38α were solvent-exposed (Cys119 and Cys162) and subsequently mutated these into Ser to increase the probability that the kinase would be singly labeled only on the activation loop, which was ultimately confirmed by mass spectrometry methods (FIG. 16D to F).

### Example 13

### Crystal Structure of ac-p38α in Complex with Sorafenib

To better understand the atomic and molecular basis of the described assay principle, Applicants set out to crystallize acrylodan labeled p38α in presence and absence of Type I, Type II and Type III kinase inhibitors. So far, Applicants solved the crystal structure of ac-p38α in complex with sorafenib up to a resolution of 2.5 Å (FIG. 15). The kinase is found in its inactive state with the activation loop adopting the DFG-out conformation. Positive difference densities for the acrylodan labeled C172 and the Type II inhibitor Sorafenib are clearly visible. The fluorophore is found in an hydrophobic environment sandwiched between F168 (DFG-motif) and the P-loop. The halogenated phenyl moiety of the inhibitor occupies the allosteric binding pocket that is only present when the kinase is in its inactive conformation. Hydrogen bonding interactions between the urea moiety of the inhibitor and the side chain of E71 (helix C) and the backbone NH of D168 (DFG-loop) are clearly indicated and in accord with the previously reported b-Raf sorafenib complex (PDB-code luwh (Wan et al. Cell 2004)). The substituted pyridine binds to the hinge region of the kinase. Interestingly, the orientation of this pyridine ring is significantly different compared to the b-Raf complex. Additionally, the hinge region around M109 shows at least two conformations. At his point, Applicants cannot rule out that the observed differences in the binding mode of sorafenib and the changes in the hinge region are somehow associated with fluorophore labeling of the protein. Co-crystallization experiments of unlabeled p38α in complex with sorafenib as well as for BIRB-796 in acrylodan labeled and unlabeled p38α are underway.

### Example 14

### Application of Different Fluorophores

Applicants have recently completed initial tests of p38α labeled with a selection of three different thiol-reactive fluorophores, depicted in Table 4 which are reported to be sensitive to environmental changes. For detection of changes in the activation loop conformation, Applicants chose to examine whether these fluorophores would also be suitable for this assay approach.

The fluorophore attachment was carried out as described for acrylodan. Initial fluorescence measurements were then made to determine the optimal excitation and emission wavelengths. Real-time fluorescence measurements were then attempted using the emission maxima for each fluorophore. A single dose of 0.1 μM sorafenib was added to a cuvette containing 0.1 mM of each newly labeled p38α individually. A binding kinetic similar to that obtained under the same conditions with acrylodan-labeled p38α was obtained in all cases.

Of the new fluorophores, NBD-p38α, IAEDANS-p38α, Atto680-p38α and fluorescein-p38α had the highest sensitivity at the wavelength measured while the signal-to-noise for pyrene-p38α was poor and would likely not be suitable for this approach.

TABLE 4
Thiol-reactive fluorophores tested in the fluorescent kinase assay. The
structures of pyrene, fluorescein IAEDANS and NBD (iodoacetamide)
derivatives) are shown with acrylodan for comparison. NBD and
acrylodan are relatively small in size while pyrene and fluorescein
are considerably more bulky.

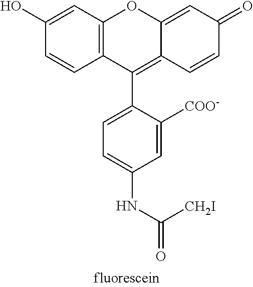

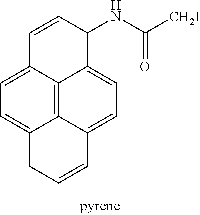

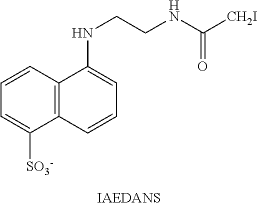

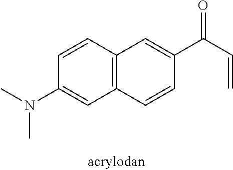

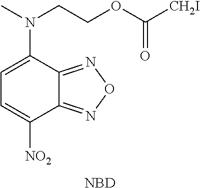

More experiments must be performed to determine the ΔI_{std} for each fluorescent-labeled p38α. Determination of ΔRₘₐₓ will not be possible since these fluorophores exhibit only changes in intensity. No shift in the emission maxima were observed for NBD, fluorescein, Atto680 or pyrene as is observed for acrylodan. Although the emission maximum does not shift to a new wavelength for IAEDANS, this fluorophore is a structural relative of acrylodan and does exhibit similar spectral behavior. Applicants were able to demonstrate reliable endpoint measurements to obtain Kd values using IAEDANS-p38α. However, NBD, fluorescein, Atto680 and pyrene primarily respond with a general increase or decrease in emission intensity without further changes in spectral shape as observed for acrylodan or less so with IAEDANS. Reliable endpoints were difficult to obtain in these cases as a result, since the inability to use ratiometric fluorescence magnifies dilution and pipetting errors between cuvettes in an endpoint titration. However, all fluorophores can be used with varying degrees of success to obtain rate constants for binding and dissociation to determine calculated Kd values. Thus, this highlights the point that the criteria for both fluorescent parameters (described in the first report) are not necessary for the development of an assay. As long as one of these criteria is met, the fluorophore-kinase conjugate has a reasonably high chance for success in this assay. However, fluorophores which permit ratiometric measurements such as acrylodan and IAEDANS are the ideal candidates for high throughput screening.

Next, the fluorescent properties of each labeled kinase were characterized by inducing the DFG-out conformation using the Type II inhibitor BIRB-796. The normalized intensity change upon saturation of p38α compared to average intensity (ΔI_{std}) and the maximal standard intensity change (ΔRₘₐₓ) between unbound and saturated DFG-out conformations of p38α were calculated for each fluorophore using the emission maxima observed in each conformational state.

According to these criteria, acrylodan-labeled p38α (ac-p38α) is confirmed to be an ideal probe for a fluorescence-based assay for detecting allosteric inhibitor binding for this kinase (see use of this probe for SAR in Table 5). Ac-p38α allows for ratiometric measurements since allosteric ligands induce a shift in the emission maximum from 468 nm to 514 nm, indicative of the movement of acrylodan from a less polar to a more polar environment (Hibbs et al., 2004; Richieri et al., 1992).

No large shifts in the emission maxima were observed for NBD, fluorescein, Atto680 or pyrene as is observed for acrylodan. However, despite a suboptimal ΔRₘₐₓ value, IAEDANS is a close structural relative of acrylodan and exhibited similar spectral behavior in response to BIRB-796 binding. Although the emission maximum of IAEDANS did not shift completely to a new wavelength, ratiometric measurements between two changing maxima (R=510 nm/465 nm) were possible. Thus, Applicants were able to obtain reliable endpoint measurements using IAEDANS-p38α which could be used to generate binding curves to directly determine K_{d} values (FIG. 29). However, it should be noted that the K_{d} for BIRB-796 was 3-fold higher when measured with IAEDANS-p38α. This is not surprising since the K_{d} values obtained with these type of approaches are often somewhat dependent on the labeling site chosen and the particular fluorophore used to carry out the measurements (de Lorimier et al, 2002).

TABLE 5
Data of thiol-reactive fluorophores tested in the fluorescent kinase assay.

| | | | | | | k_{obs} (100 nM BIRB-796) | | |
|---|---|---|---|---|---|---|---|---|
| | | | | | | Assoc. | Dissoc. | |
| Fluorophore | λ_{exc} (nm) | λ_{max, apo} (nm) | λ_{max, sat} | ΔI_{std} | ΔRₘₐₓ | (×10⁻⁴ s⁻¹) | (×10⁻⁵ s⁻¹) | K_{eq} |
| Acrylodan | 386 | 468 | 514 | 0.50 | 1.26 | 6.9 ± 2.2 | 5.1 ± 0.8 | 0.074 |
| IAEDANS | 360 | 463 | 469 | 0.80 | 0.33 | 30.3 ± 10.1 | 22.1 ± 1.6 | 0.073 |
| Fluorescein | 495 | 519 | 520 | 1.00 | 0.36 | 6.4 ± 1.8 | 16.7 ± 11.9 | 0.259 |
| Pyrene | 339 | 384 | 384 | 0.83 | 0.56 | 17.7 ± 4.9 | 37.1 ± 2.1 | 0.209 |
| NBD | 455 | 535 | 535 | 0.73 | 0.20 | 12.7 ± 4.7 | 18.4 ± 1.9 | 0.145 |
| Atto680 | 680 | 689 | 699 | 1.42 | 0.34 | 6.7 ± 2.7 | 8.3 ± 0.6 | 0.125 |

Several fluorophores were conjugated to A172C of p38α and their changing fluorescence properties were examined upon binding of BIRB-796, a known DFG-out binder of p38α.
All values for ΔRₘₐₓ and ΔI_{std} which meet the criteria deemed ideal fluorophore-protein conjugates (de Lorimier et al., 2002) appear in bold text.
The superior ΔRₘₐₓ of acrylodan is the result of a ~45 nm shift in emission maxima in the DFG-out conformation.
IAEDANS, a structural analog of acrylodan, does not exhibit a large emission shift but there is an increase in emission at ~515 nm relative to ~470 nm, allowing reliable binding curves to be measured despite the suboptimal ΔRₘₐₓ.
Pyrene and fluorescein are considerably more bulky than the other fluorophores and appear to enhance BIRB-796 dissociation rates, resulting in higher calculated equilibrium constants (K_{eq}) for 100 nM BIRB-796 under these experimental conditions.
[Note: The chemical structure of Atto680 has not been released by the manufacturer (http://www.innovabiosciences.com).]

In the case of NBD, fluorescein, Atto680 and pyrene, binding of BIRB-796 to the DFG-out conformation of p38α caused a general decrease in emission intensity at a single wavelength without any accompanying changes in spectral shape (FIG. 30). Since all tested fluorophores met the criteria for ΔI_{std}, they could be used successfully to obtain rate constants for binding and dissociation. Such kinetic information can ultimately be used to calculate the K_{d} of ligands without directly measuring binding curves. Thus, it is not necessary that fluorescent-tagged kinases labeled on the activation loop meet the criteria for both ΔI_{std} and ΔRₘₐₓ in order to provide useful information. As long as one of these criteria is met, the labeled kinase has a reasonably high chance of successfully detecting allosteric inhibitor binding and changes in the activation loop conformation. However, fluorophores which permit ratiometric measurements such as acrylodan and IAEDANS are the ideal candidates for directly determining the K_{d} of a ligand and will have the highest chance for success and reliability when implemented into higher cost HTS platforms.

### Example 15

### Biological Methods

Fluorescent Labeling of Chicken cSRC & Development of a Novel Screening Assay

Crystal structures of chicken cSrc (kinase domain) in the DFG-in and DFG-out conformations were closely examined to identify a suitable fluorophore attachment site near the N-terminal end of the activation loop of cSrc which would report the binding of allosteric inhibitors by sensing changes in the activation loop conformation. Care was taken to not choose residues that were known phosphorylation sites or other sites that appeared to be critical to maintaining protein stability. A Cys was introduced into the chosen position (L407C) by site-directed mutagenesis while non-specific labeling was minimized by conservatively mutating other solvent exposed Cys into Ser (C277S, C483S, C496S).

Due to its relatively small size, high sensitivity to polarity changes and well-documented use in the formation of biosensor conjugates (de Lorimier et al., 2002), acrylodan (thiol-reactive) was preferred for the labelling of the activation loop of the kinase. Pure cSrc kinase containing the labeling site Cys mutation (L407C) and acrylodan (dissolved in DMF) were combined in buffer (pH 7.0) at a ratio of 1:1.5 protein:fluorophore and allowed to react in the dark overnight at 4° C. The amount of DMF present during conjugation did not exceed 0.1% v/v. The conjugated cSrc was then concentrated and washed 3 times with Measurement Buffer (50 mM Hepes, 200 mM NaCl, pH 7.45) to remove unreacted fluorophore. The labeled cSrc was then aliquoted, kept dark and frozen at −20° C. Labeling was subsequently verified by mass spectrometry analysis of trypsinized fragments of the labeled and unlabeled proteins (FIG. 16). Fluorescence characterization of cSrc with inhibitors which bind to the DFG-in (dasatinib) and DFG-out conformations are shown in FIG. 17.

In Vitro Kinase Activity Assay for cSrc Variants

A biotinylated poly Glu-Tyr substrate peptide was phosphorylated by cSrc. After completion of the reaction, an anti-phosphotyrosine antibody labeled with Europium Cryptate and Streptavidin labeled with the fluorophore XL665 were added. The FRET between Europium Cryptate and XL665 was measured to quantify the phosphorylation of the substrate peptide. ATP concentrations were set at their respective Km values (15 μM for the wild type cSrc and 1 μM for cSrc-T338M) and 100 nM of substrate were used for both wild type and drug resistant cSrc. Kinase, substrate peptide and inhibitor were pre-incubated for 2 hours before the reaction was started by addition of ATP. IC₅₀ determinations for cSrc kinase were measured with the HTRF® KinEASE TM-TK assay from Cisbio (Bagnols-sur-Ceze, France) according to the manufacturer's instructions. A Tecan Safire² plate reader was used to measure the fluorescence of the samples at 620 nm (Eu-labeled antibody) and 665 nm (XL665 labeled Streptavidin) 60 μs after excitation at 317 nm. The quotient of both intensities for reactions made with 8 different inhibitor concentrations was fit to a Hill 4-parameter equation to determine IC₅₀ values. Each reaction was performed in duplicate and at least three independent determinations of each IC₅₀ were made.

Analysis of cSrc Labeling by HPLC and Mass Spectrometry

Proteins were trypsinized according to standard procedures prior to HPLC and mass spectrometry analysis to confirm the conjugation of the fluorophore to the desired protein fragment. Unlabeled and labeled cSrc (60 μg) were incubated separately with proteomics grade trypsin (3 μg) in 55 mM NH₄CO₃ with 10% v/v acetonitrile. Samples were incubated overnight at 37° C., frozen in liquid nitrogen, and lyophilized. The lyophilized powder was then resuspended in 75 μl of water for analysis. Digested peptide fragments were then separated and purified using an HPLC (Agilent 1100 Series) equipped with a binary pump, thermostated auto sampler and diode array detector. Samples were passed through a Waters (Milford, Mass., USA) Atlantis dC18 column (2.1 mm×150 mm) with 3 μm particle size at ambient temperature. Samples were run at 0.2 ml/min with the following gradient: 100% Solvent A (0.1% formic acid in water) for 5 min, ramping up to 60% Solvent B (0.1% formic in acetonitrile) with a linear gradient in 55 min, then increasing to 80% Solvent B in 10 min before holding at 80% Solvent B until 90 min. The mass spectrometer (Thermo LTQ) was equipped with a standard electrospray ion source (source voltage=4 kV). An automatic MS/MS analysis was performed for the most intense peaks (minimal signal intensity of 10,000 required) in a triple play experiment (normal MS, zoom scan of the most intense peaks, followed by MS/MS in the case where the charge state was 2 or higher.) 35% normalized collision energy was used for MS/MS analysis.

### Allosteric Inhibitor Screen and Kd Determination

Screening initiatives were carried out for acrylodan-labeled cSrc in 384-well plates. Stocks of candidate compounds were prepared in DMSO at 20× the final desired concentration. Compounds were mixed with labeled cSrc in triplicate at final concentrations of 10 and 50 μM. Each well contained 1 μl of compound+19 μl of Measurement Buffer (+0.01% v/v Brij-35) containing 100 nM kinase (5% v/v DMSO after mixing). Plates were covered with an adhesive aluminum foil and incubated for 15-30 min at RT prior to measurement of emission intensities at 445, 475 and 505 nm using a Tecan Safire2 plate reader. Acrylodan was excited at 386 nm. Binding was measured using the ratio of λ445/λ475 (FIG. 18a) while inhibitor binding mode was revealed by the ratio of λ505/λ475 (FIG. 18b). Additional details on the fluorescence characterization are provided in FIG. 17. Potential hits were subjected to further titration studies in cuvettes or 96-well plates to obtain K_{d} values.

### Cell Culture

PC3 and DU145 were generously provided by Dr. Roman Thomas (Max Planck Institute for Neurological Research, Cologne). The cells were cultured in Dulbecco's modified Eagle's medium (DMEM) supplemented with 10% heat-inactivated fetal bovine serum (FBS) and 100 units/mL penicillin/streptomycin. Cells were cultured at 37° C. in humidified air containing 5% CO2. After inhibitor treatment (5 h), the cells were washed twice in cold phosphate-buffered saline (PBS) and then lysed for 10 min on ice in lysis buffer (20 mM Tris-HCl pH 7.5, 150 mM NaCl, 1% Triton, 1 mM Na₂EDTA, 1 mM EGTA, 2.5 mM sodium pyrophosphate, 1 mM β-glycerophosphate, 1 mM Na₃VO₄, 1 μg/mL leupeptin, 1 mM PMSF, and common protease inhibitors). Subsequently, cells were centrifuged for 20 min at 20000×g and 4° C. The supernatant was subjected to immunoblot analysis.

### Immunoblot Analysis of Src and FAK

Protein concentration was measured using a spectrophotometer (ND-1000, peQLab). Equal amounts of protein were separated by SDS-PAGE and transferred to nitrocellulose membranes. Blots were blocked for one hour in Tris-Buffered Saline with Tween-20 (TBST) supplemented with 5% non-fat milk and subsequently incubated over night at 4° C. in primary antibody, namely anti-phospho-FAK, anti-phospho-Src, anti-FAK, and anti-Src. All antibodies were obtained from Cell Signaling Technology. After washing, blots were incubated with secondary antibodies and then detected on film using the enhanced chemiluminescence (ECL) detection system.

### Example 16

### Chemical Synthesis

The synthesis protocols of quinazolines are well known in the art. Protocols for the synthesis of pyrazoloureas are described e.g., in Regan et al. (2003) and in other publications referred to within this application.

### Example 17

### Crystallization and Structure Determination

Crystallization and Data Collection of cSrc-RL37, cSrc-RL38, cSrc-RL45 and cSrc-T338M-RL45

For the cSrc-RL37 and RL38 complex structures, Applicants obtained crystals by the hanging drop vapour diffusion method by pre-incubating inhibitor (prepared in DMSO) with kinase (stored in 20 mM Tris pH 8.0, 100 mM NaCl, 1 mM DTT) to form the enzyme-inhibitor complex prior to crystallization. In the case of RL37 and RL38 500 μM inhibitor was pre-incubated with 330 μM wild type chicken cSrc for 2 hr. Crystals were grown at 20° C. after mixing 1 μL protein-inhibitor solution with 1 μL reservoir solution (0.1 mM MES (pH 6.9), 4% glycerol, 10% PEG 4000 and 50 mM sodium acetate) (Seeliger et al., 2007). Plate shaped crystals of the tri-clinic space group P1 grew within one day. In case of RL45, the same concentration of inhibitor was pre-incubated along with 180 μM wild type cSrc or cSrc-T338M for 4 hr. Crystals were grown using the sitting drop method at (20° C.) after mixing 0.2 μL protein-inhibitor complex and 0.2 μL reservoir solution (85 mM MES (pH 6.5), 10.2% PEG 20000, 15% (v/v) glycerol). Drops were pipetted using a Mosquito Nanodrop crystallization robot (TTP LabTech Ltd., Melbourn, UK). For the crystals of cSrc with inhibitors RL37 and RL38 20% glycerol was used as cryo protectant before they were flash frozen in liquid nitrogen. Crystals of cSrc with RL45 were directly frozen without the addition of glycerol.

Diffraction data of all cSrc-inhibitor complex crystals were collected at the PX10SA beamline of the Swiss Light Source (PSI, Villingen, Switzerland) to a resolution of 2.5 Å for cSrc-RL37 and cSrc-RL38 and 2.6 Å for cSrc-RL45, using wavelengths close to 1 Å. The datasets were processed with XDS (Kabsch, 1993) and scaled using XSCALE (Kabsch, 1993).

Structure Determination and Refinement of cSrc-RL37, cSrc-RL38, cSrc-RL45 and cSrc-T338M-RL45

All four cSrc-inhibitor complex structures were solved by molecular replacement with PHASER (Read, 2001) using the published cSrc structure 2OIQ (Seeliger et al., 2007) as template. The two cSrc molecules in the asymmetric unit were manually modified using the program COOT (Emsley and Cowtan, 2004). The model was first refined with CNS (Brunger et al., 1998) using simulated annealing to remove model bias. The final refinement was performed with REFMAC5 (Murshudov et al., 1997). Inhibitor topology files where generated using the Dundee PRODRG2 server (Schuttelkopf et al., 2004). Refined structures were validated with PROCHECK (Laskowski et al., 1993).

### Accession Codes

Coordinates and structure factors have been deposited under the following accession codes to the Protein Data Bank: cSrc bound to RL37, 3F3U; cSrc bound to RL38, 3F3T; cSrc bound to RL45, 3F3V and cSrc-T338M bound to RL45, 3F3W.

### Example 18

### Identification of Type III Inhibitors for cSrc Kinase

Since it has been proposed to be overexpressed or upregulated in several tumors types—notably in gastrointestinal and prostate cancer (Yeatman, 2004)—and no Type III inhibitors have yet been reported, Applicants selected the tyrosine kinase cSrc. Additionally, the gatekeeper in cSrc was predicted to be a hotspot for drug resistance mutations against ATP competitive inhibitors even before the first clinical incidences for EGFR and Abl kinase were reported Blencke et al., 2003). Numerous crystal structures of cSrc in the DFG-in (Breitenlechner et al., 2005) and DFG-out (Dar et al., 2008, Seeliger et al., 2007) conformation are available in the Protein Data Bank, suggesting that Applicants' assay would succeed with this kinase. In a screening initiative, Applicants employed Applicants' newly developed fluorescent-tagged cSrc assay to identify four pyrazolourea compounds (3-6) (also designated 1a-1d in the associated figure) as Type III allosteric binders to cSrc with K_{d} values in the 1 μM range (FIG. 19). Although the binding of Type III inhibitors has not yet been reported for cSrc kinase, several pyrazoloureas are known to be potent Type III binders of p38α kinase with affinities in the low nM range (Pargellis et al., 2002; Dumas et al., 2000) and form the core scaffold from which the mentioned Type II p38α inhibitor BIRB-796 was developed. While binding of (3-6) was detected using the fluorescent cSrc, an accurate determination of the K_{d} was not possible due to limited compound solubility above 50 μM. Enzyme activity assays were subsequently used to confirm that these screening hits indeed inhibit cSrc kinase activity, and again due to limited solubility, Applicants were only able to observe inhibition of cSrc by (3, RL57) and (5, RL38) which appear to have IC₅₀ values also in the mid 1 μM range (FIG. 19b). Considering the shared R2 aniline moiety in (3-5), the preference for (3, RL57) in both assay formats suggests that the size and degree of hydrophobicity of the R1 aryl substituents may be an important determinant for more energetically favorable binding to inactive cSrc. The same activity assays were then carried out using the drug resistant cSrc variant (T338M) (Blencke et al., 2003; Michalczyk et al., 2008) and revealed that the presence of a bulkier gatekeeper residue had no effect on (3, RL57) activity when compared to wild type cSrc while (5, RL38) appeared to no longer be active, further highlighting the importance of the R1 moiety of (3, RL57) in contributing to its affinity to cSrc. Unlike Type I inhibitors such as quinazolines (9, RL56), (10, RL6) (also called 2b and 2c, respectively, in the associated figure) and the aminothiazole dasatinib, which show a dramatic loss in potency in cSrc-T338M (FIG. 2b), this residue is not expected to interfere with compounds that have the optimal size and degree of hydrophobicity to bind behind the gatekeeper position and exclusively within the allosteric pocket.

### Example 19

### Complex Structure of a Type III Inhibitor in cSrc

To better understand the affinity and selectivity profile of these compounds and to confirm binding to the allosteric site of the inactive kinase (DFG-out conformation) Applicants crystallized several pyrazoloureas in complex with cSrc and obtained high quality diffracting crystals for the (4, RL37) (FIG. 20) and (5, RL38) cSrc complexes. The structures were solved in space group P1 by molecular replacement with two molecules in the asymmetric unit and the coordinates of both structures refined to 2.5 Å. The activation loop and adjacent helix C of the kinase are found in the DFG-out conformation and the inhibitor is well defined by its electron density and resides in the expected allosteric site of the kinase domain (FIG. 20a). To Applicants' knowledge, this is the first reported crystal structure of a non-receptor tyrosine kinase in complex with a Type III inhibitor. Each of the two protein molecules superimpose well with the inactive cSrc-imatinib structure (Seeliger at al., 2007). Analogous to this published structure, F405 of the DFG motif was displaced by the inhibitor and flipped into the ATP binding site to adopt the DFG-out, or inactive conformation, rendering it inaccessible to binding of ATP. Additionally, critical hydrogen bonding interactions between the DFG-loop and helix C of the kinase domain and the urea moiety of the inhibitor are conserved and isostructural to what has been reported for other urea derivatives in complex with cSrc (Dar et al., 2008), B-Raf (Wan et al., 2004) and p38α (Pargellis et al., 2002).

### Example 20

### Design of Potent Type II Hybrid Inhibitors for cSrc Kinase

Given the moderate μM IC₅₀ values of these Type III pyrazoloureas in cSrc, Applicants set out to use the X-ray crystal structures obtained here and of quinazolines in complex with cSrc published previously (Michalczyk et al., 2008) to design larger inhibitor molecules with an increased affinity to cSrc. Applicants superimposed the cSrc-RL37 complex with one of Applicants' recently solved cSrc structures in complex with a 4-amino-quinazoline (Michalczyk et al., 2008) and found that the phenyl substituents of both inhibitor scaffolds (4-aminophenyl of the quinazoline and R1 of the pyrazolourea) nicely align near the Thr338 gatekeeper side chain (FIG. 20b), suggesting that a more potent inhibitor could be generated by fusing both scaffolds via a 1,4-para or 1,3-meta-substituted linker moiety (FIG. 20c). Applicants were stimulated by both, fragment based design approaches—where molecule fragments identified by NMR (Shuker et al., 1996) or protein X-ray crystallography (Gill et al., 2005; Nienaber et al., 2000) can be efficiently linked or grown to generate molecules with increased affinity—and by the emerging concepts of the rational design of DFG-out binders (Liu and Gray, 2006; Jacobs et al., 2008). Both methods have been proven to be powerful in kinase lead discovery projects (Okram et al., 2006; Warner et al., 2006).

Since pyrazoloureas have proven to be privileged motifs for the inhibition of p38β and bind behind the gatekeeper residue, a position frequently associated with drug resistance in kinases, Applicants also wanted to use these allosteric scaffolds as starting points to study determinants for kinase inhibitor selectivity for further structure-guided design processes which take into account larger gatekeeper side chains. Applicants docked the proposed 1,4-para and 1,3-meta hybrid compounds into a published structure of BIRB-796 bound to the DFG-out conformation of p38α (Pargellis et al., 2002) and observed different binding site geometries in the vicinity of the gatekeeper residue and the DFG-motif when compared to inactive cSrc. Given these observations, Applicants predicted that cSrc would better accommodate hybrid compounds fused via a 1,4-para linkage while a 1,3-meta linkage should favor binding to p38α. More importantly, Applicants predicted that the 1,4-substitution pattern in these compounds would provide the optimal geometry to avoid steric clashes with larger amino acid side chains at the gatekeeper position as found in drug resistant kinases. Although the 4-aminoquinazolines (9, 10) and the identified pyrazoloureas (3 and 5) are alone weak inhibitor fragments with IC₅₀s in the μM range in wild type cSrc, Applicants expected that the resulting 1,4-substituted hybrid compounds would not only show significantly increased potency in inhibiting wild type but also the otherwise drug resistant cSrc-T338M mutant variant.

### Example 21

### Synthesis of a Focused Library of 4-amino-pyrazolourea-quinazolines as Novel Type II Inhibitors

Applicants synthesized a small focused library of fused quinazoline pyrazoloureas as novel inhibitors of cSrc (FIG. 21 and FIG. 22). The panel included analogs with varying inhibitor geometries designed to orient around the steric gatekeeper residue of drug resistant cSrc-T338M or to preferentially bind to p38α, a kinase which is known to be inhibited potently by compounds containing these types of pyrazolourea scaffolds.

### Example 22

### In Vitro Characterization of Novel Type II Hybrid cSrc Inhibitors

To test whether the allosteric site in cSrc confirmed by Applicants' co-crystallization experiments is indeed druggable in solution and to test the above mentioned hypotheses regarding inhibitor selectivity to p38α and drug resistant cSrc-T338M, Applicants first measured the K_{D} of each compound using the fluorescent-labeled kinase assay system described above. The binding data obtained from each fluorescent kinase confirmed the expected binding preference of 1,4- and 1,3-substituted hybrid compounds for cSrc and p38α, respectively. Additionally, Applicants performed enzyme activity assays for cSrc (wild type and drug resistant) using several Type II hybrid compounds (FIG. 21) to confirm inhibition of phosphotransfer. Applicants observed a significant (up to 4 orders of magnitude) increase in potency in the measured IC50 values of these compounds when compared to the pyrazolourea (3, 5) or quinazoline (9, 10) moieties that were used to construct each Type II hybrid. The measured K_{D} values are slightly higher when compared to the IC₅₀ values, but follow the same trends for 1,4- and 1,3-substituted hybrid compounds. Lastly and most importantly, the kinetics clearly demonstrate that the 1,4-substituted hybrid compounds (11a-c) (also called 3a-3c in the associated figure) show no loss of potency in the cSrc-T338M mutant in vitro.

### Example 23

### Crystallisation of p38 with Inhibitors

Various inhibitors were co-crystallized with wild type p38α using conditions similar to those previously reported for unmodified p38α (Bukhtiyarova et al., 2004). Briefly, protein-inhibitor complexes were prepared by mixing 30 μL p38α (10 mg/mL) with 0.3 μL of inhibitor (100 mM in DMSO) and incubating the mixture for 1-2 hrs on ice. Samples were centrifuged at 13,000 rpm for 5 min to remove excess inhibitor. Crystals were grown in 24-well crystallization plates using the hanging drop vapor diffusion method and by mixing 1.5 μL protein-inhibitor solution with 0.5 μL reservoir (100 mM MES pH 5.6-6.2, 20-30% PEG4000 and 50 mM n-octyl-(3-D-glucopyranoside).

For the crystals of p38α with inhibitors 20% glycerol was used as cryo protectant before they were flash frozen in liquid nitrogen. Diffraction data of the p38α-SB203580 and p38α-RL45 complex crystals were collected at the PX10SA beamline of the Swiss Light Source (PSI, Villingen, Switzerland) using wavelengths close to 1 Å. Diffraction data of the p38α-RL48, p38α-RL62 and p38α-sorafenib complexes were collected in-house. All data sets were processed with XDS (Kabsch, 1993) and scaled using XSCALE (Kabsch, 1993). All p38α-inhibitor complex structures were solved by molecular replacement with PHASER (Read, 2001) using the published p38α structures (PDB code: 1ZYJ) (Michelotti et al., 2005) or (PDB-code: 2EWA) (Vogtherr et al., 2006) as templates. The molecules in the asymmetric unit were manually modified using the program COOT (Emslex and Cowtan, 2004). The model was first refined with CNS (Brunger et al., 1998) using simulated annealing to reduce model bias. The final refinement was performed with REFMAC5 (Murchudow et al., 1997). Inhibitor topology files where generated using the Dundee PRODRG2 server (Schuttelkopf and van Aalten, 2004). Refined structures were validated with PROCHECK (Laskowski et al., 1993). PyMOL (de Lano, 2002; http:///www.pymol.org) was used to produce the figures.

### Example 24

### Complex Crystal Structures of Novel Type II Inhibitors in cSrc and Drug Resistant cSrc-T338M Mutant Variant

To get deeper insights into the binding mode of this class of Type II inhibitors and to understand how these 1,4-substituted inhibitors can bypass a bulky Met gatekeeper residue, Applicants cocrystallized cSrc (wild type and drug resistant) with RL45 (11b) and found that the compound binds to the DFG-out conformation and adopts the proposed Type II inhibitor binding mode which spans from the allosteric site into the distal ATP binding pocket (FIG. 23). Briefly, N1 of the quinazoline moiety makes direct hydrogen bonding interactions with the hinge region (M341) of the kinase, which is typically observed for quinazoline binding to cSrc (Michalczyk et al., 2008), CDK2 (Shewchuk et al., 2000), p38α (Shewchuk et al., 2000), Aurora (Heron et al., 2006) and EGFR (Blair et al., 29′007; Stamos et al., 2002). The pyrazolourea moiety resides in the allosteric site formed by helix C and the N-terminal region of the activation loop and forms identical hydrogen bonding interactions with the protein as seen for the cSrc-RL37 and cSrc-RL38 complexes. The central phenyl ring of the inhibitor that bridges the quinazoline and pyrazolourea scaffolds is sandwiched between the gatekeeper residue and the F405 of the DFG motif. Interestingly, in the cSrc-T338M-RL45 complex the presence of the sterically demanding Met gatekeeper forces the central phenyl moiety of the inhibitor to flip by 90° to avoid the steric clash with the amino acid side chain such that the plane of the phenyl ring of the inhibitor now faces Cε of M338. Additionally, the side chain of F405 rotates by 90° to conserve the electrostatically favourable edge-to-face orientation Hunter et al., 1991) of both π-electron systems (inhibitor phenyl and phenyl side chain of F405) (FIGS. 23c and 23d). Rotation of the central phenyl element in 1,3-substituted hybrid compounds (11d and 11e) (also called 3d and 3e in the associated figure) is not possible without disrupting binding of either the quinazoline or pyrazolourea moiety with the protein and provides an explanation why 1,3-disubstituted hybrids such as (11d) and (11e) do not bind to drug resistant cSrc-T338M (FIG. 24).

### Example 25

### Type II cSrc Inhibitors Disrupt Cell-to-Cell Contacts in cSrc-Dependant Cancer Cell Lines

To assess cSrc inhibition by RL46 (11c) in cellular systems, Applicants treated PC3 and DU145 prostate carcinoma cell lines with different concentrations of RL46 (11c), 100 nM dasatinib (positive Src inhibition control), or vehicle (DMSO). Applicants monitored the phosphorylation state of Y416—an autophosphorylation site in the activation loop of cSrc—and Y576/Y577—two residues in the activation loop of focal adhesion kinase (FAK). FAK is a non-receptor tyrosine kinase substrate of cSrc which localizes to focal adhesions that form between cells and is a key regulator of cell cycle progression, cell survival and cell migration (Schaller, 2001). The phosphorylation and activation of FAK on Y576 and Y577 by cSrc kinase is required for the full enzymatic activity of FAK, causing the disruption of focal adhesions, resulting in loss of cell-cell and cell-matrix contacts and apoptosis (Yeatman, 2004; Calalb et al., 1995). The overexpression of FAK and cSrc has been shown to lead to increased cell invasion and metastasis in both breast and colon cancers (Novakowski et al., 2002). Following 5 hr treatment of confluent PC3 and DU145 cells with dasatinib or RL46 (11c), pSrc and pFAK levels were markedly reduced (FIG. 25a). This correlated with distinct change in cellular phenotype, exhibited by loss of cell adhesion and a significant reduction in the number of cells (FIG. 25b). Applicants' results clearly demonstrate that the observed phenotype changes are due to the direct inhibition of cSrc kinase by Applicants' hybrid compound in these two cancer cell lines.

### Example 26

### Kinase Selectivity Profile of Direct Inhibitor Binding

In order to determine kinase selectivity for Applicants' newly developed Type II hybrid compounds, kinase profiling was performed for RL45 (11b) against a selected subpanel of 64 different kinases at a concentration of 5 μM (Ambit Biosciences) (FIG. 26). The inhibitor profile shows a tendency for RL45 (11b) to bind to phylogenectically distinct kinases that can adopt the DFG-out conformation with a distinct preference for two major kinase groups: (i) TK (tyrosine kinase family) and (ii) CMGC (serine-threonine kinases in the CDK, MAPK, GSK3 and CLK families). It is interesting to note that the profiling RL45 (11b) revealed a strong preference for binding tightly to most (but not all) TKs. Although the binding of RL45 (11b) to numerous serine/threonine kinase families (i.e. CAMK and AGC families) was scored as very poor in most cases, RL45 (11b) showed a distinct preference for the CMGC family of serine-threonine kinases. Analysis of the sequence alignments of these kinases reveals that most of these CMGC kinases contain a Phe or Thr gatekeeper. Applicants have clearly shown the structural details of how RL45 (11b) can overcome these large gatekeepers in TKs such as mutant cSrc and the formation of a favorable edge-to-face π-π interaction between the central phenyl of the inhibitor and the Phe of the DFG motif. Applicants predict that the Phe gatekeeper observed in several of the tested CMGC family of serine-threonine kinases may also stabilize the inhibitor by a similar mechanism, resulting in the potent inhibition of these kinases by RL45 (11b). TK were also sensitive to RL45 (11b) and Applicants attribute this to the smaller gatekeepers (Thr or Val) found in most TKs. The combination of in vitro binding and activity assays demonstrates that the quinazolinepyrazolourea hybrids presented here are promising kinase inhibitor scaffolds for further medicinal chemistry initiatives to direct inhibitor selectivity. The gatekeeper is a Thr in many tyrosine kinases and also serves as a crucial determinant of Type I inhibitor selectivity and affinity. Therefore, the development of these Type II hybrid inhibitors combined with the observation of a potential cross-talk between the inhibitor and the side chains of the drug resistant hydrophobic gatekeeper and the DFG phenylalanine residue provides an attractive chemical biological strategy for overcoming the increasingly common gatekeeper mutationassociated drug resistance.

### Example 27

### SAR of type II and type III Inhibitors on p38

Applicants designed and generated a focused library of pyrazoloureas, a class of compounds whose pharmacophore and binding mode are known in p38α and used several new derivatives of this scaffold to examine structure-activity relationships (SAR) and characterize the fluorescence response of ac-p38α. Pyrazoloureas represent one of the prototypes for Type III and Type II kinase inhibitors. Type III pyrazoloureas not only stimulated the development of the former clinical candidate BIRB-796 (Regan et al., 2002) but also provided a wealth of structural and kinetic data, allowing for comparison of K_{d} values determined here using ac-p38α (FIG. 31) with other approaches (Pargellis et al., 2002; Regan et al., 2003; Kroe et al., 2003). Several of these known compounds were also synthesized to serve as measuring stick for Applicants' fluorescent-tagged kinase binding assay.

As expected, the K_{d} of BIRB-796 was time-dependent (FIG. 9) as reported elsewhere (Pargellis et al., 2002). All Type III inhibitors also showed this time-dependence, but required less time (2-4 hr) to reach equilibrium with p38α. Thus, long pre-incubation times are necessary to assure complete binding of Type II and Type III ligands before making endpoint measurements. In general, Applicants found excellent agreement between Applicants' K_{d} values and those reported elsewhere for known compounds using other approaches, with the largest differences occurring for compounds with a published K_{d} of <10 nM. As discussed above for BIRB-796, the affinity of DFG-out binders to p38α is dictated primarily by k_{off}. Therefore, Applicants believe that the calculated K_{d} values reported in the literature for the strongest binders are more subject to the experimental conditions under which k_{off} was measured and have been shown to vary significantly depending on the methods used (Kroe et al., 2003). However, certain trends in the SAR are always maintained with respect to aryl moiety I (substituted phenyl attached to the urea) and aryl moiety II (substituted phenyl attached to the pyrazole) regardless of the assay system used to make the measurements.

In the case of aryl moiety I, Applicants observed a noticeable affinity ranking such that 13 (RL33)(phenyl)<<12e (RL34) (4-chlorophenyl)<6 (RL35) (naphthyl), a SAR trend which is well-documented (Pargellis et al., 2002; Regan et al., 2002; Regan et al., 2003; Kroe et al., 2003; Regan et al., 2003). Moiety I fits into a small hydrophobic sub-pocket behind the gatekeeper residue of p38α and the bulkier naphthyl moiety forms better lipophilic interactions as a result of its ability to penetrate deeper into this sub-pocket. It could also be argued that more solvation entropy is gained (release of more water molecules) upon burial of the bulkier naphthyl in this sub-pocket (Lafont et al., 2007). Thus, a phenyl moiety alone at this position does not contribute significantly to the affinity of the compound, explaining the much higher K_{d} of 13 (RL33) in comparison to 12e (RL34) and 6 (RL35). This is further confirmed by 15, RL39, which has a central phenyl in place of the bulkier naphthyl of BIRB-796, resulting in a 50-fold higher K_{d}. Similar findings for these compounds were also observed elsewhere using other approaches (Regan et al., 2002). Given the hydrophobic characteristics of this sub-pocket, it is also not surprising to see a significant loss of affinity for the more polar 4-aminophenyl 16a,b (RL43, RL42) or 3-aminophenyl 17a, b (RL41, RL79) Type III derivatives.

The addition of a mono-substituted aryl moiety II to the N1 of the pyrazole of 1, RL8 extends the ligand into the allosteric pocket and results in a significant increase in the affinity. This phenyl ring shows a distinct preference for substituents in the para and meta positions in Applicants' assay, a SAR trend which is also well-documented (Regan et al., 2002). To further investigate the importance of the substitution pattern of moiety II, Applicants used the crystal structure of p38α in complex with BIRB-796 (PDB-code: 1kv2) together with in silico modeling to design and synthesize a bulkier inhibitor 7 (RL19). Applicants hypothesized that the combination of a 4-trifluoromethyl and 2,6-dichloro substitution pattern on the phenyl ring would prevent the compound from fitting into the allosteric pocket, which was confirmed by the complete lack of fluorescence response from ac-p38α.

Above in Example 20, Applicants reported the development of several Type II quinazoline-pyrazolourea hybrid inhibitors of cSrc kinase. In the case of cSrc, smaller Type III pyrazoloureas were found to inhibit cSrc with mid μM IC₅₀ values. In a manner similar to the development of BIRB-796 from 1, RL8 (Regan 2002), the fusion of these compounds with a quinazoline scaffold, which are also μM inhibitors of cSrc (Michalczyk et al., 2008), resulted in potent low nM Type II inhibitors that extend into the ATP binding site to interact with the hinge region. Furthermore, Applicants showed that the 1,4-para fused hybrids 11a (RL44), 11b (RL45) and 11c (RL46) were better binders to cSrc while using molecular modeling to predict that p38α would better accommodate the analogous 1,3-meta fused inhibitors 11d (RL61) and 11e (RL62). Using ac-p38α, Applicants were able to confirm this hypothesis and found that 11d (RL61) and 11e (RL62) have a 6-fold higher affinity over 11a (RL44) and 11b (RL45). Applicants solved the crystal structure of 11e (RL62) and 11b (RL45) each in complex with p38α and observed additional stabilizing interactions which explain why 1,3-meta hybrids have a higher affinity for p38α (FIG. 32). Applicants also performed an extensive analysis of the kinetic rate constants for these compounds using ac-p38α (Table 6) and found that the dissociation rate of 11e (RL62) is slower than that of 11b (RL45), while both compounds exhibit similar k_{obs} for binding (1.40±0.25×10⁻³ s⁻¹ (n=3) for 11e (RL62) and 1.36±0.13×10⁻³ s⁻¹ (n=3) for 11b (RL45), respectively) which may partially account for the higher affinity of 11e (RL62). Lastly, these detailed kinetic and structural characterizations of 1,3- and 1,4-fused hybrid compounds led us to design and synthesize several pyrazolourea-quinoline analogues as novel compounds that are potent Type II inhibitors of p38α.

TABLE 6
Dissociation of several DFG-out binders from ac-p38α

| | central | substitution | k_{off} × 10⁻⁵ | t_{1/2} |
|---|---|---|---|---|
| | ring moiety | pattern* | (s⁻¹)* | (min)* |
| BIRB-796 | naphthyl | 1,4-para | 5.1 ± 0.5 | 226.5 |
| 12a (RL29) | p-Cl-phenyl | 1,4-para | 22.1 ± 05.8 | 52.3 |
| 15 (RL39) | phenyl | 1,4-para | 102.8 ± 13.0 | 11.2 |
| 11e (RL62) | phenyl | 1,3-meta | 115.9 ± 11.9 | 10.0 |
| 11b (RL45) | phenyl | 1,4-para | 169.5 ± 4.6 | 6.8 |
| sorafenib | phenyl | 1,4-para | 482.7 ± 133.8 | 2.4 |

*Kinetic parameters (t_{1/2} of dissociation and k_{off}) were determined for the dissociation of several Type II/III compounds from acrylodan-labeled p38α. All measurements were made a minimum of 3 times.
[Note:
All binding and dissociation curves were fit to a single exponential equation: F(t) = F(∞) + F(0) exp(−t*k_{obs}), where t is time, F(0) is the initial fluorescence intensity and F(∞) is the fluorescence at t = ∞. The half time (t_{1/2}) was calculated with the following equation: t_{1/2} = ln 2/k_{obs}.]

### SAR of Additional Type II Hybrid Inhibitors

To better understand the preference of p38α for 1,3-meta hybrids, Applicants solved the crystal structure of both 11e (RL62) and 11b (RL45) in complex with the wild type kinase (FIG. 32). Both compounds cause the activation loop to adopt the DFG-out conformation and each inhibitor binds with a Type II binding mode. However, distinct differences in the structural rearrangement of the DFG motif were observed which explained the preference for the 1,3-meta hybrid 11e (RL62) in p38α. More specifically, Phe169 of the DFG motif moves by ˜4 Å to a position next to the plane of the quinazoline core, resulting in the formation of a favorable edge-to-face orientation of both π-electron systems. Additionally, there is an intricate water-mediated hydrogen bonding network formed between the inhibitor and the DFG motif which is not observed in the RL45-p38α complex. These additional stabilizing effects on the DFG-out conformation may explain the higher affinity of the 1,3-meta hybrids for p38α.

Interestingly, the N3 of the quinazoline seems to be within hydrogen bonding distance to the side chain of the Thr106 gatekeeper in both complexes with p38α. To further investigate the role of this interaction, Applicants investigated the potency of a 1,4-para quinoline hybrid 11c (RL46) but found no significant loss in affinity compared to its quinazoline hybrid counterpart 11b (RL45), suggesting that this interaction may not be essential for the binding of this series of compounds.

Applicants furthered the SAR of the hybrids 11a-h by generating analogous compounds 11i-p where the methyl substituent on the phenyl extending from N1 of the pyrazole (moiety II) is moved from the meta (3-methyl phenyl) to the para (4-methyl phenyl) position. Applicants found that this small change resulted in a slight increase and decrease in affinities of 1,4-para hybrids and 1,3-meta hybrids, respectively, such that their K_{d} values are no longer significantly different in p38α. Applicants observed a 2-fold loss of affinity for the 1,3-meta quinoline hybrid 11n (RL51) in comparison to its quinazoline analog 11l (RL48). Unlike in the tyrosine kinase cSrc, it is important to note that these 1,4-para Type II hybrid compounds are worse inhibitors in p38α when compared to their smaller Type III pyrazolourea counterparts, making them less optimal inhibitors of p38α.

Applicants could postulate that increased movements of the C helix in tyrosine kinases such as cSrc (Levonson et al., 2006) may allow for these larger hybrid molecules more room to sample the binding site and find a better fit. Movements of the C helix—which forms a significant part of the “roof” of the allosteric pocket—may also explain the significantly reduced affinity of Type III scaffolds in cSrc in comparison to p38α. The C helix of p38α does not sample multiple conformations, thus resulting in a more rigid pocket and more thermodynamically favorable binding of Type III pyrazolourea compounds. This theory is supported by k_{off} values determined for the Type II inhibitors 11b (RL45) and 11e (RL62) and a close Type III analogue 12a (RL29) using ac-p38α (Table 6). Direct K_{d} measurements revealed that the affinity of these compounds ranks as follows: 12a>11e>11b, which correlates predominantly with the measured off rates, or residence times for each compound. It is likely that the before mentioned water-mediated hydrogen bonding network together with the π-π interaction of Phe169 of the DFG motif with the quinazoline of 11e helps to better stabilize its Type II binding mode, thus explaining its slower off rate and lower K_{d} in comparison to 11b (K_{d}=k_{off}/kₒₙ). Compound 5 also shares a central phenyl moiety with a 1,4-para substitution pattern and dissociates from p38α at a similar rate. In contrast to cSrc, where DFG-out binders dissociate faster, Type III ligands such as 12a have longer residence times in p38α thereby explaining their higher affinity in comparison to 11b, 11e and 15. The fast dissociation of sorafenib, which also has a central phenyl moiety similar to 11b and 11e, is most likely due to the fact that it does not occupy as much of the allosteric pocket as these compounds. These higher off rates for sorafenib are nicely balanced with faster binding rates as well (data not shown), thereby maintaining a high affinity of sorafenib for p38α.

It should also be noted that 12a has a p-chlorophenyl moiety which better occupies the hydrophobic sub-pocket behind the gatekeeper residue of p38α and may slow dissociation of the ligand. Similarly, the Type II inhibitor BIRB-796 contains a naphthyl moiety at this position, resulting in slower dissociation rates than its phenyl analog 5 and the highest affinity binding to p38α.

### Example 27

### Comparison of Results Obtained with p38 and cSrc and Outlook

Inhibitor selectivity and the emergence of drug resistance remain fundamental challenges in the development of kinase inhibitors that are effective in long-term treatments. Here Applicants present a robust new method for detecting and quantifying the binding of different types of kinase inhibitors. Applicants generated a fluorescent-labeled kinase to monitor conformational changes in the activation loop, allowing for the discrimination of allosteric binders that stabilize the inactive DFG-out conformation of the tyrosine kinase cSrc. Applicants used this assay in a screening initiative and identified pyrazoloureas as weak cSrc binders. Although the binding mode of these Type III inhibitors is isostructural in cSrc and p38α (Pargellis et al., 2002) it is unclear why the affinity of these compounds for cSrc is 3 orders of magnitude poorer than for p38α. Real-time kinetic measurements of the binding of the Type II inhibitor (11a) to fluorescent-cSrc suggest that the reason could lie in conformational kinetics. The binding equilibrium between cSrc and (11a) is reached within 30 sec, whereas up to 300 sec is needed for the same compound to achieve binding equilibrium with p38α (FIG. 27). The slower on rates of Type II and III compounds is typical for p38α and has been well-documented (Pargellis et al., 2002). For Applicants' kinetic measurements, cSrc was in its phosphorylated active state whereas p38α is unphosphorylated when expressed and purified from bacteria. Furthermore, the cSrc kinase domain used in this study does not contain SH domains, leaving helix C free to more readily sample its active and inactive conformations (Levinson et al., 2006), while helix C in p38α stays in a conformation analogous to that of inactive cSrc. Therefore, cSrc is likely to more rapidly sample its conformational space but spends less total time in the inactive conformation than p38α. This means that conformations able to bind pyrazoloureas are quickly created in cSrc (resulting in the observed faster binding characteristics) but come at a higher entropic cost than in p38α (resulting in the poorer affinity to cSrc). Additionally, the conformational exchange undergone by helix C in cSrc makes E310—a residue making key hydrogen bonding interactions with the urea moiety of pyrazoloureas (FIGS. 20 and 23)—less available than its counterpart in p38α (E71), further contributing to the weak affinity of pyrazoloureas for cSrc.

Despite weak binding to cSrc in comparison to p38α, the initial pyrazolourea hits proved to be excellent starting points for the development of more potent cSrc inhibitors. Based on the analysis of structures of these Type III scaffolds in cSrc and on structures of cSrc in complex with quinazoline-based Type I inhibitors, Applicants designed quinazoline-pyrazolourea hybrid compounds which proved to be excellent cSrc inhibitors. Several derivatives were synthesized to explore SAR based on Applicants' prediction that the geometry of these compounds would govern their preferential binding to either cSrc or p38α and circumvent larger gatekeeper residues which are known to commonly cause drug resistance in certain cancer cell lines. Applicants were able to confirm these hypotheses using direct Kd determination (fluorescent cSrc and p38α), kinase activity assays and X-ray crystallography. The increased affinity of these compounds was not only due to the added 4-aminoquinazoline moiety to contact the hinge region of the kinase, but also due to the substitution pattern of the central phenyl moiety which is positioned near the gatekeeper residue. Although both para- and meta-substituted compounds inhibit cSrc in the low to mid nM range, only the 1,4-substituted hybrid overcame the drug resistance mutation in cSrc because this central phenyl moiety has the rotational freedom necessary to avoid a clash with the bulkier gatekeeper side chain without disturbing the arrangement of the rest of the drug molecule. The activity of these compounds in cSrc-relevant prostate cancer cell lines supports the structure-based rationale used in the design of these more selective and more potent hybrid compounds. Although it is not clear which kinases will develop point mutation-associated drug resistance under the regime of targeted therapies, it is evident that this is likely to become a major problem in the future as more kinase inhibitors are used to treat larger patient populations. As gleaned from the emergence of resistance to antimicrobial or antiviral agents by bacteria and viruses, the chemical inactivation of essential proteins creates selective pressures which increase the incidence of mutations and convey resistance. Cells carrying these mutations become more pronounced in rapidly dividing cell populations. To account for this challenge and to stimulate the design of future generation kinase inhibitors, excessive investigations are underway to provoke drug resistance formation by kinase inhibitors in model organisms to uncover future clinically-relevant mutant kinase alleles and employ them as predictive markers. Such knowledge will advance the concept of personalized cancer therapies by using the compounds best suited for the identified tumor cell type (Bradeen et al., 2006; von Bubnoff et al., 2005; Zunder et al., 2008). In an alternative approach, knowledge about which position(s) are likely to develop drug resistance relevant mutations in kinases will be crucial for the design of next generation drugs which can overcome them. Although kinases remain one of the largest classes of enzymes studied, strategies for overcoming drug resistance is a challenging task and solutions have fallen short. Crespo et al. (2008) showed that imatinib can be reengineered to minimize the entropic cost of binding to drug resistant D816V Abl mutant by promoting disorder in the DFG loop. Applicants' results illustrate a powerful alternative rationale to overcome drug resistance by generating Type II inhibitors that have the intrinsic ability to adapt to the binding site distortions induced by these mutations while also locking the kinase in an inactive conformation. Applicants are also confident that the assay presented here is a powerful tool that could be adapted to other kinases of interest and lead to the discovery of the scaffolds needed to design more potent and specific inhibitors whose efficacy will not be affected by resistance mutations.

TABLE 7
cross-reference index of compounds used in the present invention.

| | Alternative | | | |
|---|---|---|---|---|
| Compound | No. (in | Internal | Compound | Internal |
| No. | Figures) | Designation | No. | Designation |
| 3 | 1a | RL57 | 11f | RL49 |
| 4 | 1b | RL37 | 11g | RL78 |
| 5 | 1c | RL38 | 11h | RL70 |
| 6 | 1d | RL35 | 11i | RL59 |
| 7 | 1e | RL19 | 11j | RL60 |
| 8 | 2a | RL55 | 11k | RL47 |
| 9 | 2b | RL56 | 11l | RL48 |
| 10 | 2c | RL6 | 11m | RL24 |
| 11a | 3a | RL44 | 11n | RL51 |
| 11b | 3b | RL45 | 11o | RL63 |
| 11c | 3c | RL46 | 11p | RL77 |
| 11d | 3d | RL61 | 12a | RL29 |
| 11e | 3e | RL62 | 12b | RL18 |
| | | | 12c | RL17 |
| | | | 12d | RL15 |
| | | | 12e | RL34 |
| | | | 13 | RL33 |
| | | | 14a | RL36 |
| | | | 15 | RL39 |
| | | | 16a | RL43 |
| | | | 16b | RL42 |
| | | | 17a | RL41 |
| | | | 17b | RL79 |

### REFERENCES

Andersen, C. B., et al., Discovery of selective aminothiazole aurora kinase inhibitors. ACS Chem Biol, 2008. 3(3): p. 180-92.
Annis, D. A., Nazef, N., Chuang, C. C., Scott, M. P., and Nash, H. M. (2004) A general technique to rank protein-ligand binding affinities and determine allosteric versus direct binding site competition in compound mixtures, Journal of the American Chemical Society 126, 15495-15503.
Askari, N., Diskin, R., Avitzour, M., Capone, R., Livnah, O., and Engelberg, D. (2007) Hyperactive variants of p38alpha induce, whereas hyperactive variants of p38gamma suppress, activating protein 1-mediated transcription, The Journal of biological chemistry 282, 91-99.
Avitzour, M., Diskin, R., Raboy, B., Askari, N., Engelberg, D., and Livnah, O. (2007) Intrinsically active variants of all human p38 isoforms, The FEBS journal 274, 963-975.
Bagley, M. C. et al., Microwave-assisted synthesis of N-pyrazole ureas and the p38alpha inhibitor BIRB 796 for study into accelerated cell ageing. Org Biomol Chem 4 (22), 4158 (2006).
Blair, J. A. et al., Structure-guided development of affinity probes for tyrosine kinases using chemical genetics. Nat Chem Biol 3 (4), 229 (2007)
Blencke, S., Ullrich, A., and Daub, H., Mutation of threonine 766 in the epidermal growth factor receptor reveals a hotspot for resistance formation against selective tyrosine kinase inhibitors. The Journal of biological chemistry 278 (17), 15435 (2003).
Breitenlechner, C. B. et al., Crystal structures of active SRC kinase domain complexes. J Mol Biol 353 (2), 222 (2005).
Bradeen, H. A. et al., Comparison of imatinib mesylate, dasatinib (BMS-354825), and nilotinib (AMN107) in an N-ethyl-N-nitrosourea (ENU)-based mutagenesis screen: high efficacy of drug combinations. Blood 108 (7), 2332 (2006).
Bridges, A. J. et al., World patent 9738983, CAN 128:3695 (1997).
Brünger, A. T. et al., Crystallography & NMR system: A new software suite for macromolecular structure determination. Acta Crystallogr D Biol Crystallogr 54 (Pt 5), 905 (1998).
Calalb, M. B., Polte, T. R., and Hanks, S. K., Tyrosine phosphorylation of focal adhesion kinase at sites in the catalytic domain regulates kinase activity: a role for Src family kinases. Mol Cell Biol 15 (2), 954 (1995).
Caputo G A, London E. Cumulative effects of amino acid substitutions and hydrophobic mismatch upon the transmembrane stability and conformation of hydrophobic alpha-helices. Biochemistry. 2003 Mar. 25; 42(11):3275-85.
Crespo, A., Zhang, X., and Fernandez, A., Redesigning kinase inhibitors to enhance specificity. J Med Chem 51 (16), 4890 (2008).
Dar, A. C., Lopez, M. S., and Shokat, K. M., Small Molecule Recognition of c-Src via the Imatinib-Binding Conformation. Chem Biol 15 (10), 1015 (2008).
DeLano, W. L., The PyMOL Molecular Graphics System. http://www.pymol.org (2002).
de Lorimier, R. M., Smith, J. J., Dwyer, M. A., Looger, L. L., Sali, K. M., Paavola, C. D., Rizk, S. S., Sadigov, S., Conrad, D. W., Loew, L., and Helling a, H. W. (2002) Construction of a fluorescent biosensor family, Protein Sci 11, 2655-2675.
Domarkas, J. et al., The combi-targeting concept: synthesis of stable nitrosoureas designed to inhibit the epidermal growth factor receptor (EGFR). J Med Chem 49 (12), 3544 (2006).
Dumas, J., Hatoum-Mokdad, H., Sibley, R., Riedl, B., Scott, W. J., Monahan, M. K., Lowinger, T. B., Brennan, C., Natero, R., Turner, T., Johnson, J. S., Schoenleber, R., Bhargava, A., Wilhelm, S. M., Housley, T. J., Ranges, G. E., and Shrikhande, A. (2000) 1-Phenyl-5-pyrazolyl ureas: potent and selective p38 kinase inhibitors, Bioorganic & medicinal chemistry letters 10, 2051-2054.
Dumas, J., Sibley, R., Riedl, B., Monahan, M. K., Lee, W., Lowinger, T. B., Redman, A. M., Johnson, J. S., Kingery-Wood, J., Scott, W. J., Smith, R. A., Bobko, M., Schoenleber, R., Ranges, G. E., Housley, T. J., Bhargava, A., Wilhelm, S. M., and Shrikhande, A. (2000) Discovery of a new class of p38 kinase inhibitors, Bioorganic & medicinal chemistry letters 10, 2047-2050.
Emsley, P. and Cowtan, K., Coot: model-building tools for molecular graphics. Acta Crystallogr D Biol Crystallogr 60 (Pt 12 Pt 1), 2126 (2004).
Gauglitz, G. and Vo-Dinh, T. (2003). Handbook of spectroscopy. Wiley-VCH.
Gschwind, A., Fischer, O. M. and Ullrich, A., The discovery of receptor tyrosine kinases: targets for cancer therapy. Nat Rev Cancer 4 (5), 361 (2004).
Gill, A. L. et al., Identification of novel p38alpha MAP kinase inhibitors using fragment based lead generation. J Med Chem 48 (2), 414 (2005).
Heron, N. M. et al., SAR and inhibitor complex structure determination of a novel class of potent and specific Aurora kinase inhibitors. Bioorganic & medicinal chemistry letters 16 (5), 1320 (2006).
Hibbs, R. E., Talley, T. T., and Taylor, P. (2004) Acrylodan-conjugated cysteine side chains reveal conformational state and ligand site locations of the acetylcholine-binding protein, The Journal of biological chemistry 279, 28483-28491.
Jacobs, M. D., Caron, P. R., and Hare, B. J., Classifying protein kinase structures guides use of ligand-selectivity profiles to predict inactive conformations: structure of lck/imatinib complex. Proteins 70 (4), 1451 (2008).
Johnson, L. N. and Lewis, R. J. (2001)). Structural basis for control by phosphorylation. Chem. Rev. 101, 2209-2242; 40 Johnson, L. N., Noble, M. E. M. and Owen, D. J. (1996) Active and inactive protein kinases: structural basis for regulation. Cell 85, 149-158.
Kabsch, W., Automatic processing of rotation diffraction data from crystals of initially unknown symmetry and cell constants. J. Appl. Cryst. 26, 795 (1993).
Kroe, R. R.; Regan, J.; Proto, A.; Peet, G. W.; Roy, T.; Landro, L. D.; Fuschetto, N. G.; Pargellis, C. A.; Ingraham, R. H. J Med Chem 2003, 46, 4669-75.
Lakowicz, J. R. (1999). Principles of Fluorescence Spectroscopy. Kluwer Academic/Plenum Publishers
Laskowski, R. A., MacArthur, M. W., Moss, D. S., and Thornton, J. M., PROCHECK: a program to check the stereochemical quality of protein structures. J Appl Cryst 26, 283 (1993).
Larkin, M. A. et al., Clustal W and Clustal X version 2.0. Bioinformatics 23 (21), 2947 (2007).
Levinson, N. M., Seeliger, M. A., Cole, P. A. and Kuriyan J. (2008). Structural basis for the recognition of c-Src by its inactivator Csk. Cell 134; pp. 124-134.
Levinson, N. M. et al., A Src-like inactive conformation in the abl tyrosine kinase domain. PLoS Biol 4 (5), e144 (2006).
Liu, Y. and Gray, N. S., Rational design of inhibitors that bind to inactive kinase conformations. Nat Chem Biol 2 (7), 358 (2006).
Michalczyk, A. et al., Structural insights into how irreversible inhibitors can overcome drug resistance in EGFR. Bioorg Med Chem 16 (7), 3482 (2008).
Moss, N., Breitfelder, S., Betageri, R., Cirillo, P. F., Fadra, T., Hickey, E. R., Kirrane, T., Kroe, R. R., Madwed, J., Nelson, R. M., Pargellis, C. A., Qian, K. C., Regan, J., Swinamer, A., and Torcellini, C. (2007) New modifications to the area of pyrazole-naphthyl urea based p38 MAP kinase inhibitors that bind to the adenine/ATP site, Bioorganic & medicinal chemistry letters 17, 4242-4247.
Murshudov, G. N., Vagin, A. A., and Dodson, E. J., Refinement of macromolecular structures by the maximum-likelihood method. Acta Crystallogr D Biol Crystallogr 53 (Pt 3), 240 (1997).
Nienaber, V. L. et al., Discovering novel ligands for macromolecules using X-ray crystallographic screening. Nature biotechnology 18 (10), 1105 (2000).
Nowakowski, J. et al., Structures of the cancer-related Aurora-A, FAK, and EphA2 protein kinases from nanovolume crystallography. Structure 10 (12), 1659 (2002).
Owens, L. V. et al., Overexpression of the focal adhesion kinase (p125FAK) in invasive human tumors. Cancer Res 55 (13), 2752 (1995).
Ohren, J. F., Chen, H., Pavlovsky, A., Whitehead, C., Zhang, E., Kuffa, P., Yan, C., McConnell, P., Spessard, C., Banotai, C., Mueller, W. T., Delaney, A., Omer, C., Sebolt-Leopold, J., Dudley, D. T., Leung, I. K., Flamme, C., Warmus, J., Kaufman, M., Barrett, S., Tecle, H., and Hasemann, C. A. (2004) Structures of human MAP kinase kinase 1 (MEK1) and MEK2 describe novel noncompetitive kinase inhibition, Nature structural & molecular biology 11, 1192-1197.
Okram, B. et al., A general strategy for creating “inactive-conformation” abl inhibitors. Chem Biol 13 (7), 779 (2006)
Pargellis, C., Tong, L., Churchill, L., Cirillo, P. F., Gilmore, T., Graham, A. G., Grob, P. M., Hickey, E. R., Moss, N., Pay, S., and Regan, J. (2002) Inhibition of p38 MAP kinase by utilizing a novel allosteric binding site, Nature structural biology 9, 268-272.
W. A Pearson, Rapid and Sensitive Sequence Comparison with FASTP and FASTA, in Methods in Enzymology, ed. R. Doolittle Academic Press, San Diego) 183 (1990)63-98.
Read, R. J., Pushing the boundaries of molecular replacement with maximum likelihood. Acta Crystallogr D 57 (Pt 10), 1373 (2001).
Reich, M. et al., GenePattern 2.0. Nat Genet 38 (5), 500 (2006).
Regan, J., Breitfelder, S., Cirillo, P., Gilmore, T., Graham, A. G., Hickey, E., Klaus, B., Madwed, J., Moriak, M., Moss, N., Pargellis, C., Pay, S., Proto, A., Swinamer, A., Tong, L., and Torcellini, C. (2002) Pyrazole urea-based inhibitors of p38 MAP kinase: from lead compound to clinical candidate, Journal of medicinal chemistry 45, 2994-3008.
Regan, J., Pargellis, C. A., Cirillo, P. F., Gilmore, T., Hickey, E. R., Peet, G. W., Proto, A., Swinamer, A., and Moss, N. (2003) The kinetics of binding to p38MAP kinase by analogues of BIRB 796, Bioorganic & medicinal chemistry letters 13, 3101-3104.
Rendell, D. (1987). Fluorescence and Phosphorescence. Crown
Richieri, G. V., Ogata, R. T., and Kleinfeld, A. M. (1999) The measurement of free fatty acid concentration with the fluorescent probe ADIFAB: a practical guide for the use of the ADIFAB probe, Molecular and cellular biochemistry 192, 87-94.
Schaller, M. D., Biochemical signals and biological responses elicited by the focal adhesion kinase. Biochim Biophys Acta 1540 (1), 1 (2001).
Schuttelkopf, A. W. and van Aalten, D. M., PRODRG: a tool for high-throughput crystallography of protein-ligand complexes. Acta Crystallogr D Biol Crystallogr 60 (Pt 8), 1355 (2004).
Sharma, A. and Schulman, S. G. (1999). Introduction to Fluorescence Spectroscopy. Wiley interscience. Sullivan, J. E., Holdgate, G. A., Campbell, D., Timms, D., Gerhardt, S., Breed, J., Breeze, A. L., Bermingham, A., Pauptit, R. A., Norman, R. A., Embrey, K. J., Read, J., VanScyoc, W. S., and Ward, W. H. (2005) Prevention of MKK6-dependent activation by binding to p38alpha MAP kinase, Biochemistry 44, 16475-16490.
Seeliger, M. A. et al., c-Src binds to the cancer drug imatinib with an inactive Abl/c-Kit conformation and a distributed thermodynamic penalty. Structure 15 (3), 299 (2007).
Seeliger, M. A. et al., High yield bacterial expression of active c-Abl and c-Src tyrosine kinases. Protein Sci 14 (12), 3135 (2005).
Shewchuk, L. et al., Binding mode of the 4-anilinoquinazoline class of protein kinase inhibitor: X-ray crystallographic studies of 4-anilinoquinazolines bound to cyclindependent kinase 2 and p38 kinase. J Med Chem 43 (1), 133 (2000).
Shuker, S. B., Hajduk, P. J., Meadows, R. P., and Fesik, S. W., Discovering high-affinity ligands for proteins: SAR by NMR. Science 274 (5292), 1531 (1996).
Stamos, J., Sliwkowski, M. X., and Eigenbrot, C., Structure of the epidermal growth factor receptor kinase domain alone and in complex with a 4-anilinoquinazoline inhibitor. The Journal of biological chemistry 277 (48), 46265 (2002).
Taylor, S. S. and Radzio-Andzelm, E. (1994) Three protein kinase structures define a common motif. Structure 2, 345-355; 43.
Tsou, H. R. et al., 6-Substituted-4-(3-bromophenylamino)quinazolines as putative irreversible inhibitors of the epidermal growth factor receptor (EGFR) and human epidermal growth factor receptor (HER-2) tyrosine kinases with enhanced antitumor activity. J Med Chem 44 (17), 2719 (2001).
Vogtherr, M., Saxena, K., Hoelder, S., Grimme, S., Betz, M., Schieborr, U., Pescatore, B., Robin, M., Delarbre, L., Langer, T., Wendt, K. U., and Schwalbe, H. (2006) NMR characterization of kinase p38 dynamics in free and ligand-bound forms, Angewandte Chemie (International ed 45, 993-997.
von Bubnoff, N. et al., A cell-based screen for resistance of Bcr-Abl-positive leukemia identifies the mutation pattern for PD166326, an alternative Abl kinase inhibitor. Blood 105 (4), 1652 (2005)
Wan, P. T. et al., Mechanism of activation of the RAF-ERK signaling pathway by oncogenic mutations of B-RAF. Cell 116 (6), 855 (2004).
Yeatman, T. J., A renaissance for SRC. Nat Rev Cancer 4 (6), 470 (2004).
Warner, S. L. et al., Identification of a lead small-molecule inhibitor of the Aurora kinases using a structure-assisted, fragment-based approach. Mol Cancer Ther 5 (7), 1764 (2006).
Yem, A. W., Epps, D. E., Mathews, W. R., Guido, D. M., Richard, K. A., Staite, N. D., and Deibel, M. R., Jr. (1992) Site-specific chemical modification of interleukin-1 beta by acrylodan at cysteine 8 and lysine 103, The Journal of biological chemistry 267, 3122-3128.
Young, P. R., et al., Pyridinyl imidazole inhibitors of p38 mitogen-activated protein kinase bind in the ATP site. J Biol Chem, 1997. 272(18): p. 12116-21.
Karaman, M. W., et al., A quantitative analysis of kinase inhibitor selectivity. Nat Biotechnol, 2008. 26(1): p. 127-32.
Zunder, E. R. et al., Discovery of drug-resistant and drug-sensitizing mutations in the oncogenic PI3K isoform p110 alpha. Cancer Cell 14 (2), 180 (2008).

The invention is further described by the following numbered paragraphs:

1. A kinase labeled at an amino acid having a free thiol or amino group, wherein said amino acid is naturally present or introduced in the activation loop of said kinase, with

(a) a thiol- or amino-reactive fluorophore sensitive to polarity changes in its environment; or

(b) a thiol-reactive spin label, an isotope or an isotope-enriched thiol- or amino-reactive label

such that said fluorophore, spin label, isotope or isotope-enriched label does not inhibit the catalytic activity and does not interfere with the stability of the kinase.

2. The kinase of paragraph 1, which is a serine/threonine or tyrosine kinase.

3. The kinase of paragraph 1 or 2, which is p38α, MEK kinase, CSK, an Aurora kinase, GSK-3beta, cSrc, EGFR, Abl, DDR1, AKT, LCK or another MAPK.

4. The kinase of any one of paragraphs 1 to 3, wherein the amino acid to be labeled having a free thiol or amino group is cysteine, lysine, arginine or histidine.

5. The kinase of any one of paragraphs 1 to 4, wherein one or more solvent-exposed cysteines present outside the activation loop are deleted or replaced.

6. The kinase of any one of paragraphs 3 to 5, which is p38α and wherein a cysteine is introduced at position 172 of SEQ ID NO: 1 and preferably wherein the cysteines at position 119 and 162 of SEQ ID NO: 1 are replaced with another amino acid.

7. The kinase of any one of paragraphs 3 to 5, which is cSrc and wherein a cysteine is introduced at position 157 of SEQ ID NO: 2 and preferably wherein the cysteines at position 27, 233 and 246 of SEQ ID NO: 2 are replaced with another amino acid.

8. The kinase of any one of paragraphs 1 to 7, wherein the thiol- or amino-reactive fluorophore is a di-substituted naphthalene compound, a coumarin-based compound, a benzoxadiazole-based compound, a dapoxyl-based compound, a biocytin-based compound, a fluorescein, a sulfonated rhodamine-based compound, Atto fluorophores or Lucifer Yellow or derivatives thereof which exhibit a sensitivity to environmental changes.

9. The kinase of any one of paragraphs 1 to 7, wherein the thiol-reactive spin-label is a nitroxide radical.

10. A method of screening for kinase inhibitors comprising

(a) providing a kinase labeled at an amino acid having a free thiol or amino group according to any one of paragraphs 1 to 9

(b) contacting said fluorescently or spin-labeled or isotope-labeled kinase with a candidate inhibitor;

(c) recording the fluorescence emission signal at one or more wavelengths or a spectrum of said fluorescently labeled kinase of step (a) and step (b) upon excitation: or

(c)′ recording the electron paramagnetic resonance (EPR) or nuclear magnetic resonance (NMR) spectra of said spin-labeled or isotope-labeled kinase of step (a) and step (b); and

(d) comparing the fluorescence emission signal at one or more wavelengths or the spectra recorded in step (c) or the EPR or NMR spectra recorded in step (c)′;

wherein a difference in the fluorescence intensity at least one wavelength, preferably at the emission maximum, and/or a shift in the fluorescence emission wavelength in the spectra of said fluorescently labeled kinase obtained in step (c), or an alteration in the EPR or NMR spectra of said spin-labeled or isotope-labeled kinase obtained in step (c)′ indicates that the candidate inhibitor is a kinase inhibitor.

11. A method of determining the kinetics of ligand binding and/or of association or dissociation of a kinase inhibitor comprising

(a) contacting a fluorescently labeled kinase according to any one of paragraphs 1 to 9 with different concentrations of an inhibitor; or

(a)′ contacting a fluorescently labeled kinase according to any one of paragraphs 1 to 9 bound to an inhibitor with different concentrations of unlabeled kinase;

(b) recording the fluorescence emission signal at one or more wavelengths or a spectrum of said fluorescently labeled kinase for each concentration upon excitation;

(c) determining the rate constant for each concentration from the fluorescence emission signals at one or more wavelengths or the spectra recorded in step (b); or

(c1) determining the K_{d} from the fluorescence emission signal at one or more wavelengths or the spectra recorded in step (b) for each concentration of inhibitor; or

(c2) determining the Kₐ from the fluorescence emission signal at one or more wavelengths or the spectra recorded in step (b) for each concentration of unlabelled kinase;

(d) directly determining the kₒₙ and/or extrapolating the k_{off} from the rate constants determined in step (c) from the signals or spectra for the different concentrations of inhibitor obtained in step (b); or

(d)′ directly determining the k_{off} and/or exptrapolating the kₒₙ from the rate constants determined in step (c) from the signals or spectra for the different concentrations of unlabeled kinase obtained in step (b); and

(e) optionally calculating the K_{d} and/or Ka from kₒₙ and k_{off} obtained in step (d) or (d)′.

12. A method of determining the dissociation or association of a kinase inhibitor comprising

(a) contacting a spin-labeled or isotope-labeled kinase according to any one of paragraphs 1 to 9 with different concentrations of an inhibitor; or

(a)′ contacting a spin-labeled or isotope-labeled kinase according to any one of paragraphs 1 to 9 bound to an inhibitor with different concentrations of unlabelled kinase;

(b) recording the EPR or NMR spectrum of said spin-labeled or isotope-labeled kinase for each concentration of inhibitor and/or unlabelled kinase; and

(c) determining the K_{d} from the EPR or NMR spectra recorded in step (b) for the different concentrations of inhibitor; or

(c)′ determining the Kₐ from the EPR or NMR spectra recorded in step (b) for the different concentrations of unlabeled kinase.

13. A method of generating a mutated kinase suitable for the screening of kinase inhibitors comprising

(a) replacing solvent exposed amino acids having a free thiol or amino group, if any, present in a kinase of interest outside the activation loop or amino acids having a free thiol or amino group at an unsuitable position within the activation loop with an amino acid not having a free thiol or amino group;

(b) mutating an amino acid in the activation loop of said kinase of interest to an amino acid having a free thiol or amino group if no amino acid having a free thiol or amino group is present in the activation loop;

(c) labeling the kinase of interest with a thiol- or amino-reactive fluorophore sensitive to polarity changes in its environment, a thiol-reactive spin label, an isotope or an isotope-enriched thiol- or amino-reactive label such that said fluorophore, spin label, isotope or isotope-enriched label does not inhibit the catalytic activity of the kinase and/or does not interfere with the stability of the kinase;

(d) contacting the kinase obtained in step (c) with a known inhibitor of said kinase; and

(e) recording the fluorescence emission signal at one or more wavelengths or a spectrum of said fluorescently labeled kinase of step (c) and (d) upon excitation or

(e)′ recording the EPR or NMR spectra of said spin-labeled kinase of step (c) and (d);

(f) comparing the fluorescence emission spectra recorded in step (e) or the EPR or

NMR spectra recorded in step (e)′;

wherein a difference in the fluorescence intensity at least one wavelength, preferably at the emission maximum, and/or a shift in the fluorescence emission wavelength in the spectra of said fluorescently labeled kinase obtained in step (e), or an alteration in the EPR or NMR spectra of said spin-labeled or isotope-labeled kinase obtained in step (e)′ indicates that the kinase is suitable for the screening for kinase inhibitors.

14. The method of any one of paragraphs 10 to 13, wherein the kinase inhibitor binds either partially or fully to the allosteric site adjacent to the ATP binding site of the kinase.

15. A method for identifying a kinase inhibitor binding either partially or fully to the allosteric site adjacent to the ATP binding site of a kinase comprising

(a) screening for an inhibitor according to the method of paragraph 10, and

(b) determining the rate constant of an inhibitor identified in step (a), wherein a rate constant of <0.140 s⁻¹ determined in step (b) indicates that the kinase inhibitor identified binds either partially or fully to the allosteric site adjacent to the ATP binding site of the kinase.

16. The kinase of any one of paragraphs 1 to 9 or the method of any one of paragraphs 10 to 15, wherein the kinase is labeled at a cysteine naturally present or introduced in the activation loop.

17. The method of any one of paragraphs 10 or 13 to 16, further comprising optimizing the pharmacological properties of a compound identified as inhibitor of said kinase.

18. The method of paragraph 17, wherein the optimization comprises modifying an inhibitor identified as inhibitor of said kinase to achieve:

a) modified spectrum of activity, organ specificity, and/or

b) improved potency, and/or

c) decreased toxicity (improved therapeutic index), and/or

d) decreased side effects, and/or

e) modified onset of therapeutic action, duration of effect, and/or

f) modified pharmacokinetic parameters (absorption, distribution, metabolism and excretion), and/or

g) modified physico-chemical parameters (solubility, hygroscopicity, color, taste, odor, stability, state), and/or

h) improved general specificity, organ/tissue specificity, and/or

i) optimized application form and route by
a. esterification of carboxyl groups, or
b. esterification of hydroxyl groups with carboxylic acids, or
c. esterification of hydroxyl groups to, e.g. phosphates, pyrophosphates or sulfates or hemi-succinates, or
d. formation of pharmaceutically acceptable salts, or
e. formation of pharmaceutically acceptable complexes, or
f. synthesis of pharmacologically active polymers, or
g. introduction of hydrophilic moieties, or
h. introduction/exchange of substituents on aromates or side chains, change of substituent pattern, or
i. modification by introduction of isosteric or bioisosteric moieties, or
j. synthesis of homologous compounds, or
k. introduction of branched side chains, or
l. conversion of alkyl substituents to cyclic analogues, or
m. derivatization of hydroxyl groups to ketales, acetales, or
n. N-acetylation to amides, phenylcarbamates, or
o. synthesis of Mannich bases, imines, or
p. transformation of ketones or aldehydes to Schiff's bases, oximes, acetales, ketales, enolesters, oxazolidines, thiazolidines
   or combinations thereof.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A kinase labeled at an amino acid having a free thiol or amino group, wherein said amino acid is naturally present or introduced in the activation loop of said kinase, comprising
(a) a thiol- or amino-reactive fluorophore sensitive to polarity changes in its environment; or
(b) a thiol-reactive spin label, an isotope or an isotope-enriched thiol- or amino-reactive label such that said fluorophore, spin label, isotope or isotope-enriched label does not inhibit the catalytic activity and does not interfere with the stability of the kinase.

2. The kinase of claim 1, which is a serine/threonine or tyrosine kinase.

3. The kinase of claim 1, which is p38α, MEK kinase, CSK, an Aurora kinase, GSK-3beta, cSrc, EGFR, Abl, DDR1, AKT, LCK or another MAPK.

4. The kinase of claim 1, wherein the amino acid to be labeled having a free thiol or amino group is cysteine, lysine, arginine or histidine.

5. The kinase of claim 1, wherein one or more solvent-exposed cysteines present outside the activation loop are deleted or replaced.

6. The kinase of claim 3, which is p38α and wherein a cysteine is introduced at position 172 of SEQ ID NO: 1 and preferably wherein the cysteines at position 119 and 162 of SEQ ID NO: 1 are replaced with another amino acid.

7. The kinase of claim 3, which is cSrc and wherein a cysteine is introduced at position 157 of SEQ ID NO: 2 and preferably wherein the cysteines at position 27, 233 and 246 of SEQ ID NO: 2 are replaced with another amino acid.

8. The kinase of claim 1, wherein the thiol- or amino-reactive fluorophore is a di-substituted naphthalene compound, a coumarin-based compound, a benzoxadiazole-based compound, a dapoxyl-based compound, a biocytin-based compound, a fluorescein, a sulfonated rhodamine-based compound, Atto fluorophores or Lucifer Yellow or derivatives thereof which exhibit a sensitivity to environmental changes.

9. The kinase of claim 1, wherein the thiol-reactive spin-label is a nitroxide radical.

10. A method of screening for kinase inhibitors comprising
(a) providing a kinase labeled at an amino acid having a free thiol or amino group according to claim 1
(b) contacting said fluorescently or spin-labeled or isotope-labeled kinase with a candidate inhibitor;
(c) recording the fluorescence emission signal at one or more wavelengths or a spectrum of said fluorescently labeled kinase of step (a) and step (b) upon excitation: or
(c)′ recording the electron paramagnetic resonance (EPR) or nuclear magnetic resonance (NMR) spectra of said spin-labeled or isotope-labeled kinase of step (a) and step (b); and
(d) comparing the fluorescence emission signal at one or more wavelengths or the spectra recorded in step (c) or the EPR or NMR spectra recorded in step (c)′;
wherein a difference in the fluorescence intensity at least one wavelength, preferably at the emission maximum, and/or a shift in the fluorescence emission wavelength in the spectra of said fluorescently labeled kinase obtained in step (c), or an alteration in the EPR or NMR spectra of said spin-labeled or isotope-labeled kinase obtained in step (c)′ indicates that the candidate inhibitor is a kinase inhibitor.

11. A method of determining the kinetics of ligand binding and/or of association or dissociation of a kinase inhibitor comprising
(a) contacting a fluorescently labeled kinase according to claim 1 with different concentrations of an inhibitor; or
(a)′ contacting a fluorescently labeled kinase according to claim 1 bound to an inhibitor with different concentrations of unlabeled kinase;
(b) recording the fluorescence emission signal at one or more wavelengths or a spectrum of said fluorescently labeled kinase for each concentration upon excitation;
(c) determining the rate constant for each concentration from the fluorescence emission signals at one or more wavelengths or the spectra recorded in step (b); or
(c1) determining the K_{d} from the fluorescence emission signal at one or more wavelengths or the spectra recorded in step (b) for each concentration of inhibitor; or
(c2) determining the Kₐ from the fluorescence emission signal at one or more wavelengths or the spectra recorded in step (b) for each concentration of unlabelled kinase;
(d) directly determining the kₒₙ and/or extrapolating the k_{off} from the rate constants determined in step (c) from the signals or spectra for the different concentrations of inhibitor obtained in step (b); or
(d)′ directly determining the k_{off} and/or extrapolating the kₒₙ from the rate constants determined in step (c) from the signals or spectra for the different concentrations of unlabeled kinase obtained in step (b); and
(e) optionally calculating the K_{d} and/or Ka from kₒₙ and k_{off} obtained in step (d) or (d)′.

12. A method of determining the dissociation or association of a kinase inhibitor comprising
(a) contacting a spin-labeled or isotope-labeled kinase according to claim 1 with different concentrations of an inhibitor; or
(a)′ contacting a spin-labeled or isotope-labeled kinase according to claim 1 bound to an inhibitor with different concentrations of unlabelled kinase;
(b) recording the EPR or NMR spectrum of said spin-labeled or isotope-labeled kinase for each concentration of inhibitor and/or unlabelled kinase; and
(c) determining the K_{d} from the EPR or NMR spectra recorded in step (b) for the different concentrations of inhibitor; or
(c)′ determining the Kₐ from the EPR or NMR spectra recorded in step (b) for the different concentrations of unlabeled kinase.

13. A method of generating a mutated kinase suitable for the screening of kinase inhibitors comprising
(a) replacing solvent exposed amino acids having a free thiol or amino group, if any, present in a kinase of interest outside the activation loop or amino acids having a free thiol or amino group at an unsuitable position within the activation loop with an amino acid not having a free thiol or amino group;
(b) mutating an amino acid in the activation loop of said kinase of interest to an amino acid having a free thiol or amino group if no amino acid having a free thiol or amino group is present in the activation loop;
(c) labeling the kinase of interest with a thiol- or amino-reactive fluorophore sensitive to polarity changes in its environment, a thiol-reactive spin label, an isotope or an isotope-enriched thiol- or amino-reactive label such that said fluorophore, spin label, isotope or isotope-enriched label does not inhibit the catalytic activity of the kinase and/or does not interfere with the stability of the kinase;
(d) contacting the kinase obtained in step (c) with a known inhibitor of said kinase; and
(e) recording the fluorescence emission signal at one or more wavelengths or a spectrum of said fluorescently labeled kinase of step (c) and (d) upon excitation or
(e)′ recording the EPR or NMR spectra of said spin-labeled kinase of step (c) and (d);
(f) comparing the fluorescence emission spectra recorded in step (e) or the EPR or NMR spectra recorded in step (e)′;
wherein a difference in the fluorescence intensity at least one wavelength, preferably at the emission maximum, and/or a shift in the fluorescence emission wavelength in the spectra of said fluorescently labeled kinase obtained in step (e), or an alteration in the EPR or NMR spectra of said spin-labeled or isotope-labeled kinase obtained in step (e)′ indicates that the kinase is suitable for the screening for kinase inhibitors.

14. The method of claim 10, wherein the kinase inhibitor binds either partially or fully to the allosteric site adjacent to the ATP binding site of the kinase.

15. A method for identifying a kinase inhibitor binding either partially or fully to the allosteric site adjacent to the ATP binding site of a kinase comprising
(a) screening for an inhibitor according to the method of claim 10, and
(b) determining the rate constant of an inhibitor identified in step (a),
wherein a rate constant of <0.140 s⁻¹ determined in step (b) indicates that the kinase inhibitor identified binds either partially or fully to the allosteric site adjacent to the ATP binding site of the kinase.

16. The kinase of claim 1, wherein the kinase is labeled at a cysteine naturally present or introduced in the activation loop.

17. The method of claim 10, further comprising optimizing the pharmacological properties of a compound identified as inhibitor of said kinase.

18. The method of claim 17, wherein the optimization comprises modifying an inhibitor identified as inhibitor of said kinase to achieve:
a) modified spectrum of activity, organ specificity, and/or
b) improved potency, and/or
c) decreased toxicity (improved therapeutic index), and/or
d) decreased side effects, and/or
e) modified onset of therapeutic action, duration of effect, and/or
f) modified pharmacokinetic parameters (absorption, distribution, metabolism and excretion), and/or
g) modified physico-chemical parameters (solubility, hygroscopicity, color, taste, odor, stability, state), and/or
h) improved general specificity, organ/tissue specificity, and/or
i) optimized application form and route by
a. esterification of carboxyl groups, or
b. esterification of hydroxyl groups with carboxylic acids, or
c. esterification of hydroxyl groups to, e.g. phosphates, pyrophosphates or sulfates or hemi-succinates, or
d. formation of pharmaceutically acceptable salts, or
e. formation of pharmaceutically acceptable complexes, or
f. synthesis of pharmacologically active polymers, or
g. introduction of hydrophilic moieties, or
h. introduction/exchange of substituents on aromates or side chains, change of substituent pattern, or
i. modification by introduction of isosteric or bioisosteric moieties, or
j. synthesis of homologous compounds, or
k. introduction of branched side chains, or
l. conversion of alkyl substituents to cyclic analogues, or
m. derivatization of hydroxyl groups to ketales, acetales, or
n. N-acetylation to amides, phenylcarbamates, or
o. synthesis of Mannich bases, imines, or
p. transformation of ketones or aldehydes to Schiff's bases, oximes, acetales, ketales, enolesters, oxazolidines, thiazolidines
or combinations thereof.

19. The method of claim 10, wherein the kinase is labeled at a cysteine naturally present or introduced in the activation loop.


SEQUENCE LISTING (2 sequences)

SEQ ID NO: 1 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 2 - protein, 283 aa, Artificial sequence
  source: /note=“Description of artificial sequence chicken c-SRC kinase domain residues 251-533”
QTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGEMGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKDPEERPTFEYLQAFLEDYFTSTEPQYQPGENL